(19) United States
(12) Patent Application Publication
(10) Pub. No.: US 2022/0344001 A1
(43) Pub. Date: Oct. 27, 2022

(54) System and Method for Reduction of Technical Variability and Extraction of Biological Signal from Nucleic Acid Sequencing Data

(71) Applicant: Zeljko Dzakula, San Diego, CA (US)

(72) Inventor: Zeljko Dzakula, San Diego, CA (US)

(73) Assignee: Zeljko Jovan Dzakula, San Diego, CA (US)

(21) Appl. No.: 17/227,382

(22) Filed: Apr. 12, 2021

(51) Int. Cl. G16B 20/20; C12Q 1/6869; C12Q 1/6806

(52) U.S. Cl. CPC G16B 20/20; C12Q 1/6869; C12Q 1/6806

(57) ABSTRACT

Disclosed herein is a system and method for reducing technical noise in measured nucleic acid sequencing data, for quantifying presence and amount of contaminating nucleic acids originating from individuals other than the proband, for determining the copy number of entire chromosomes or subchromosomal sections, and for clustering single cells with similar copy number profiles, in one or a small set of cells, or from fragmented DNA, where an annotated reference genome sequence is available. In accordance with one embodiment, contaminating nucleic acids are quantified at a plurality of loci using measured allele counts. In another embodiment, the whole chromosome aneuploidy or copy number of a subchromosomal section can be determined from the measured counts of sequence reads mapped to the reference genome.

### RELATED PATENT APPLICATIONS

This application claims the benefit of U.S. Provisional Patent Application No. 63/012,256, filed Apr. 19, 2020, which is incorporated by reference herein in its entirety.

### FIELD OF THE INVENTION

The field of invention covers analysis of human DNA for the purpose of detecting disease-causing aberrations in adult and/or prenatal or neonatal individuals, as well as sample tracking, quantification of sample impurities, and measurement of fetal fraction in maternal ccfDNA.

The present invention relates to single-cell DNA analyses (scDNA) and in particular to CNV and SNP aberrations in cancerous cells. It also relates to fetal contribution to circulating cell-free DNA (ccfDNA) isolated from maternal plasma and sequenced for the purpose of non-invasive prenatal testing (NIPT).

The invention improves multiple aspects of scDNA and ccfDNA data processing, including estimation of GC bias, reduction or elimination of systematic and stochastic errors from measured whole genome resequencing data, scaling of scDNA copy-number profiles, detection of features in CNV profiles, clustering of single cells based on similarity of their CNV profiles, measurement of sample impurity levels and fetal fractions based on measured SNP allele frequencies, detection of fetal copy-number variants based on measured SNP allele frequencies in ccfDNA, haplotyping of scDNA and fetal component of ccfDNA based on phased parental SNPs and measured scDNA or ccfDNA SNP allele frequencies.

### BACKGROUND OF THE INVENTION

What is needed is a collection of accurate algorithms to successfully extract biological signal from technical noise.

In one aspect of the invention, methods make use of Fourier coefficients of the distribution of read counts to accurately quantify GC bias in a highly complex CNV profile of a cancerous cell.

In one aspect of the invention, methods reduce or eliminate technical variability from copy-number profiles. The removal of variability includes both systematic errors and stochastic noise. The resulting normalized profile enables more accurate scaling and detection of CNVs and whole-chromosome aneuploidies, both in scDNA and ccfDNA scenarios, as well as measurement of fetal fractions in ccfDNA.

In one aspect of the invention, methods accurately estimate the scale of the highly complex cancerous scDNA copy number profile.

In one aspect of the invention, methods accurately measure fetal fraction in normalized copy number profile extracted from a ccfDNA sample.

In one aspect of the invention, methods segment a CNV profile into homogeneous regions using a combination of Fourier coefficients and differential variance ratio.

In one aspect of the invention, methods enhance cell clustering by quantifying the degree of similarity among scDNA copy number profiles using a combination of Fourier expansion and differential variance ratio.

In one aspect of the invention, methods quantify the level of sample contamination based on measured SNP allele frequencies.

In one aspect of the invention, methods measure fetal fraction in ccfDNA based on measured SNP allele frequencies.

In one aspect of the invention, methods detect fetal copy number variants and whole chromosome aneuploidies in ccfDNA based on measured SNP allele frequencies.

In one aspect of the invention, methods perform haplotyping of scDNA (such as IVF quality control biopsy sample) or NIPT fetal DNA in a ccfDNA sample based on known parental phasing information and on measured SNP allele frequencies. The phasing of fetal DNA enables detection of monogenic diseases without targeting disease loci. Instead, it is enough to phase SNPs that flank the disease loci.

The things that are needed will be put forth as solutions in the next section.

### OBJECTS OF THE INVENTION

The overall object of this invention is to enhance accuracy and robustness of detection and quantification of biological features in human genomic data against the background of technical and stochastic errors introduced by measurement techniques, instrumentation, laboratory handling, environmental factors, and chance.

It is an object of this invention to achieve accurate quantification of linear and quadratic coefficients of the parabola describing GC bias in copy-number profiles of highly complex cancerous cells. Due to multiple ploidy levels in cancerous DNA, standard regression is insufficient for the task. The goal is to formulate a generalizable mathematical framework that works equally successfully with the entire spectrum of profile complexity, ranging from nearly-diploid cells to cancer cells with highly rearranged genomes.

It is also an object of this invention to reduce or eliminate systematic and stochastic errors present in whole genome resequencing data, both from scDNA and ccfDNA experiments.

Another object of this invention is to improve accuracy and robustness of CNV profile scaling, particularly in scDNA data obtained from cancerous cells that underwent complex genome rearrangements.

It is an object of this invention to enhance the accuracy of measured fetal fractions in ccfDNA.

It is an object of this invention to enhance detection of copy number variants in scDNA and ccfDNA.

It is an object of this invention to improve clustering of single cells based on similarities between their CNV profiles.

It is an object of this invention to quantify presence of contaminations in a DNA sample.

It is an object of this invention to enable affordable and accurate haplotyping of the fetal genome using scDNA or ccfDNA data and known parental haplotypes in order to detect transmission of monogenic diseases from the carrier (or affected) parent(s).

Still other objects and advantages of the invention will in part be obvious and will in part be apparent from the specification and drawings.

### SUMMARY OF THE INVENTION

In order to overcome systematic and stochastic errors when extracting biological signal from scDNA and ccfDNA measurements, the present disclosure applies mathematical modeling techniques applicable to both whole-genome resequencing data and to targeted SNP allele counts. The invention implements these statistical and algorithmic solutions in the context of a genomic analysis workflow, comprising sample collection, sample storage and transportation, DNA extraction, library preparation, DNA sequencing, alignment of sequenced DNA fragments to the human reference, counting of reads aligned to specific genomic locations, as well as computer software and hardware used to effectively process the count data in an automated fashion and to automatically create accurate reports regarding subject's disease status.

The invention accordingly comprises the several steps and the relation of one or more of such steps with respect to each of the others, and the apparatus embodying features of construction, combinations of elements and arrangement of parts that are adapted to affect such steps, all is exemplified in the following detailed disclosure, and the scope of the invention will be indicated in the claims.

To accurately quantify GC bias, the methods of the invention generate a distribution of read counts per bin, perform Fourier expansion of the distribution, generate a metric called Fourier Coefficient Ratio (FCR) by combining Fourier coefficients of different orders, and vary trial GC coefficients until achieving optimal FCR value.

To reduce or eliminate systematic and stochastic errors from whole genome resequencing scDNA or ccfDNA data, the methods of the invention generate posterior ploidy probability distribution and evaluate its measure of central tendency for each genomic bin satisfying predefined quality criteria. The resulting values are referred to as normalized profile values.

To improve accuracy and robustness of scDNA CNV profile scaling, the methods of the invention manipulate normalized profile values to generate a penalty function, and then vary trial scale values until the penalty reaches its optimum.

To enhance the accuracy of measured fetal fraction values in ccfDNA, the methods of the invention use normalized CNV profile values obtained by other methods of the invention. Still other methods of the invention generate likelihood or posterior probability distributions of observed SNP allele counts and maximize the likelihood or the posterior probability.

To enhance detection of copy number variants in scDNA or in ccfDNA data, the methods of the invention perform Fourier expansion of the CNV profile, combine the Fourier coefficients of the resulting series to generate Fourier Coefficient Ratio, generate differential profile consisting of successive differences of the original profile, evaluate variances of the original profile and the differential profile, generate Differential Profile Variance, and combine Fourier Coefficient Ratio with Differential Profile Variance to arrive at a measure of profile homogeneity. The measure of profile homogeneity is then used to segment the profile into homogeneous regions.

To improve clustering of single cells, the methods of the invention combine CNV profiles of individual cells in a pairwise fashion and generate a similarity metric from the combined profile's Fourier Coefficient Ratio and the combined profile's Differential Variance Ratio.

To quantify presence of contaminants in a DNA sample the methods of the invention use measured allele counts to construct posterior probability distribution of contamination levels and then evaluate the distribution's central tendency.

To accurately measure fetal fraction in a ccfDNA sample the methods of the invention use measured allele counts to construct posterior probability distribution of the fetal fraction and then evaluate the distribution's central tendency.

To enhance detection of fetal copy number variants in ccfDNA, the methods of the invention use measured SNP allele frequencies to generate posterior ploidy probability distributions and then identify the distribution's mode.

To enable affordable and accurate haplotyping of the fetal genome using scDNA or ccfDNA data and known parental haplotypes in order to detect transmission of monogenic diseases from the carrier (or affected) parent(s), the methods of the invention generate likelihoods of fetal transmission patterns per SNP locus, combine these likelihoods across genomic regions, and segment genomic regions into homogeneous subregions, thus identifying paternally and maternally inherited haplotypes and parental recombination sites.

### BRIEF DESCRIPTION OF THE DRAWINGS

For a more complete understanding of the invention, reference is made to the following description and accompanying drawings, in which:

FIG. 1 Data points: simulated GC bias coefficient values for 1,000 cells modeled after ICR's profile for cell line 22Rv-1. Gray straight lines delineate region allowed by the non-negativity constraint (above the V-shaped border formed by the two lines);

FIG. 2 Data points: residual GC bias coefficients after applying entropy minimization. V-shaped border has the same meaning as in FIG. 1. Dotted lines: ideal scenario (vanishing GC bias);

FIG. 3 Data points: residual GC bias coefficients after applying Fourier Coefficient Ratio. V-shaped border: same as in FIG. 1. Dotted lines: ideal scenario (vanishing GC bias);

FIG. 4 Curves: distribution of errors in estimated linear GC coefficients L using entropy (gray) or Fourier Coefficient Ratio (black). Vertical dotted line: ideal scenario (zero error);

FIG. 5 Curves: distribution of errors in estimated quadratic GC coefficients Q using entropy (gray) or Fourier Coefficient Ratio (black). Vertical dotted line: ideal scenario (zero error);

FIG. 6 Data points: errors in quadratic GC coefficients vs. errors in linear GC coefficients, as estimated using entropy (gray crosses) or Fourier Coefficient Ratio (black circles). Horizontal and vertical dotted lines: ideal scenario (vanishing errors);

FIG. 7 Gray curve: distribution of unnormalized bin counts in cell 544, cell line 22Rv-1. Dark-gray and black curves: distributions of bin counts after normalization using entropy (dark-gray) or Fourier Coefficient Ratio (dashed, black). The two normalized curves almost completely overlap. Vertical dotted lines: integer multiples of scale S, indicating ideal locations of density peaks;

FIG. 8 Data points: bin counts vs. GC content per bin in cell 544 (cell line 22Rv-1). Left inset: unnormalized. Center: entropy-based normalization. Right inset: normalization using Fourier Coefficient Ratio;

FIG. 9 Gray curves: unnormalized (top) and normalized CNV profiles (center—entropy and bottom—FCR) for cell 544, cell line 22Rv-1. Black curve: ground truth for 22Rv-1, downloaded from ICR and used as template when simulating bin counts. Ground truth is multiplied by scale S;

FIG. 10 Simulated GC bias coefficients in 52,437 cells (231 cell lines);

FIG. 11 Errors per cell vs. scale S (52,437 cells, 231 cell lines);

FIG. 12 Errors per cell vs. cell mean ploidy (52,437 cells, 231 cell lines);

FIG. 13 Distributions of error counts in posterior ploidy probability profiles for different mean cell ploidy ranges (52,437 cells, 231 cell lines);

FIG. 14 Top inset, gray curve: raw bin count profile for cell line A2780, cell 380. Bottom inset, gray curve: normalized bin counts (posterior ploidy probability mode) for the same cell. Black curves: ground truth. Scale: 98.38, GC coefficients: L=−2.63, Q−2.70. Mean ploidy: 2.00. Errors: 13;

FIG. 15 Data points: predicted vs. true scale values in 213 cell lines (45,879 cells). Straight white line: ideal agreement;

FIG. 16 Data points: predicted vs. true scale values in 18 cell lines (9,381 cells). Straight white line: ideal agreement. Number of data points on the main diagonal: 4,943. Number of outliers: 2,898;

FIG. 17 Distribution of mean ploidy values. Gray curve: 213 cell lines where all cells were correctly scaled. Black curve: 8 cell lines where scaling failed in more than 10% of cells;

FIG. 18 Gray lines: ground truth copy-number profiles of all cell lines that failed scaling (except cell line 5637);

FIG. 19 Data points: fraction of cells with incorrect scale estimates vs; mean ploidy;

FIG. 20 Left inset, gray curve: simulated ccfDNA copy number profile (raw bin counts divided by scale) containing a fetal CNV. Right inset, gray curve: per-bin mean of the posterior probability distribution of combined ploidy P=|[(1−f)M+fF]12 (cf. Eq. 22). Both insets, black lines: ground truth. The simulated profile consists of 1,100 bins. Each bin is 50 kb long and on average receives S=150 reads. The middle, elevated portion is 500 bin (25 Mb) long and contains fetal duplication with copy number F=3. The fetal fraction is set to f=0.20, leading to an elevation of 0.10 above the euploid level of 1. The affected region is flanked by two euploid segments (100 and 500 bins on the left and right, respectively). Raw bin counts (left inset) were randomly generated according to Poisson distribution with the mean set to S in the euploid segments or to S(1+f/2) in the elevated region. Mappability is uniformly set to m=1 and GC bias coefficients are both zero, leaving stochastic noise as the only source of deviations between scaled raw counts and ground truth (left inset). The right inset illustrates that evaluation of posterior probabilities greatly reduces the amount of noise, leading to a profile that almost coincides with ground truth;

FIG. 21 Top inset: distributions of simulated raw ccfDNA bin counts divided by scale (same data as in left insert, FIG. 20). Bottom inset: distributions of normalized bin counts abtained as mean values of posterior count distributions (same data as in right insert, FIG. 20). Both insets, black curves: euploid bin elevations; dark gray curves: distributions of affected bin elevations; gray vertical straight lines: ground truth;

FIG. 22 Homogeneity indicators: DVR (top inset), Entropy (middle inset), and Fourier Coefficient Ratio (FCR, bottom inset). Curves show distributions of values for diploid profiles (solid black), deletions (dotted black), and duplications (solid gray). One million diploid, deletion, and duplication profiles were simulated. Each profile is 100 bins long, with haploid scale set to 20. Both deletions and duplications are 20 bins long and placed in the middle of the profile. All three homogeneity indicators are evaluated for each profile. To facilitate comparisons among the three homogeneity indicators, standardization is applied according to the formula z=| [symbol] −μ(x)]/σ(x), where z is the standardized value of an indicator, [symbol] is the actual (unstandardized) value of the indicator, x is the set of unstandardized values of the same indicator observed in diploid profiles, μ(x) is the mean taken over diploid profiles, and σ(x) is the corresponding standard deviation. FCR evaluation truncated the Fourier series at the fifth order;

FIG. 23 Illustrative example of profile segmentation using Fourier Coefficient Ratio. Bin counts are simulated such that bins 1-40 and 61-90 are diploid, while bins 41-60 are triploid. Scale is set to 20. Raw counts are generated using Poisson distribution. No biases are included. FCR correctly identifies the triploid region;

FIG. 24 Distributions of Fourier Coefficient Ratio values for pairwise profile differences. Three groups of copy-number profiles were simulated: diploid profiles, profiles with a deletion, and profiles with a duplication. Each group consists of 100 profiles. Each profile is 100 bins long, with scale S set to 20. Deletions and aberrations are both 20 bins long and occupy bins 41-60. Gray curves: distributions of FCR values for pairwise differences of profiles from the same group. Black curves: distributions of FCR values for pairwise differences of profiles from different groups (solid line: euploid-vs-deletions, dashed line: euploid-vs-duplication, dotted line: deletion-vs-duplication). Before evaluating FCR, each profile difference is shifted by 1. Fourier truncation order is set to 5;

FIG. 25 Gray data points: estimated (y-axis) vs. true (x-axis) contamination levels for 1,000 simulated data sets. Solid white straight line: ideal agreement. A panel of 65 SNPs with high minor allele frequency was used to simulate allele counts for a set of 1,000 contaminated samples. The true contamination level ranges from 0 to 0.25. The dbSNP IDs, genomic positions in GRCh38, reference and alternate alleles, as well as expected total fractional coverage per locus are listed in Table 0.22. Predictions were made using posterior probability distributions extracted from input allele counts. Weights were set to 1/6 for scenarios where the contamination is heterozygous and 1/24 elsewhere. No drop-in alleles, drop-out alleles, GC bias, or reference bias were included in simulations. The simulations allowed for up to 4% drop-out loci. Loci with fewer than one allele count were filtered out, as well as loci with fewer than 10 total counts. The simulated total coverage uniformly ranges from 30,000 to 80,000 reads per sample. Allele counts were simulated using Poisson distribution with the mean determined by the relative contribution of sample and contaminant genotypes. Contamination estimate per sample was obtained as the mode of the combined log(P) profile, obtained by adding together log(P) curves for individual loci within that sample. Mean, median, and standard deviation of the differences between true and predicted values are 0.15%, 0.12%, and 0.39%, respectively;

FIG. 26 Gray data points: estimated (y-axis) vs. true (x-axis) fetal fraction levels for 1,000 simulated data sets. Solid white straight line: ideal agreement. A panel of 65 SNPs with high minor allele frequency was used to simulate allele counts for a set of 1,000 NIPT samples. The true fetal fraction level ranges from 0 to 0.25. The dbSNP IDs, genomic positions in GRCh38, reference and alternate alleles, as well as expected total coverage per locus are listed in Table ??. Predictions were made using posterior probability distributions extracted from input allele counts. No drop-in alleles, drop-out alleles, GC bias, or reference bias were included in simulations. The simulations allowed for 4% drop-out loci. Loci with fewer than one allele count were filtered out, as well as loci with fewer than 10 total counts. The simulated total coverage uniformly ranges from 30,000 to 80,000 reads per sample. Allele counts were simulated using Poisson distribution with the mean determined by the relative contribution of maternal and fetal genotypes. Fetal fraction estimate per sample was obtained as the mode of the combined log(P) profile, obtained by adding together log(P) curves for individual loci within that sample. Mean, median, and standard deviation of the differences between true and predicted values are 0.01%, 0.02%, and 0.42%, respectively;

FIG. 27 Data points: estimated vs. true fetal fraction values in a set of 300 simulated data sets. SNP allele frequencies in a panel comprising 65 loci were generated as described in main text. In addition to diploid genotypes, fetal fraction estimation included deletions and duplications. White straight line: ideal agreement;

FIG. 28 Data points: count of incorrect genotype assignments vs. depth fraction per locus;

FIG. 29 Schematic representation of an embodiment of the single cell CNV workflow;

FIG. 30 Schematic representation of an embodiment of the circulating cell-free DNA workflow involving whole genome resequencing to generate copy number profiles and detect whole chromosome fetal aneuploidy and/or subchromosomal fetal CNVs;

FIG. 31 Schematic representation of an embodiment of the circulating cell-free DNA workflow involving measured SNP allele frequencies; and

FIG. 32 Schematic representation of an embodiment of a system in which certain embodiments of the invention may be implemented.

### DETAILED DESCRIPTION OF THE PREFERRED EMBODIMENTS

### Definitions

### Abbreviations

1. NIPT: Non-Invasive Prenatal Testing.

2. scDNA: Single-cell DNA.

3. ccfDNA: Circulating cell-free DNA.

4. SNP: Single nucleotide polymorphism

5. CNV: Copy-number variant.

Detailed lists of additional abbreviations and notational symbols are included in relevant specification sections.

### Technical Description of the System

### A. GC Bias Estimation by Fourier Expansion

Notation

| | |
|---|---|
| 1. | g: GC content per bin. |
| 2. | [symbol] (g): GC distribution. May be evaluated over all |
| | bins covering the entire genome, or an arbitrary |
| | subset of genomic bins. Examples include, but are not |
| | limited to a single chromosome, |
| | all autosomal chromosomes, sex chromosomes, |
| | non-repetitive bins, and/or highly mappable bins. |
| 3. | g_{L}: Lower bound on GC content per bin. |
| 4. | g_{U}: Upper bound on GC content per bin. The lower and the |
| | upper bounds satisfy the inequality 0 ≤ g_{L} ≤ g ≤ g_{U} ≤ 1. |
| 5. | g₀: Reference GC content value inside the segment (closed |
| | interval) [g_{L}, g_{U}]: g_{L} ≤ g₀ ≤ g_{U}. The reference point go may |
| | match the median of the GC distribution [symbol] (g), |
| | the mean of the GC distribution, the mode of the |
| | GC distribution, or the midpoint between g_{L} and |
| | g_{U}. It may also be an arbitrary point anywhere |
| | within the segment [g_{L}, g_{U}]. In principle, values |
| | outside the segment [g_{L}, g_{U}] are also allowed. |
| 6. | Δ_{L}: Distance from the lower GC bound g_{L} to the |
| | reference GC point go: Δ_{L} = g₀ ≤ g_{L}. |
| 7. | Δ_{U}: Distance from the reference GC point g₀ to the |
| | upper GC bound g_{U}: Δ_{U} = g_{U} − g₀. |
| 8. | L: Linear GC coefficient. |
| 9. | Q: Quadratic GC coefficient. |
| 10. | m: Mappability per bin. |
| 11. | S: Scale (mean read counts per bin per ploidy unit). |
| 12. | X (g)): Observed read count for a bin with GC content g, |
| | divided by bin ploidy. |
| 13. | G(g) or G(g; L, Q, g₀): GC bias, modeled as parabola: |
| | G(g; L, Q, g₀) = 1 + L(g − g₀) + Q(g − g₀)². |
| | GC Content per Bin |

The variable g∈[0, 1] describes the fractional GC content per bin. In practice, the lower bound g_{L} and the upper bound g_{U} are tighter: g∈[g_{L},g_{U}], where 0≤g_{L}≤g_{U}≤1. For example, the values g_{L}=0.3,g_{U}=0.7 may be appropriate for human genome.

For convenience, we also introduce the reference GC value g₀. The value g₀ is arbitrarily chosen and may match the median GC content, the mean GC content, the mode of the GC content distribution [symbol] (g), the midpoint of the segment [g_{L},g_{U}], or any conveniently selected point on the GC axis within the interval [g_{L},g_{U}] or outside of it. The final results do not depend on the choice of g₀, but the numerical values of the model coefficients L and Q do (see below).

The gaps separating g₀ from the lower and upper bounds g_{L} and g_{U} are labeled Δ_{L} and Δ_{U}, respectively:

Δ_{L}=g₀−g_{L} (1)

Δ_{U}=g_{U}−g₀ (2)

GC Bias

To define GC bias, we first introduce three new objects: bin mappability m, overall scale S, and the random variable X(g). The parameter S represents the mean bin count across the entire genome, normalized with respect to bin ploidy. The parameter m represents bin-specific efficiency of read alignment, as known to those skilled in the art. The random variable X(g) is the observed read count per ploidy unit for a bin characterized by GC content g. GC bias G(g) is defined as the asymptotic value of the ratio between the observed read count per unit ploidy X(g) vs. the product of the scale S and the bin mappability m:

$$G ( g ) = {}_{S \to ∞} \frac{X ( g )}{mS} \qquad ( 3 )$$

Division by bin-specific mappability m separates GC-dependent bias from GC-independent bias. The limit in Eq. 3 eliminates stochastic effects of coverage depth. As a result, the function G(g) describes only the effects of GC content on read counts per bin across the copy-number profile.

The GC bias function G(g) measures the deviation of the observed read counts per bin per ploidy unit X(g) from the constant value mS. The GC bias G(g) represents systematic errors in measured bin counts due to variations in GC content per bin g. Ideally, in the absence of GC bias, G(g) identically equals one. In real measurements, read counts per bin may correlate with the bin GC content g, in which case GC bias G(g) deviates from one. In general, the function G(g) is unknown and may assume a complex analytical form reflecting the mechanistic causes of the bias. Here, GC bias is modeled phenomenologically by expanding the general function G(g) into a Taylor series around g₀ and truncating the expansion at the second order, yielding the following parabola with parameters L (linear coefficient) and Q (quadratic coefficient):

G(g;L,Q,g₀)=1+L(g−g₀)+Q(g−g₀)²,g∈[g_{L},g_{U}] (4)

The model coefficients L and Q are interpreted as the first and second derivatives, respectively, of the function G(g), taken at the point g₀ (with additional multiplier ½ in the case of Q):

$$L = {( \frac{dG ( g )}{dg} )}_{g = g 0} \qquad ( 5 )$$

$$Q = \frac{1}{2} {( \frac{{d}^{2} G ( g )}{{dg}^{2}} )}_{g = g 0} \qquad ( 6 )$$

Constraints on GC Bias Coefficients L and Q

To be physically plausible, the bias function G(g;L,Q,g₀) must be non-negative on the domain g∈[g_{L},g_{U}]. This non-negativity requirement imposes constraints on parameters L and Q:

$$Q \geq \frac{- 1}{{Δ}_{L}^{2}} + \frac{L}{{Δ}_{L}} , L \geq {L}_{0} \qquad ( 7 )$$

$$Q \geq \frac{- 1}{{Δ}_{U}^{2}} - \frac{L}{{Δ}_{U}} , L \leq {L}_{0} \qquad ( 8 )$$

The intersection point L₀ is found as follows:

$${L}_{0} = \frac{\frac{1}{{Δ}_{L}^{2}} - \frac{1}{{Δ}_{U}^{2}}}{\frac{1}{{Δ}_{L}} + \frac{1}{{Δ}_{U}}} \qquad ( 9 )$$

Profile Uniformity

In the absence of GC bias, the two GC coefficients L and Q vanish and G(g) reduces to one. This scenario is the most desirable because it is the most favorable to performance metrics for detection of copy-number variants (CNVs), including sensitivity, specificity, positive predictive value, negative predictive value, false positive rate, false negative rate, true positive rate, and true negative rate.

The importance of the GC bias lies in its impact on measured copy-number profile, which directly affects the performance of CNV detection. Higher GC bias (larger deviations of G(g) from one) mean less uniform profile, which translates to lower CNV detection performance (lower sensitivity, specificity, positive predictive rate, negative predictive rate, true positive rate, and true negative rate, as well as higher false positive and false negative rates).

In absence of biological variability, the variance of a copy number profile x can be modeled using the following expression:

$$𝕍ar ( x ) = {P}^{2} [ 𝕍ar ( m ) + 𝕍ar ( g ) {L}^{2} + 𝕍ar ( {g}^{2} ) {Q}^{4} + C ( m , g , L , Q ) ] + \frac{PE}{S} \qquad ( 10 )$$

In Eq. 10, P is the profile's ploidy, x is the bin count divided by the scale S, [symbol] ar(m) is the variance of the genomic region's mappability, [symbol] ar(g) is the variance of the GC content per bin, L and Q are the linear and quadratic GC bias coefficients, and the term C(m,g,L,Q) collects all pairwise covariance contributions, which are not negligible. The term E is close to 1. Deviations E−1 reflect presence of duplicate reads. The term P/S is the stochastic Poisson contribution, responsible for deterioration of profile evenness as the scale S (average read count per bin per chromosome copy) decreases.

Division by m and bin filtering based on m significantly reduce [symbol] ar(m). Two dominant contributions to unevenness originate from L² (with proportionality constant [symbol] ar(g)≈0.014 for all hg19 bins) and from Q⁴ (proportional to [symbol] ar(g²)≈0.004 in hg19). Larger absolute values of L and Q translate to increased profile unevenness. Accurate estimation of L and Q enables removal of these two major contributors to unevenness. Errors δ_{L} and δ_{Q} (differences between true and estimated coefficient values) result in residual unevenness proportional to [symbol] ar(g)δ_{L}²+ [symbol] ar(g²)δ_{Q}⁴ (plus correlation terms). To optimize profile evenness, estimation of bias coefficients must reduce the error terms δ_{L} and δ_{Q} as much as possible.

GC Normalization

Often, in real data, L≠0 and/or Q≠0. The resulting measured read counts per bin are then biased by the per-bin GC content g. To recover the CNV performance lost due to GC bias, one first needs to estimate model coefficients L and Q from the data. Next, the bias G(g) is computationally reduced or removed. One possible approach divides the observed counts with the estimated parabola G(g;L,Q,g₀). An alternative, novel approach is discussed below (Section B. scDNA Profile Normalization by Posterior Ploidy Probability). The computational reduction or elimination of GC bias from measured read counts is known as GC normalization.

For successful GC normalization, it is essential to accurately estimate the values of the bias coefficients L and Q from the data. This estimation is the primary focus of the currently discussed aspect of the invention (GC Bias estimation by Fourier Expansion).

Prior Art: GC Bias Coefficient Estimation in Mostly Diploid Copy-Number Profiles

When the copy-number profile is mostly diploid, as is the case in non-invasive prenatal tests (NIPT), L and Q are estimated straightforwardly. The scatter plot of X(g) vs. g in diploid profiles consists of a single ribbon of data points, representing the trend G(g) with added noise. The minor complication with under-represented sex chromosomes in male individuals is easily handled, for example by focusing only on autosomes. When the curvature of the function G(g) is negligible, L can be obtained as the slope of the linear regression of X(g) vs. g. This approach was first proposed by Dr. Lin Tang (unpublished results; private communication) and subsequently used by Željko Džakula in his invention PERUN. PERUN was essential for the successful launch of the high-throughput version of the NIPT MaterniT21, used to process at least 100,000 samples in the first year after the launch. PERUN enabled increased accuracy of fetal trisomies T21, T18, and T13 detection, earliest breakthroughs in detection of fetal sex chromosome abnormalities, fetal subchromosomal abnormalities, measurements of fetal fraction in male fetuses and in trisomy/monosomy pregnancies, as well as detection of fetal autosomal aneuploidies other than T21, T13, and T18. PERUN also enabled usage of maternal CNVs to track the sample's chain of custody, removal of maternal CNVs from NIPT profiles to reduce fetal false positives and false negatives, as well as earliest examples of maternal cancer detection in NIPT samples (Željko Džakula, unpublished results).

Quadratic: Željko Džakula's extension of PERUN.

Difficulties with GC Bias Coefficient Estimation in Non-Diploid Copy-Number Profiles

Rather than a single “ribbon” of data points, as in diploid profiles, the scatter plots of X(g) vs. g in cancer samples display multiple parallel “ribbons” of data points, one for each ploidy level. Each “ribbon” follows the same rule given by the function G(g) (plus added noise). However, unlike in diploid profiles, the observations are no longer single-valued. On the contrary, observed read counts X(g) in cancer profiles follow a multi-valued function of g, reflecting multiple ploidy levels across the genome. Consequently, the parameters L and G in cancer samples become difficult or impossible to estimate using simple linear or polynomial regression.

Prior Art: Estimation of GC Bias Coefficients by Minimizing Entropy

Currently, the only existing procedure that successfully estimates L and Q in copy-number profiles with multiple ploidy levels, such as cancer, is the minimization of entropy invented by Željko Džakula in October, 2017 for the purpose of normalizing GC bias in single-cell CNV profiles. The entropy-based procedure is routinely used to process thousands of scDNA samples per run. The current invention disclosure refers to this entropy-based prior art for comparison and to asses relative performance of the newly proposed alternative approach (Fourier-based, see below).

Željko Džakula's entropy-based procedure proceeds as follows. First, a histogram of raw read counts is created, including all bins across the genome (except those filtered out due to low mappability or other filtering criteria). An upper bound is then imposed on bin counts and the distribution's tail beyond this bound is truncated. In some embodiments, the upper bound is obtained as a product of a constant factor and the mode of the distribution of bin counts. The same upper bound is enforced in all subsequent iterations to prevent variation of the edges of the support as the optimized paraters L and Q are being varied. The resulting frequencies of counts per bin are then converted to normalized probabilities p. If GC bias is present, the density peaks in this histogram will be wider than in the absence of the GC bias. In addition, GC bias induces overlaps among peaks originating from different ploidy levels. As a result, the entropy of a GC-biased histogram of bin counts is high. The estimation of L and G therefore iteratively varies coefficient values, applies the GC correction G(g;L,Q,g₀) using trial coefficient values L and Q, and regenerates the histogram until the histogram's entropy H=−Σp log(p) is minimized. The values L and Q corresponding to the lowest found value of H are reported as GC coefficient estimates.

The previous paragraph described Željko Džakula's entropy based procedure as he originally formulated and implemented it in 2017, and as it is currently being practiced at 10X Genomics. The current invention disclosure also adds the following improvement to this prior art: Before generating the histogram of read counts, the counts are divided by bin mappability values. The resulting bin counts, modulated by mappability, are then used to generate bin count histograms and to minimize entropy. Modulation by mappability significantly improves the convergence of the entropy minimization procedure and reduces the uncertainties in final GC coefficient estimates.

Alternative Approach: Estimation of GC Bias Coefficients by Maximizing Fourier Coefficient Ratio

The current invention disclosure proposes an alternative procedure, based on Fourier expansion of the distribution of read counts per bin. The basic idea is as follows: absence of GC bias is associated with sharp probability peaks in the histogram of bin counts. The corresponding Fourier series of the histogram therefore features increased absolute values of Fourier coefficients of higher orders, relative to Fourier coefficient values of the first order. A histogram of GC-biased bin counts, on the contrary, has diffuse and/or overlapping probability peaks, corresponding to decreased higher-order Fourier coefficients relative to the first-order Fourier coefficients.

Based on the principles laid out above, the estimation of L and G proceeds as follows. First, a histogram of raw bin counts is generated. A pair of cutoff percentile values is selected. In some embodiments, the percentile cutoffs are chosen at 5% and 95%. In some embodiments, only the upper bound is enforced. Cutoff percentile values are applied to the distribution of raw bin counts to yield lower and upper bin count cutoffs i_{L} and i_{U}, respectively. In some embodiments, i_{L} is set to 1. Bin counts beyond these cutoff values are truncated. The bin count cutoffs are maintained through all subsequent iterations to prevent variation of the edges of the support as the optimized parameters L and Q are being varied. Next, frequencies of observed bin counts are normalized to 1, yielding normalized bin count probabilities pᵢ, i∈[i_{L},i_{U}].

Next, the closed interval of integer bin counts [i_{L},i_{U}] is transformed to generate an equivalent discrete set of equidistant real values belonging to the segment [δ,2π] such that i maps to xᵢ=iδ, where δ is the increment separating neighboring points

$${x}_{i} \text{:} δ = {x}_{i + 1} - {x}_{i} = \frac{2 π}{{i}_{U} - {i}_{L} + 1} .$$

The next step selects the Fourier truncation order N. In some embodiments, N takes the value 2, 3, . . . , or 10. In some embodiments, N takes an integer value larger than 10.

The iterative optimization is initialized by assigning zero or a conveniently chosen negative real value to the “best” penalty. In some embodiments, the “best” penalty is initialized with negative infinity.

The iterative procedure assigns trial values to GC coefficients L and Q such that the non-negativity constraints (Eqs. 7-8) are satisfied. In some embodiments, trial values (L,Q) are selected from a predefined two dimensional grid satisfying the non-negativity constraints. The trend parabola G(g;L,Q,g₀) is evaluated for the current pair of trial values (L,Q). Raw read counts are divided by bin mappability m and by the trial trend G(g;L,Q,g₀). The resulting “corrected” read counts are trimmed within bounds [i_{L},i_{U}] and used to generate normalized probabilities pᵢ of bin counts as described above.

In some embodiments, division of raw read counts by mappability may be omitted.

Next, cosine and sine Fourier coefficients are evaluated. In some embodiments, Fast Fourier Transform (FFT) is used. In some embodiments, numerical integration is performed using Darboux sums:

$$[ \begin{matrix}{a}_{n} \\ {b}_{n}\end{matrix} ] = \frac{δ}{π} {\sum }_{i = {i}_{L}}^{{i}_{U}} {P}_{i} [ \begin{matrix}cos ( {nx}_{i} ) \\ sin ( {nx}_{i} )\end{matrix} ] , n ∈ 1 , … , N \qquad ( 11 )$$

In some embodiments, the integration in Eq. 11 is performed using numerical integration methods, such as polynomial interpolation, rectangle rule, trapezoidal rule, composite rule, extended rule, iterated rule, Newton-Cotes rule, Simpson's rule, Clenshaw-Curtis quadrature, Gauss-Kronrod quadrature, generalized mid-point rule, Monte-Carlo integration, or any other quadrature formula known to those skilled in the art.

Finally, the Fourier Coefficient Ratio F₁ value corresponding to current trial grid point (L,Q) is evaluated as follows:

$${F}_{1} ( N ) = \frac{1}{{a}_{1}^{2} + {b}_{1}^{2}} {\sum }_{n = 2}^{N} ( {a}_{n}^{2} + {b}_{n}^{2} ) \qquad ( 12 )$$

In some embodiments, absolute values |aₙ| and |bₙ| are used instead of squares. In some embodiments, positive even powers other than two (4, 6, 8, . . . ) are used.

If the current value of the Fourier Coefficient Ratio F₁ exceeds the “best” penalty, both best penalty and best (L,Q) pair of values are updated.

Optimization procedure described above continues until all grid points (L,Q) are exhausted. The final best grid point (L,Q) represents the optimal solution given the data and the resolution of the (L,Q) grid.

In some embodiments, grid search is followed by additional numerical optimization to fine-tune the solution. In some embodiments, the optimal grid point is used as the initial guess for the numerical optimization. In some embodiments, the additional numerical optimization is achieved using Nelder-Mead, Gradient Descent, BFGS, L-BFGS-B, SANN, or any other optimizer known to those skilled in the art. In some embodiments, grid search is bypassed and numerical optimization starts from an arbitrarily selected initial guess. In some embodiments, the initial guess is the origin.

B. scDNA Profile Normalization by Posterior Ploidy Probability

Notation

| | |
|---|---|
| 1. | P: Bin ploidy. |
| 2. | S: Scale: average bin count per chromosome copy. |
| 3. | m: Bin mappability. |
| 4. | g: Bin GC content. |
| 5. | g₀: Reference GC content per bin. Example: g₀ = 0.45. |
| 6. | L: Linear GC bias coefficient. |
| 7. | Q: Quadratic GC bias coefficient. |
| 8. | G(g; L, Q, g₀): GC bias, modeled as parabola: G(g; L, Q, |
| | g₀) = 1 + L(g − g₀) + Q(g − g₀)². |
| 9. | n: Observed bin count. |
| 10. | [symbol] (n): Prior probability distribution for bin counts. |
| 11. | [symbol] (PS): Prior probability distribution for the product of |
| | bin ploidy P and overall scale S. |
| 12. | [symbol] (n\|P, S, m, g, L, Q): Conditional probability to observe |
| | bin count n, given the bin ploidy P, overall scale S, |
| | bin mappability m, bin GC content g, and profile- |
| | specific linear and quadratic GC bias coefficients L and Q, |
| | respectively. Also referred to as likelihood. |
| 13. | [symbol] (PS\|n, m, g, L, Q): Conditional probability for the |
| | product of ploidy P and overall scale S, given the observed |
| | bin counts n, bin mappability m, bin GC content g, and |
| | profile-specific linear and quadratic GC bias coefficients |
| | L and Q, respectively. |
| 14. | [symbol] (n, λ): Poisson distribution with parameter λ |
| | representing the mean and n the random variable. |
| 15. | [symbol] (k, k_{L}, k_{U}): Uniform distribution for integer or continuous |
| | random variable k ranging from k_{L}, to k_{U}, inclusive. |

Normalization of Single Cell and Pure Sample Copy Number Profiles

The objective of deriving a copy-number profile from whole genome resequencing data is to extract location-specific ploidy information from observed bin counts. In raw data, ploidy information is obscured by multiple potential sources of error, including overall scale estimate, stochastic noise, and systematic biases. Scale and stochastic noise are related to overall coverage depth and are treated in separate sections of the current invention disclosure. Normalization reduces or removes biases from the bin count profile, including both mappability-induced bias and GC-related bias.

Conventionally, bias removal is achieved either by applying an additive, multiplicative, or hybrid (both additive and multiplicative) correction to observed bin counts (for example, cf. PERUN). Multiplicative corrections are often suboptimal due to their tendency to inflate errors in regions where low counts are expected. By treating the GC component of the bias as additive, rather than multiplicative, hybrid approaches (such as PERUN) greatly improve uniformity of normalized profiles. However, the mappability-related portion of the bias still has multiplicative character, thus suffering from error inflation. The current invention disclosure proposes an alternative approach, based on posterior bin count probabilities. The new approach naturally accounts for stochastic nature of observed bin counts and produces normalized profiles that are significantly less prone to error inflation than existing multiplicative or hybrid approaches.

When removing systematic errors related to mappability and GC content, the scale S may but does not have to be available. If no scale estimate exists at the time when GC/mappability normalization is performed, the resulting normalized profile represents the product of overall scale S and bin-specific ploidy P. When a scale estimate exists, S can be trivially factored out of the normalized product PS, directly yielding ploidy profile P. We first describe the former case, where normalization generates the product PS of the unknown ploidy P and unknown S.

The observed read count n in a bin characterized by mappability m and GC content g is a random variable that follows Poisson distribution [symbol] (n|P,S,m,g,L,Q), where L and Q represent the profile-specific linear and quadratic GC bias coefficients. The expectation of this conditional probability equals λ=PSmG(g;L,Q,g₀), where G(g;L,Q,g₀)=1+L(g−g₀)+Q(g−g₀)² is the parabola describing the profile-specific GC bias and g₀ is an arbitrarily chosen reference point on the GC content axis.

The prior distribution for the product PS can be modeled as continuous uniform distribution [symbol] (PS,0,Nₘₐₓ), where Nₘₐₓ is the maximum observed count. This simple model applies to the first bin in each chromosome.

For bins other than the first bin in a chromosome, the prior for the product PS is constructed as a mixture of the uniform prior and the posterior previously obtained for the preceding bin. The mixture coefficient allows balancing robustness with respect to noise vs. sensitivity to ploidy changes.

The prior distribution [symbol] (n) for the bin counts n can be modeled in several ways, including: [symbol]
1. Discrete uniform distribution [symbol] (n,0,Nₘₐₓ)
2. Poisson distribution with expectation derived from observed raw bin counts in one of the following two ways:
   a) Averaging across the entire genome
   b) Averaging within a subset of bins, such as:
      i. The chromosome on which the current bin is located
      ii. All autosomal chromosomes
      iii. All bins with the same mappability and/or GC content as the current bin
      In theory, the averaging should exclude the current bin to avoid biasing the prior. In practice, given a sufficiently large number of bins, averaging can be done once for all bins without unduly emphasizing contributions from any particular individual bin.
3. Observed distribution of raw bin counts, derived by generating a bin count histogram from:
   a) Bin counts across the entire genome
   b) Bin counts from a subset of genomic bins, such as:
      i. The chromosome on which the current bin is located
      ii. All autosomal chromosomes
      iii. All bins with the same mappability and/or GC content as the current bin
   Ideally, histogram generation excludes the current bin. In practice, histograms can be created once and reused for all bins, since a single bin in a large set of bins is unlikely to significantly affect the shape of the distribution.
4. Convolution of the distribution [symbol] (PS) for the product PS and Poisson distribution for counts centered at PSmG(g,L,Q,g₀). When evaluating the convolution integral, [symbol] (PS) can be modeled as uniform distribution [symbol] (PS,0,Nₘₐₓ).

Once the form of the prior probability [symbol] (n) for bin counts is selected, the desired posterior distribution [symbol] (PS|n,m,g,L,Q) for the product PS, given the observed bin count n, is obtained using Bayes theorem:

$$ℙ ( PS \vert n , m , g , L , Q ) = \frac{ℙ ( PS )}{ℙ ( n )} 𝒫 ( n , PSmG ( g , L , Q , {g}_{0} ) ) \qquad ( 13 )$$

In the simplest case, when both the priors for the bin count and for the product PS are modeled as uniform distributions (as in the first bin per chromosome), Eq. 13 reduces to the likelihood:

$$ℙ ( PS \vert n , m , g , L , Q ) = \frac{U ( PS , 0 , {N}_{max} )}{U ( n , 0 , {N}_{max} )} 𝒫 ( n , PSmG ( g , L , Q , {g}_{0} ) ) \qquad ( 14 )$$

$$= 𝒫 ( n , PSmG ( g , L , Q ) ) \qquad ( 15 )$$

The estimate for the product PS can then be obtained as 1) the location of the mode of the posterior distribution [symbol] (PS|n,m,g,L,Q), 2) as its mean, or 3) as its median. Along with the estimate for PS one can also obtain a measure of uncertainty in the estimate as the standard deviation, MAD (median absolute deviation), half-width, second moment, or related spread metric of the posterior [symbol] (PS|n,m,g,L,Q).

Once the posterior distribution for a given bin is constructed and used to estimate the product PS, it is also passed to the procedure that constructs the prior for PS in the next bin. As mentioned above, the prior for the next bin is a mixture of the current posterior and the uniform distribution. Mixture coefficients reflect the frequency of CNV breakpoints.

The estimate for PS obtained as described in the previous paragraph is to a large degree free of systematic biases due to mappability m and GC content g. It is also much less affected by stochastic noise than the conventional normalized profiles. As such, a collection of PS estimates obtained for a large number of bins across the genome can be used to reliably estimate the scale S, as described in a separate section below.

If a separate procedure is used to obtain an estimate for the scale S prior to normalization, the procedure described above can still be used in almost identical form to obtain final normalized and scaled ploidy profile P, rather than the product PS. One possible advantage of such approach is that the known value of the continuous variable S becomes a part of the condition in the expression for the posterior, leaving only the discrete random variable P. The prior on the ploidy P can then be more restrictive, only allowing integer P values.

C. scDNA CNV Profile Scaling

Notation

| | |
|---|---|
| 1. | S: Scale, number of reads per bin per chromosome copy. |
| 2. | P: Ploidy at a given bin. |
| 3. | PS: Product of scale and ploidy. |
| 4. | Nᵢ: The value of the product PS at genomic location (bin) i. |
| 5. | s: Trial value for scale S. |
| 6. | └x┐: Integer closest to real value x. |
| 7. | T(s): Penalty function. |
| 8. | Cᵢ: Collapsed profile value at bin i. |
| 9. | C_{L}, C_{U}: Lower and upper bound for trimming the |
| | collapsed profile Cᵢ. |
| 10. | K: Number of highest collapsed profile points C, |
| | in the neighborhood of 0.5. |
| 11. | m: Median value of K highest Cᵢ values in the |
| | neighborhood of 0.5. |
| 12. | μ: Median C, value in the flanks, representing |
| | representative elevation of the collapsed |
| | profile after baseline removal. |
| 13. | σ: MAD of the collapsed profile C, in the flanks. |
| 14. | z: Standardized value of the collapsed profile peak |
| | centered at 0.5. |
| 15. | z₀: Cutoff z value, used to decide whether to divide scale |
| | estimate by two. |

The removal of mappability- and GC-related biases from raw bin counts yields normalized PS profile, where P is the per-bin ploidy, and S is the overall scale, as described above. The ultimate objective is the ploidy profile P, rather than the product PS. The procedure described below estimates the scale S starting from the normalized PS profile as its input, thus enabling evaluation of the desired ploidy P by dividing the PS profile with the estimate S.

Ideally, the normalized profile PS consists of highly uniform segments. Since ploidy P can only take integer values, the elevations of PS segments are integer multiples of the scale S. At high enough coverage depth per bin, fluctuations around these normalized discrete segment elevations are minimal. The scale estimator takes advantage of the fact that division of the normalized profile PS with the true scale value S can only yield integer values (or values very close to integers). The estimator thus constructs a penalty (or target) function T(s) as such:

$${T ( s ) = {\sum }_{i = 1}^{M} ( \frac{{N}_{i}}{s} - ⌊ \frac{{N}_{i}}{s} ⌉ )}^{2} \qquad ( 16 )$$

The variable s in Eq. 16 represents a positive real trial value for the scale S. The sum runs over all bin indices i from 1 to the total number of bins M. The symbol Nᵢ denotes the normalized product PS at bin i. The symbol └x┐ denotes rounding of x to nearest integer.

The penalty T(s) is evaluated for all candidate scale values s from some predetermined value sₘᵢₙ to sₘₐₓ. In some embodiments, the starting value sₘᵢₙ is set to 2. In some embodiments, the upper bound sₘₐₓ is set to max(N_{Z}). In some embodiments, s is incremented from sₘᵢₙ to sₘₐₓ in integer steps. In some embodiments, s is incremented in fractional steps. In some embodiments, the range of trial values s is scanned in equal increments. In some embodiments, the range of trial values s is scanned in unequal steps. In some embodiments, any standard minimizer known to those skilled in the art is used to minimize T(s). Examples include, but are not limited to, exhausting search, stochastic search, bracketing, region elimination, halving, golden section search, and Newton-Raphson. Regardless of which minimizer is used, the scale S is assigned the trial value s that yields the lowest penalty T(s):

$$S = {arg min}_{s ∈ [ {s}_{min} , {s}_{max} ]} T ( s ) \qquad ( 17 )$$

The same estimate S can be obtained if the squared difference

$${( \frac{{N}_{i}}{s} - ⌊ \frac{{N}_{i}}{s} ⌉ )}^{2}$$

in Eq. 16 is replaced by any of its monotonic functions. For example, the penalty may be reformulated to use absolute values, rather than the square:

$$T ( s ) = {\sum }_{i = 1}^{N} \vert \frac{{N}_{i}}{s} - ⌊ \frac{{N}_{i}}{s} ⌉ \vert \qquad ( 18 )$$

Alternatively, any positive even power of the difference

$$\frac{{N}_{i}}{s} - ⌊ \frac{{N}_{i}}{s} ⌉$$

or any positive power of its absolute value can yield the same estimate S.

The procedure described above is highly accurate when the profile contains sufficiently long segments with both even and odd ploidies P. In profiles strongly dominated by even ploidies, the penalty contributions from odd segments may be drowned in noise, resulting in scale estimates that are (almost exactly) twice the true value. This can happen, for example, in a profile of a female subject with very few and very short heterozygous deletions or duplications. To overcome this difficulty, the scale estimate obtained from Eq. 17 is further tested for presence of short odd segments, as follows:

First, the normalized PS profile is divided with the scale estimate S previously obtained using Eq. 17. The resulting ratio is then shifted by its floor, to yield the collapsed profile:

$${C}_{i} = \frac{{N}_{i}}{S} - ⌊ \frac{{N}_{i}}{S} ⌋ \qquad ( 19 )$$

The collapsed profile Cᵢ takes values that range from 0 to 1:

Cᵢ∈[0,1] (20)

When the scale estimate S is close to the true scale value, the collapsed profile values gather close to zero or one, leaving the middle portion of the segment [0, 1] almost completely empty. If, however, the scale estimate S is twice the true value, the segments with odd ploidy give rise to a set of bins with Cᵢ values close to 0.5. At the same time, even-valued ploidies yield collapsed ratios close to zero or one. The presence of non-negligible number of bins with Cᵢ≈0.5 indicates that the estimate S should be divided by two to yield a value close to the true scale. The observed collapsed bin elevations are therefore used to generate a histogram, with Cᵢ values on the x-axis and their frequencies on the y-axis. In some embodiments, the histogram contains ≈100 data points, with resolution of ≈10⁻². In some embodiments, the histogram resolution scales with the number of bins.

If contributions from odd-valued ploidies are small relative to even-valued ploidies, the peak at Cᵢ≈0.5 may be overshadowed by the spikes at 0 and 1. To facilitate detection of the odd-valued peak at 0.5 (if the peak exists), even-valued peaks at 0 and 1 are removed as follows. Once the histogram of collapsed elevations Cᵢ is available, its x-axis is truncated on both ends to remove the spikes at zero and one. In some embodiments, histogram bins below C_{L} or above C_{U} are trimmed, leaving only histogram bins within the segment Cᵢ∈[C_{L},C_{U}]. In some embodiments, C_{L}=0.2 and C_{U}=0.8. In some embodiments, C_{L} and C_{U} are user-defined. In some embodiments, C_{L} and C_{U} are automatically determined based on the widths of histogram spikes at 0 and 1. The removal of spikes at 0 and 1 yields truncated histogram of collapsed elevations.

Next, the baseline of the truncated histogram is estimated and subtracted. Baseline points are obtained by selecting all histogram values corresponding to flanks of the truncated Cᵢ domain. In some embodiments, baseline points are selected within the composite, disjoint segment [0.2, 0.4]∪[0.6, 0.8]. In some embodiments, histogram points with zero frequency values are omitted from estimation of the baseline. The selected baseline points are then used as inputs to polynomial fitting procedure yielding a smooth baseline estimate. In some embodiments, the polynomial least squares procedure uses a parabola to fit the baseline. In some embodiments, a fourth-order polynomial is used to fit the baseline. The resulting polynomial baseline estimate is then subtracted from all histogram elevations to yield baseline-corrected histogram of collapsed elevations.

Next, the procedure determines whether the histogram contains a spike at Cᵢ≈0.5. First, the median μ and MAD (median absolute deviation) a of the baseline-corrected histogram frequencies in predetermined flanks of the Cᵢ domain are obtained. In some embodiments, the flanks comprise the composite, disjoint segment [0.2, 0.4]∪[0.6, 0.8]. Next, the highest K histogram points in the central region of the Cᵢ domain are identified. In some embodiments, the central region matches the segment [0.4, 0.6]. In some embodiments, the number K of selected highest histogram points is 5. In some embodiments, K=3. The median frequency m of the selected K highest central histogram frequencies is then converted to a Z value as follows:

z=(m−μ)/σ (21)

m in Eq. 21 is the median of K highest central histogram frequencies, μ is the median of histogram frequencies in the flanks, and σ is the MAD of the histogram frequencies in the flanks.

Next, the procedure uses a preset cutoff value z₀ to determine whether there is a spike at 0.5. In some embodiments, z₀=6. In some embodiments, z₀=3. If z<z₀, there is no evidence supporting existence of a spike at Cᵢ≈0.5, and null hypothesis (S is correct) is not rejected. If z≥z₀, null hypothesis is rejected, meaning that the histogram indeed contains a spike at 0.5 and the current scale estimate S is twice the true value. If the collapsed elevation data suggest that this alternative hypothesis should be accepted, the scale estimate is corrected by division with two: S←S/2.

The above procedure yields correct scale estimates in most profiles. The only exceptions are profiles consisting entirely of even-valued ploidies. Examples include replicating cells at the end of S phase, neuronal cells in G2 phase, or cells that underwent unscheduled endoreplication, as well as some ATCC cell lines, such as MOLT-16, MOLT-13, IM-9, LOUCY, GP5D, FARAGE, DAUDI, and ARPE-19. Similar situation results when two or more cells with identical karyotypes are captured in the same cell bead or share the same cell barcode, making their sequencing data indistinguishable. In most such cases, copy number alone is insufficient to determine correct scale and the scale estimate obtained as described above is unavoidably an integer multiple of the true scale (usually double). However, in the special case of an ideal female profile, an additional assumption that the subject's genome is diploid can remove the degeneracy and allow for correct scale estimation. In an ideal female profile with no deletions or duplications, all autosomal bins have ploidy two, as do the X chromosomal bins. The absence of chr Y in such profiles translates to zero ploidy values for chr Y bins (ignoring the interference from chr Y bins that are similar to chr X regions). Under these circumstances, the PS profile only takes two values: zero (within chr Y) or some positive constant real number (elsewhere), plus noise. Similar situation exists if homozygous deletions are present in autosomal or chr X regions, but heterozygous features are absent. This scenario also includes diploid-haploid hydatiform moles, such as HM1 or HM13. To allow for correct scale estimation in such cases, the procedure is as follows.

If z<z₀, an additional test is performed to answer the question whether the normalized PS profile assumes one or multiple distinct positive values (in addition to zero, and disregarding noise). If there are multiple distinct positive values, no further actions are taken, and the value of S obtained by minimizing the penalty T(s) is used to extract ploidy from the PS profile.

If, on the other hand, z<z₀ and PS only takes a single positive value (in addition to zero), the additional assumption that the genome is diploid implies that S should be divided by 2 to yield the best estimate for the scale: S←S/2. This step is correct only if the diploid assumption is true, and will lead to an incorrect result in a haploid genome (such as gamete). To avoid reporting erroneous scale values in haploid cells, the procedure allows a user-defined flag indicating whether the input biological material is haploid (as in sperm or egg cells). If the haploid flag is set to true, the last test (single or multiple PS levels) is not performed.

D. ccfDNA Profile Normalization by Posterior Ploidy Probability

Notation and Abbreviations

| | |
|---|---|
| 1. | NIPT: Non-Invasive Prenatal Test. |
| 2. | ccfDNA: Circulating cell-free DNA. |
| 3. | S: Scale. Normalizes diploid genomic regions to one. |
| 4. | M: Maternal ploidy at a given bin. |
| 5. | F: Fetal ploidy. |
| 6. | f: Fetal fraction. |
| 7. | g: Fractional GC content per bin. |
| 8. | g₀: Representative, arbitrarily chosen mid-point inside the GC content range. |
| 9. | L: Linear GC bias coefficient. |
| 10. | Q: Quadratic GC bias coefficient. |
| 11. | G(g, L, Q, g₀): Parabola describing GC bias. |
| 12. | W = (1 − f)M + f F: Combined maternal and fetal bin ploidies. |
| 13. | [symbol] (n\|S, M, F, f, g, m, L, Q): Likelihood (conditional probability of observing n |
| | reads, given the scale S, maternal ploidy M, fetal ploidy F, fetal fraction f, |
| | mappability m, linear GC coefficient L, and quadratic GC coefficient Q). The |
| | shortened notation [symbol] (n\|S, W, g, m, L, Q) has the same meaning. |
| 14. | [symbol] (n; λ): Poisson distribution of the non-negative integer random variable n |
| | with the mean λ. |
| 15. | [symbol] (W\|n): Posterior distribution of combined ploidy values W, given the observed |
| | raw counts n. This is shorthand notation for [symbol] (W\|n, S, g, m, L, Q). |

In NIPT ccfDNA samples, the dominant maternal contribution to bin count profile is usually diploid, with minor deviations due to maternal and/or fetal CNVs. The overwhelmingly diploid character of the profile makes the estimation of the scale trivial: S is obtained as the mean or the median of the autosomal portion of the maternal genome.

Unlike in scDNA analyses of cancer CNV profiles, where the scale usually represents the number of reads per bin per chromosome copy, the scale in the ccfDNA NIPT case often represents read count per bin per pair of chromosomes. Typically, scaling is performed by dividing all raw bin counts with the mean or median of the autosomal bins. Thus scaled, a diploid ccfDNA NIPT profile is normally centered on 1, rather than on the number of chromosomes (2 in the diploid case). Deviations from 1 suggest either maternal or fetal CNVs or aneuploidies.

To successfully extract biological features (subchromosomal CNVs and whole-chromosome aneuploidies, either fetal or maternal) from a scaled NIPT ccfDNA count profile, one first needs to address technical sources of unevenness, including mappability, GC bias, and stochastic errors due to counting statistics. To reduce these sources of error, we model raw bin count n as a Poisson random variable with the mathematical expectation given by the following expression:

$$𝔼 ( n \vert S , M , F , f , g , m , L , Q ) = \frac{S}{2} [ ( 1 - f ) M + f F ] mG ( g , L , Q , {g}_{0} ) = \frac{S}{2} WmG ( g , L , Q , {g}_{0} ) \qquad ( 22 )$$

Eq. 22 is similar to the expression used for mean raw bin counts in the scDNA case. The terms g (GC content per bin), L (linear GC bias coefficient), Q (quadratic GC bias coefficient), g₀ (GC reference point), and m (mappability) have the same meaning as in the scDNA case. The parabola G(g,L,Q,g₀) describing the GC bias is also the same as in the scDNA case. The scale S in Eq. 22 is divided by two, reflecting the ccfDNA convention to scale diploid genomes to one. The main difference is that the single cell ploidy P is here replaced with a combination of the maternal ploidy M and fetal ploidy F, with the mixture coefficients defined by the fetal fraction f. Unlike the integer-valued single cell ploidy P, the combination of maternal and fetal ploidies W=(1−f)M+fF is real-valued and takes different values in different samples, depending on the fetal fraction f.

The proposed solution to the problem of removing systematic errors constructs the likelihood as the conditional probability of observing raw count n, given the combination W of maternal and fetal ploidies, as Poisson probability with the mean given by Eq. 22.

$$ℙ ( n \vert S , M , F , f , g , m , L , Q ) = 𝒫 ( n ; λ = \frac{S}{2} WmG ( g , L , Q , {g}_{0} ) ) \qquad ( 23 )$$

The terms S, m, and G(g,L,Q,g₀) in Eq. 23 are known for each bin. The bin count n is measured. The only unknown in Eq. 23 is the combined maternal and fetal ploidy W. The term W is the biological source of variation that we want to extract from measurements. To estimate W, we apply Bayes Theorem and evaluate the posterior distribution [symbol] (W|n) by multiplying the likelihood [symbol] (n|S,W,g,m,L,Q) with the prior [symbol] (W) and dividing it with the prior for raw bin counts [symbol] (n). The choices for the two priors are the same as in the scDNA case. In some embodiments, the count prior [symbol] (n) is uniform. In some embodiments, the ploidy prior [symbol] (W) is uniform for the first bin, and then each subsequent bin is assigned a weighted mixture ploidy prior [symbol] (W), composed of two contributions: a uniform and a posterior [symbol] (W|n) obtained for the previous bin. In some embodiments, the mixture weights for the uniform and the previous posterior are 0.05 and 0.95, respectively.

In some embodiments, the posterior probability [symbol] (W|n) is evaluated for a plurality of values W ranging from 0 to max(n)/S. In some embodiments, the values of the combined ploidy W are sampled equidistantly in intervals of 10⁻³.

Once the posterior probability distribution [symbol] (n|S,W,g,m,L,Q) is evaluated for a range of representative W values in a given bin, the most likely value for the weighted ploidy W is obtained as either 1) the mean, 2) the median, or 3) the mode of the posterior [symbol] (W|n). In some embodiments, the preferred method to estimate W is to use the mean of the posterior distribution [symbol] (W|n).

The uncertainty in the estimate of the combined ploidy W can be obtained from the posterior distribution [symbol] (n|S,W,g,m,L,Q) as its spread. In some embodiments, the uncertainty is estimated as standard deviation, MAD, half-width, or inter-quartile range of [symbol] (W|n). In some embodiments, the uncertainties in W per bin are used in downstream analyses, for example when measuring fetal fraction or segmenting the normalized W profile to detect fetal and/or maternal CNVs.

E. Profile Segmentation using Fourier Coefficient Ratio

Notation

| | |
|---|---|
| 1. | CNV: Copy-number variant. |
| 2. | bin: Genomic segment. May be contiguous or disjunct. May overlap other bins, |
| | touch them without intersections, or stand in isolation from the other bins. |
| 3. | N: Number of genomic bins. |
| 4. | x: Compressed bin coordinates, obtained by multiplying each bin index i with |
| | 2π and dividing the product with the total number of bins N: xᵢ = 2πi/N, |
| | where i ∈ {1, 2, . . . , N}. |
| 5. | δ: increment separating two successive values of the array x: δ = xⱼ₊₁ − xⱼ = |
| | 2π/N, where j ∈ {1, 2, . . . , N − 1} |
| 6. | W: Array of raw bin counts. i-th element Wᵢ of the array W contains the count |
| | of sequenced DNA fragments (reads) that align within the genomic bin i. |
| 7. | Y: Array of pre-processed bin counts, derived from raw bin counts W using any |
| | combination of pre-processing steps (scaling, filtering, and/or normalization). |
| | Each pre-processing step accounts for one or more factors affecting profile elevation and |
| | uniformity, including but not limited to overall coverage depth, |
| | coverage breadth, mappability, repeatability, N-base content, and GC content. |
| 8. | Yᵢ: i-th element of the pre-processed bin count array Y, where i runs from 1 to N. |
| 9. | [symbol] : Scaled array of pre-processed bin counts, obtained by dividing the elements |
| | of the pre-precessed bin count array Y with its sum δΣᵢ₌₁^{N} Yᵢ. |
| 10. | ${𝓎}_{i} : i - th element of the scaled array of bin counts 𝓎 : {𝓎}_{i} = \frac{{Y}_{i}}{δ {\sum }_{j = 1}^{N} {Y}_{j}}$ |
| 11. | ${a}_{0} : Zero - order Fourier coefficient of the scaled bin count array 𝓎 : {a}_{0} = \frac{δ}{2 π} {\sum }_{i = 1}^{N} {𝓎}_{i} = \frac{1}{2 π}$ |
| 12. | K: Truncation order of Fourier expansion of the scaled bin count array [symbol] . |
| 13. | aₖ: k-th cosine Fourier coefficient of the scaled bin count array [symbol] , where k ∈ |
| | $\{ 1 , 2 , … , K \} . {a}_{k} is evaluated as {a}_{k} = \frac{δ}{π} {\sum }_{i = 1}^{N} {𝓎}_{i} cos ( {kx}_{i} )$ |
| 14. | bₖ: k-th sine Fourier coefficient of the scaled bin count array [symbol] , where k ∈ |
| | $\{ 1 , 2 , … , K \} . {b}_{k} is evaluated as {b}_{k} = \frac{δ}{π} {\sum }_{i = 1}^{N} {𝓎}_{i} sin ( {kx}_{i} )$ |
| 15. | F(K): Fourier coefficient ratio, evaluated as the sum of squares of sine and |
| | cosine Fourier coefficients aₖ and bₖ (with k running from 1 to truncation order |
| | K), divided by square of the zero-order Fourier coefficient a₀: F(K) = |
| | $\frac{1}{{a}_{0}^{2}} {\sum }_{k = 1}^{K} ( {a}_{k}^{2} + {b}_{k}^{2} ) . As implied by notation , F ( K ) is a function of the Fourier$ |
| | truncation order K. |
| 16. | z: Array of successive differences of the bin count array [symbol] . |
| 17. | zᵢ: i-th element of the array of successive differences z, evaluated from elements |
| | of scaled bin counts [symbol] as follows: zᵢ = [symbol] ᵢ₊₁ − [symbol] ᵢ, with i ∈ {1, 2, . . . , N − 1}. |
| 18. | D: Differential variance, obtained as variance of the array of successive differences |
| | z: D = [symbol] ar(z). |
| 19. | G(K): Fourier coefficient ratio F(K), divided by the differential variance D: |
| | G(K) = F(K)/D. Similarly to F(K), G(K) is a function of the Fourier |
| | truncation order K. |

To facilitate copy-number analyses, genomic coordinates are segregated into an ordered sequence of segments called genomic bins, for convenience also referred to simply as bins. Genomic bins can be topologically contiguous or consist of disjunct sets of genomic coordinates. Successive bins may have non-zero overlap, they may touch one another without overlapping, or may be separated by genomic stretches of varying lengths. The same is true for bins that are not immediate neighbors in the ordered sequence. Genomic bins may be of equal or varying lengths. Typical bin sizes can range from several base-pairs (bp) to multiple kilobases (kb), including bin sizes as large as a few megabases (MB). Examples of bin sizes include 5 kb, 10 kb, 20 kb, 50 kb, 100 kb, 200 kb, 500 kb, 1 Mb, 2 Mb, as well as any positive integer values within or outside this range. The total number of bins is denoted with symbol N.

The sequence of genomic bins is associated with bin indices and with the derived sequence of N real numbers x, reflecting bin indices and thus indirectly also reflecting genomic coordinates of individual bins. The coordinates x are compressed into the segment [0, 2π]. For that reason, this disclosure refers to x as “compressed bin coordinates.” In the special case of contiguous, non-overlapping bins of equal lengths, the sequence x contains elements spanning the range from 0 to 2π and evaluated as follows:

xᵢ=2πi/N,i∈{1,2, . . . ,N} (24)

The distance between two successive compressed bin coordinates x is labeled δ and evaluated as follows.

δ=xⱼ₊₁−xⱼ=2π/N,j∈{1,2, . . . ,N−1} (25)

Further discussions assume non-overlapping, contiguous bins of equal lengths. Extensions from Eq. 24 to the more general cases of overlapping or disjunct bins, possibly having varying lengths, is straightforward to those skilled in art and thus wholly included in the current disclosure.

Typically, each chromosome or assembly contig is assigned a separate sequence of genomic bins. Alternatively, the entire genome may be represented by a single sequence of bins, effectively merging binning partitions of individual chromosomes or assembly contigs. A single bin may comprise genomic segments from the same chromosome or assembly contig or may include genomic coordinates from multiple different chromosomes or assembly contigs.

To detect copy-number variants (CNVs) in a given genome, the starting point is to sequence fragmented genomic DNA. Next, one aligns the resulting sequences of genomic fragments to the reference genome. Each bin is assigned the count of sequenced reads whose alignment coordinates fall within that bin. The symbol W denotes the resulting array of raw bin counts.

Prior to detecting copy number variants, the raw bin counts W are subject to various pre-processing steps, including but not limited to scaling with respect to read counts per bin per chromosome, normalization with respect to bin mappability, filtering based on bin mappability, repetitiveness, N-base content, or GC content, and normalization with respect to GC content per bin. Various approaches to these pre-processing steps are elaborated elsewhere in this document. The current discussion assumes that the various scaling, filtering, and normalization steps have already been applied to raw bin counts W and that the resulting pre-processed bin counts Y are minimally affected by technical sources of variation. As such, the counts Y are deemed ready to be inspected for presence of biological copy-number variants.

For convenience, pre-processed bin counts Y are scalled with respect to their overall number to yield scaled bin counts [symbol] :

$${y}_{i} = \frac{{Y}_{i}}{δ {\sum }_{j = 1}^{N} {Y}_{j}} \qquad ( 26 )$$

To identify copy-number variant edges and ploidy values, we employ a uniformity statistic Q whose value reflects uniformity of the profile. Ideally, a uniform (homogeneous) profile should give rise to uniformity statistic values q that follow a well-defined, narrow probability distribution. We denote the uniform profile case as null-hypothesis H₀ and the resulting distribution of the uniformity statistic as [symbol] (q). Non-uniform profiles, consisting of two or more segments at different elevations, correspond to the alternative hypothesis H₁. If the alternative hypothesis is true, it should result in a distribution [symbol] (q) that minimally overlap with the null distribution [symbol] ₀(q).

Copy-number detection based on the uniformity statistic Q employs the segmentation procedure roughly outlined as follows. The segmentation starts by evaluating the uniformity statistic Q for the entire profile or its selected portion. If the resulting value q is well within the null distribution [symbol] ₀(q), the profile (or its selected portion) is uniform and segmentation is finished. Otherwise, the current segment is iteratively divided (either by bisection or using an optimized procedure, as elaborated below) and tested for uniformity. If uniform, the subdivisions are flagged as such. Non-uniform segments are further subdivided until all subdivisions are uniform or a pre-defined resolution limit is reached. Finally, neighboring uniform segments of same elevation are merged. The end result is a set of profile segments with uniform elevations.

Segment elevations in a segmented profile may have integer or real elevations.

To effectively perform segmentation, the procedure needs an optimal choice of uniformity statistic Q. The inventor of the currently disclosed procedure has previously described two possible choices for Q: Jensen Shannon entropy and Differential Variance Ratio. Both successfully detect CNVs by segmenting genomic profiles into regions of uniform elevation. The current disclosure describes two novel and superior alternatives: Fourier Coefficient Ratio F(K) and Fourier Coefficient Ratio divided by Differential Variance G(K), further explained below.

Before defining the two newly proposed statistics and explicitly stating the expressions to evaluate them, we first introduce notation and definitions for the Fourier transform of the scaled profile [symbol] . Given the array [symbol] and the increment δ (cf. Eq. 25), the zero order Fourier coefficient a₀ is:

$${a}_{0} = \frac{δ}{2 π} {\sum }_{i = 1}^{N} {y}_{i} = \frac{1}{2 π} \qquad ( 27 )$$

The integer K (K≥1) represents the truncation order of the Fourier expansion of the scaled bin count array [symbol] . The k-th (1≤k≤K) cosine coefficient of the Fourier expansion is aₖ, evaluated as follows.

$${a}_{k} = \frac{δ}{π} {\sum }_{i = 1}^{N} {y}_{i} cos ( k {x}_{i} ) , k ∈ \{ 1 , 2 , … , K \} \qquad ( 28 )$$

The corresponding k-th sine Fourier coefficient bₖ (1≤k≤K) is evaluated as follows.

$${b}_{k} = \frac{δ}{π} {\sum }_{i = 1}^{N} {y}_{i} sin ( k {x}_{i} ) , k ∈ \{ 1 , 2 , … , K \} \qquad ( 29 )$$

The first proposed candidate for the uniformity statistic Q is the Fourier Coefficient Ratio, denoted by the symbol F(K) and evaluated as the sum of squares of sine and cosine Fourier coefficients aₖ and bₖ (with k running from 1 to truncation order K), divided by the square of the zero order Fourier coefficient a₀.

$$F ( K ) = \frac{1}{{a}_{0}^{2}} {\sum }_{k = 1}^{K} ( {a}_{k}^{2} + {b}_{k}^{2} ) \qquad ( 30 )$$

The formulation of the statistic F(K) according to Eq. 30 is motivated by the fact that, ideally, a₀ is the only non-zero Fourier coefficient in a uniform profile. In reality, due to noise in the scaled bin counts [symbol] , higher-order Fourier coefficients aₖ and bₖ (1≤k≤K) may slightly deviate from zero. The resulting estimate of F(K) is a therefore a random variable with narrow distribution [symbol] ₀(F) centered close to zero, satisfying one of the fundamental criteria for the optimal statistic Q.

The value of the Fourier Coefficient Ratio F(K) depends on the choice of the truncation order K. Eq. 30 therefore represents a family of related statistics, one for each K (K≥1). The optimal choice of K depends on the profile length, desired resolution of CNV detection, and the amount of noise. In practice, K may take values 3, 5, 7, or any value including but not limited to integers from 3 to 10.

When evaluated according to Eq. 30 on a non-uniform profile [symbol] , the statistic F(K) follows an alternative-hypothesis distribution [symbol] ₁(F) distinct from [symbol] ₀(F). The exact location and spread of [symbol] ₁(F), as well as the amount of overlap between the distributions [symbol] ₀(F) and [symbol] ₁(F) depend on the amount of noise within uniform segments of the (possibly) non-uniform profile [symbol] , the lengths of the segments, and the differences among their elevations. Larger distances separating segment elevations and increased fractional lengths (closer to π on the x scale, or to ½ on the scale from 0 to 1) of stretches placed at different elevations result in larger gaps between [symbol] ₀(F) and [symbol] ₁(P). On the other hand, larger variance within uniformly elevated segments reduces the gap. In most practically relevant cases, the gap is sufficient for the segmentation to achieve high performance levels (low false positive and false negative rates).

As an example, consider a profile consisting of two segments at elevations E and E+Δ. Assume that the first segment occupies x coordinates from origin to f, with the second placed between f and 2π. The Fourier coefficients aₖ are then:

$${{{a}_{k} = \frac{E}{π} {\int }_{0}^{f} cos ( kx ) dx + \frac{E + Δ}{π} {\int }_{f}^{2 π} cos ( kx ) dx = \frac{E}{k π} sin ( kx ) \vert}_{0}^{f} + \frac{E + Δ}{k π} sin ( kx ) \vert}_{f}^{2 π} = \frac{E}{k π} sin ( kf ) - \frac{E + Δ}{k π} sin ( kf ) = \frac{- Δ}{k π} sin ( kf )$$

Similarly, sine Fourier coefficients bₖ are:

$${{{b}_{k} = \frac{E}{π} {\int }_{0}^{f} sin ( kx ) dx + \frac{E + Δ}{π} {\int }_{f}^{2 π} sin ( kx ) dx = \frac{- E}{k π} cos ( kx ) \vert}_{0}^{f} - \frac{E + Δ}{k π} cos ( kx ) \vert}_{f}^{2 π} = \frac{E}{k π} [ 1 - cos ( kf ) ] - \frac{E + Δ}{k π} [ 1 - cos ( kf ) ] = \frac{- Δ}{k π} [ 1 - cos ( kf ) ]$$

Notice:

$${a}_{k}^{2} + {b}_{k}^{2} = {( \frac{Δ}{k π} )}^{2} \{ {sin}^{2} ( kf ) + {[ 1 - cos ( kf ) ]}^{2} \} = 2 {( \frac{Δ}{k π} )}^{2} [ 1 - cos ( kf ) ]$$

The resulting expression for F(K) is then:

$$F ( K ) = \frac{1}{{a}_{0}^{2}} {\sum }_{k = 1}^{K} ( {a}_{k}^{2} + {b}_{k}^{2} ) = 4 {πΔ}^{2} {\sum }_{k = 1}^{K} \frac{1}{k} [ 1 - cos ( kf ) ] \qquad ( 31 )$$

The dominant term in RHS of Eq. 31 corresponds to k=1. It increases quadratically with of the gap between elevations Δ. In addition, it reaches maximum at f=r and vanishes at f=0 or f=2π, in accord with intuitive expectations. Higher-order terms (kl) additionally strengthen this trend. This example illustrates that the uniformity statistic F(K) exhibits the desired behavior when alternative hypothesis H₁ is true.

While both null-hypothesis distribution [symbol] ₀(F) and alternative hypothesis distribution [symbol] ₁(F) of F(K) show desirable features ( [symbol] ₀(F) is tightly concentrated near zero, while [symbol] ₁(F) is shifted to the right from [symbol] ₀(F) with a distance between the two distributions increasing as the gap Δ and the affected fraction f increase), they also both present the same difficulty: the mean values of both [symbol] ₀(F) and [symbol] ₁(F) depend on the variance within the uniform profile segments. Two otherwise identical flat profiles with different amounts of noise result in different [symbol] ₀(F) distributions, with their mean, median, and mode values shifting in accordance with the extent of spread in each profile. The same is true for [symbol] ₁(F). F(K) is not a pivotal quantity. It would be more convenient if both null-hypothesis and alternative hypothesis distributions of the uniformity statistic Q were independent of the variance of uniform segments of the profile.

The solution is to convert the non-pivotal uniformity statistic F(K) to a pivotal uniformity statistic by dividing it with another statistic which behaves the same as F(K) when the profile is flat (and, in particular, exhibits the same dependence on the variance of the flat profile), but unlike F(K) does not “sense” the presence of non-uniformity in the profile (segments at different elevations). One such statistic that is sensitive to variance in flat portions of the profile but ignores changes in elevation is Differential Variance, D, defined below.

Before defining the Differential Variance D, we first introduce the successive differences profile z. The array z contains N−1 elements derived from the scaled profile [symbol] by subtracting [symbol] 's neighboring elements from one another:

zᵢ= [symbol] ᵢ₊₁− [symbol] ᵢ,i∈{1,2, . . . ,N−1} (32)

The array z can be viewed as the differential of [symbol] : z=(d [symbol] /dx)δ. The Differential Variance statistic D is defined as the variance of the successive differences profile z:

D= [symbol] ar(z) (33)

If the original profile [symbol] is flat, D is directly proportional to the variance of the [symbol] , with the proportionality constant of 2:

D=2 [symbol] ar( [symbol] ) (34)

When the profile [symbol] consists of two or more segments at different elevations, the Differential Variance D is proportional to the variance of the individual uniform segments within the non-uniform profile [symbol] . The operation of taking successive differences flattens the elevation, so that a non-uniform profile [symbol] gives almost the same value of D as another uniform profile [symbol] with the same within-segment variance. The changes in elevation in the profile [symbol] , if they exist, are reflected in z as upward or downward spikes, depending on whether the elevation increases or decreases at a given location. The spikes in z can potentially affect the value of D. To avoid this undesired effect, extreme values of |z| can be trimmed prior to evaluating D.

When the profile [symbol] is flat, the uniformity statistic F(K) is proportional to Differential Variance D, and both are proportional to the variance of [symbol] . Thus, dividing F(K) with D will result in a statistic that is independent on noise when the profile is flat. We therefore define a new uniformity statistic G(K), representing the Fourier Coefficient Ratio F(K) normalized with respect to D:

G(K)=F(K)/D (35)

Similarly to F(K), the statistic G(K) is a function of the Fourier truncation order K. Also, as in the case of F(K), the normalized uniformity statistic G(K) estimated from a uniform profile [symbol] is tightly distributed close to the origin. Unlike F(K), the mean and spread of [symbol] ₀(G) do not depend on the noise in the profile [symbol] , meaning that G(K) is pivotal. The use of the redefined uniformity statistic G(K) is therefore preferred to F(K).

F. Single Cell Clustering using Fourier Coefficient Ratio

In a heterogeneous mixture of cells with different copy-number profiles, subsets of cells are usually grouped together based on the degree of similarity of their copy number profiles. The motivation for grouping is 1) to improve signal to noise by using collective information from measurements collected on multiple similar cells, and 2) to reconstruct the evolutionary path leading to the present heterogeneous state. The grouping is achieved using one of multiple existing clustering techniques. Two main aspects of cell clustering include: a) clustering algorithm, and b) dissimilarity (or similarity) measure. The current disclosure proposes a new dissimilarity measure: Fourier Coefficient Ratio of Cell Profile Difference.

The following procedure summarizes the use of Fourier Coefficient Ratio to quantify the degree of dissimilarity between two copy number profiles:
1. Generate raw read count profiles.
2. Evaluate GC bias coefficients separately for each profile, as described on page 15.
3. Normalize each raw count profile with respect to mappability and its GC bias, as described on page 26.
4. Evaluate scale separately for each profile, as described on page 32.
5. Divide each normalized copy number profile with its scale.
6. For each pair of profiles, subtract the profiles. The choice of which profile figures in the difference with positive or negative sign is arbitrary.
7. To avoid division by small numbers and improve numerical stability, add a non-zero constant to the difference evaluated in the previous step.
8. Evaluate Fourier Coefficient Ratio, divided by Differential Variance Ratio, as described on page 43.

The resulting value of the Fourier Coefficient Ratio divided by Differential Variance Ratio represents the dissimilarity for the given pair of copy number profiles. When two profiles are similar, this metric takes values close to zero. Very dissimilar profiles give rise to high FCR values, which quickly increase as the dissimilarity between profiles increases.

In some embodiments, Fourier Coefficient Ratio without division with Differential Variance Ratio is used as the dissimilarity metric. In some embodiments, Jensen-Shannon entropy is used as the dissimilarity metric. In some embodiments, Differential Variance Ratio is used as the dissimilarity metric.

G. Quantification of Impurities in cfDNA Using Minor Allele Frequencies

Notation

| | |
|---|---|
| 1. | R: Reference allele in sample genotype. |
| 2. | A: Alternate allele in sample genotype. |
| 3. | G: Sample genotype. Can take values RR, RA, or AA |
| 4. | r: Reference allele in contamination genotype. |
| 5. | a: Alternate allele in contamination genotype. |
| 6. | _: Absence of any contamination alleles. |
| 7. | g: Contamination genotype. Can take values rr, ra, aa, or _. The value _ |
| | represents absence of contamination. |
| 8. | f: Fraction of DNA originating from the contamination, satisfying f ∈ [0, 1/2]. |
| 9. | p_{C}: Prior probability that the sample is contaminated. |
| 10. | p_{A}: Population frequency of the alternate allele. |
| 11. | δ(x): Dirac delta function. |
| 12. | [symbol] (x; l, u): Continuous uniform distribution for random variable x, with domain |
| | (support) ranging from the lower bound l to the upper bound u. |
| 13. | n_{R}: Count of reference alleles at a given locus. |
| 14. | n_{A}: Count of alternate alleles at a given locus. |
| 15. | [symbol] (x; α, β): Beta distribution for random variable x, with parameters α and β. |
| 16. | [symbol] (g): Prior probability of contamination. Equals 1 − p_{C} when g = _ , or p_{C} |
| | otherwise. |
| 17. | [symbol] (G, g): Prior probability for the given combination of sample and contamination |
| | genotypes G and g. |
| 18. | ψ: Alternate allele fraction. |
| 19. | [symbol] (ψ\|n_{A}, n_{R}): Conditional probability of alternate allele frequency ψ, given the |
| | measured allele counts n_{A} and n_{R}. [symbol] (ψ\|n_{A}, n_{B}) is expressed in terms of |
| | contamination level f as composite function: [symbol] (ψ(f)\|n_{A}, n_{B}). |
| 20. | J: Jacobian J = df/dψ). |
| 21. | NaN: Not a number. |
| 22. | v_{A}, v_{R}: Additive corrections for drop-out alleles. |
| 23. | μ_{A}, μ_{R}: Multiplicative corrections for drop-out alleles. |
| 24. | ϵ_{A}, ϵ_{R}: Additive corrections for drop-in alleles. |
| 25. | η_{A}, η_{R}: Multiplicative corrections for drop-in alleles. |
| 26. | c: Lower bound on allele counts. |
| 27. | t, T: Lower and upper bounds on total counts per locus. |

Problem Formulation

Given measured raw allele counts across a panel of preselected SNP loci in a human sample, estimate contamination level, along with the associated uncertainty.

Genotypes and Allele Frequencies

Notation: uppercase symbols R and A represent reference and alternate alleles at a given locus in the main sample. Lowercase r and a stand for reference and alternate alleles in contaminating DNA material, if present. Two consecutive underscores denote absence of contamination. The following twelve scenarios are possible:

TABLE 0.1
Genotype Combinations and Probabilities.

| A | B | C | D | E | F | G | H |
|---|---|---|---|---|---|---|---|
| 1 | RR | — | 0 | (1 − p_{A})² | 1 − p_{C} | δ(ƒ) | 0 |
| 2 | RA | — | ½ | 2(1 − p_{A})p_{A} | 1 − p_{C} | δ(ƒ) | 0 |
| 3 | AA | — | 1 | p_{A}² | 1 − p_{C} | δ(ƒ) | 0 |
| 4 | RR | rr | 0 | (1 − p_{A})² | p_{C}(1 − p_{A})² | [symbol] (ƒ; 0, ½) | NaN |
| 5 | RR | ra | ƒ/2 | (1 − p_{A})² | 2p_{C}p_{A}(1 − p_{A}) | [symbol] (ƒ/2; n_{A} + 1, n_{R} + 1) | 2 |
| 6 | RR | aa | ƒ | (1 − p_{A})² | p_{C}p²_{A} | [symbol] (ƒ; n_{A} + 1, n_{R} + 1) | 1 |
| 7 | RA | rr | ½(1 − ƒ) | 2(1 − p_{A})p_{A} | p_{C}(1 − p_{A})² | [symbol] ((1 − ƒ)/2; n _{A} − 1, n_{B} − 1) | −2 |
| 8 | RA | ra | ½ | 2(1 − p_{A})p_{A} | 2p_{C}p_{A}(1 − p_{A}) | [symbol] (ƒ; 0, ½) | NaN |
| 9 | RA | aa | ½(1 + ƒ) | 2(1 − p_{A})p_{A} | p_{C}p_{A}² | [symbol] ((1 + ƒ)/2; n_{A} + 1, n_{B} + 1) | 2 |
| 10 | AA | rr | 1 − ƒ | p_{A}² | p_{C}(1 − p_{A})² | [symbol] (1 − ƒ; n_{A} + 1, n_{R} + 1) | −1 |
| 11 | AA | ra | 1 − ƒ/2 | p_{A}² | 2p_{C}p_{A}(1 − p_{A}) | [symbol] (1 − ƒ/2; n_{B} +1 , n_{B} − 1) | −2 |
| 12 | AA | aa | 1 | p_{A}² | p_{C}p_{A}² | [symbol] ( ƒ; 0, ½) | NaN |

Column names:
1. A: Scenario
2. B: Sample Genotype G
3. C: Contamination Genotype g
4. D: Alternate Allele Frequency ψ as a Function of Contamination Level ƒ and genotypes G and g: ψ = ψ(ƒ; G, g)
5. E: Sample Genotype Prior [symbol] (G)
6. F: Contaminant Genotype Prior [symbol] (g)
7. G: Conditional Probability [symbol] (ψ(ƒ; G, g)|n_{A}, n_{R})
8. H: Jacobian J = dƒ/dψ
The second and third columns list sample and contamination genotypes in upper-and lowercase letters, respectively. Column D (Alternate Allele Frequency) shows relationships between observed alternate allele frequencies and contamination level ƒ. All scenarios where allele frequency does not depend on contamination ƒ are uninformative.

Table 0.1 lists prior probabilities [symbol] (G) and [symbol] (g) in columns E-F for all 12 scenarios.

The conditional probability of contamination level values f, given the observed allele counts n_{A} and n_{R}, is obtained by summing up contributions from all twelve scenarios:

$$ℙ ( f \vert {n}_{A} , {n}_{R} ) = {\sum }_{G , g} \frac{ℙ ( G ) ℙ ( g )}{\vert J ( f , ψ ) \vert} ℙ ( ψ ( f ; G , g ) \vert {n}_{A} , {n}_{R} ) \qquad ( 36 )$$

To enhance the predictive power of Eq. 36, we introduce non-negative weights [symbol] (G,g), satisfying the normalization conditions:

$$w ( G , g ) \geq 0 , ∀ G , ∀ g \qquad ( 37 )$$

$${\sum }_{G , g} w ( G , g ) = 1 \qquad ( 38 )$$

In some embodiments all weights [symbol] (G,g) are equal. In some embodiments, selected combinations of sample and contamination genotypes G,g are assigned higher weights than other scenarios. In some embodiments, uninformative scenarios (RR.rr, RA.ra, and AA.aa) are assigned lower weights than the remaining, informative scenarios. In some embodiments, scenarios with heterozygous contamination genotypes are assigned higher weights than other scenarios. In some embodiments, scenarios with homozygous contamination genotypes are assigned higher weights than other scenarios. In some embodiments, an arbitrary subset of scenarios are favored by assignment of higher weights.

The introduction of weights transforms Eq. 36 as follows.

$$ℙ ( f \vert {n}_{A} , {n}_{R} ) = {\sum }_{G , g} \frac{ℙ ( G ) ℙ ( g )}{\vert J ( f , ψ ) \vert} ℙ ( ψ ( f ; G , g ) \vert {n}_{A} , {n}_{R} ) w ( G , g ) \qquad ( 39 )$$

The fraction of contamination f₀ is estimated as the central tendency of the posterior probability distribution [symbol] (f|n_{A},n_{R}). In some embodiments, the estimate f₀ is obtained as the mode (location of the maximum) of the posterior [symbol] (f|n_{A},n_{R}):

f₀=argmax( [symbol] (f|n_{A},n_{R})) (40)

In some embodiments, the estimate f₀ is obtained as the mean of the posterior [symbol] (f|n_{A},n_{R}):

f₀=∫₀^{1/2} [symbol] (f|n_{A},n_{R})fdf (41)

In some embodiments, the estimate f₀ is obtained as the median of the posterior [symbol] (f|n_{A},n_{R}), ie. as the solution of the following integral equation:

∫₀^{f}^{₀} [symbol] (f|n_{A},n_{R})df=∫_{f}_{₀}^{1/2} [symbol] (f|n_{A},n_{R})df (42)

The uncertainty in f₀ is estimated as the spread of the posterior probability distribution [symbol] (f|n_{A},n_{R}). In some embodiments, the uncertainty in f₀ is obtained as the variance of the posterior [symbol] (f|n_{A},n_{R}):

[symbol]

ar(f₀)=∫₀^{1/2} [symbol] (f|n_{A},n_{R})f²df−(∫₀^{1/2} [symbol] (f|n_{A},n_{R})t)fdf² (43)

In some embodiments, the uncertainty in f₀ is obtained as the half-width of the posterior [symbol] (f|n_{A},n_{R}). In some embodiments, the uncertainty in f₀ is obtained as the MAD (median absolute deviation) of the posterior [symbol] (f|n_{A},n_{R}).

In some embodiments, uninformative loci (those with reference allele counts too low, or alternate allele counts too low) are filtered out. In some embodiments, reference or alternate allele counts are deemed too low if they are lower than a user-defined cutoff c. In some embodiments, loci with n_{A}<c or n_{R}<c are filtered out. In some embodiments, the cutoff c is set to 1.

In some embodiments, loci with too few total counts are filtered out. In some embodiments, a user-defined lower limit t on total allele counts is imposed. In some embodiments, the lower limit t is 10, 20, 30, 40, 50, 60, 70, 80, 90, 100, or any value in between. In some embodiments, loci with n_{A}+n_{R}<t are filtered out.

In some embodiments, loci with too many total counts are filtered out. In some embodiments, a user-defined upper limit T on total allele counts is imposed. In some embodiments, the upper limit T is 1000, 2000, 3000, 4000, 5000, 6000, 7000, 8000, 9000, 10000, or any value in between. In some embodiments, loci with n_{A}+n_{R}>T are filtered out.

In some embodiments, loci with drop-out alleles are corrected by introducing additive factors ν_{A} and ν_{R} to allele counts. In those embodiments, posterior probabilities are evaluated by replacing n_{A} and n_{R} with n_{A}+ν_{A},n_{R}+ν_{R}, respectively.

In some embodiments, loci with drop-out alleles are corrected by introducing multiplicative factors μ_{A} and μ_{R} to allele counts. In those embodiments, posterior probabilities are evaluated by replacing n_{A} and n_{R} with n_{A}μ_{A},n_{R}μ_{R}, respectively.

In some embodiments, loci with drop-in alleles are corrected by introducing additive factors ϵ_{A} and ϵ_{R} to allele counts. In those embodiments, posterior probabilities are evaluated by replacing n_{A} and n_{R} with n_{A}−ϵ_{A},n_{R}−ϵ_{R}, respectively.

In some embodiments, loci with drop-in alleles are corrected by introducing multiplicative factors η_{A} and η_{q} to allele counts. In those embodiments, posterior probabilities are evaluated by replacing n_{A} and n_{R} with n_{A}η_{A},n_{R}η_{R}, respectively.

In some embodiments, background noise is estimated by counting alternate alleles at loci that are likely homozygous for reference allele, or by counting reference alleles at loci that are likely homozygous for alternate allele.

In some embodiments, locus-specific and sample-specific GC bias is corrected by using additive or multiplicative correction. In some embodiments, the GC correction is a linear or quadratic function of the GC content of the region surrounding the locus. In some embodiments, the GC correction is also a function of a sample-specific coefficient.

In some embodiments, reference bias is corrected by applying an additive or multiplicative term to reference and/or alternate allele counts.

In some embodiments, the conditional probability [symbol] (f|n_{A},n_{R}) is modeled using Poisson distribution, rather than beta distribution, following the same approach as the model described in Section H. (Measurement of Fetal Fraction in cfDNA Using Minor Allele Frequencies).

Once the continuous distributions of contamination level is constructed for all loci in a sample, the distributions are logarithmically transformed and added together to yield log(P) profile over all loci. The log(P) profile is then further examined to estimate the contamination level per sample, as well as the associated uncertainty. Mean, mode, median, variance, and MAD for the combined continuous probability distribution of the contamination are obtained using standard procedures known to those skilled in the art.

H. Measurement of Fetal Fraction in cfDNA Using Minor Allele Frequencies

Notation

| | |
|---|---|
| 1. | cfDNA: Cell-free DNA from maternal plasma. |
| 2. | ƒ: Fetal fraction in cfDNA. |
| 3. | R: Reference allele in maternal DNA. |
| 4. | A: Alternate allele in maternal DNA. |
| 5. | r: Reference allele in fetal DNA. |
| 6. | a: Alternate allele in fetal DNA. |
| 7. | M: Maternal genotype. Allowed values: RR, RA, or AA. |
| 8. | F: Fetal genotype. Allowed values: rr, ra, or aa, with the additional constraint |
| | that at least one allele must match one of maternal alleles. |
| 9. | p_{A}: Population frequency of the alternate allele. |
| 10. | w_{M}(M, p_{A}): Prior probability of the maternal genotype for a given scenario, a |
| | function of the maternal genotype and the population |
| | frequency of the alternate allele p_{A}. |
| 11. | w_{F}(M, F, p_{A}): Prior probability of the fetal genotype for a given scenario, a |
| | function of the scenario (M, F) and the population frequency p_{A}. |
| 12. | [symbol] (ƒ): Prior distribution of fetal fraction values. |
| 13. | [symbol] (ƒ; α, β): Beta distribution of continuous random variable ƒ ϵ [0, 1], with |
| | shape parameters α and β. |
| 14. | α_{F}, β_{F}: Shape parameters α and β for the prior |
| | distribution of fetal fraction values. |
| 15. | N: Expected total read count for a single cfDNA NIPT sample at a given SNP |
| | locus. Estimated as a fraction of the total observed read count across the entire |
| | panel, with locus-specific proportionality constant. |
| 16. | n_{R}: Reference allele counts for a given locus. |
| 17. | n_{A}: Alternate allele counts for a given locus. |
| 18. | [symbol] (n_{R}, n_{A}): Prior distribution of observed reference |
| | and alternate allele counts for a given locus. |
| 19. | [symbol] (n_{R}, n_{A}\|ƒ): Conditional probability of observing n_{R} reference allele counts and |
| | n_{A} alternate allele counts at a given locus, given |
| | fetal fraction ƒ. Also referred to as likelihood. |
| 20. | [symbol] (ƒ\|n_{R}, n_{A}): Posterior probability of fetal fraction ƒ, given observed reference |
| | and alternate allele counts n_{R} and n_{A}, respectively. |
| 21. | [symbol] (n, λ): Poisson distribution of non-negative |
| | integer random variable n with mean λ. |

### Overall Workflow

This section describes a procedure to estimate fetal fraction in circulating cell-free DNA from maternal plasma based on reference and alternate allele counts in a set of preselected SNP loci. The panel of SNPs is selected such that all loci have significant alternate allele frequency in the population. As a result, it is likely that a NIPT sample will have sufficient number of informative loci, where either the mother is homozygous and the fetus heterozygous or the fetus is homozygous and the mother heterozygous.

The approach proposed here constructs the posterior probability of fetal fraction f for a single locus [symbol] (f|n_{R},n_{A}), conditioned on the observed reference and alternate allele counts n_{R} and n_{A} for that locus. Assuming independent loci, posterior distributions from different loci across the SNP panel are multiplied together to yield the final combined posterior distribution [symbol] (f|{n_{A},n_{B}}), where the set {n_{A},n_{B}} denotes measured allele counts for all loci. The fetal fraction estimate is obtained as the average, median, or mode of the posterior distribution [symbol] (f|{n_{A},n_{B}}). The uncertainty in the estimate is derived from the variance of [symbol] (f|{n_{A},n_{B}}).

Fetal Fraction Prior

The prior distribution for f can be either uniform on the segment from 0 to 1 (uninformative), or derived from observed fetal fraction values in a large number of NIPT samples (informative). The observed prior can be modeled using the beta distribution with shape parameters α_{F} and β_{F}:

$$ℙ ( f ) = 𝔹 ( f ; {α}_{F} , {β}_{F} ) = \frac{Γ ( {α}_{F} + {β}_{F} )}{Γ ( {α}_{F} ) Γ ( {β}_{F} )} {{f}^{{α}_{F} - 1} ( 1 - f )}^{{β}_{F} - 1} \qquad ( 44 )$$

The shape parameters α_{F} and β_{F} are chosen such that the prior [symbol] (f) has mean μ_{F} and standard deviation σ_{F} values matching the average and standard deviation values observed in a representative set of NIPT samples. The shape parameters are related to μ and σ as follows:

$${μ}_{F} = \frac{{α}_{F}}{{α}_{F} + {β}_{F}} \qquad ( 45 )$$

$${σ}_{F}^{2} = \frac{{α}_{F} {β}_{F}}{{( {α}_{F} + {β}_{F} )}^{2} ( {α}_{F} + {β}_{F} + 1 )} \qquad ( 46 )$$

Solving the above two equations for shape parameters gives the following expressions:

$${α}_{F} = {( \frac{{μ}_{F}}{{σ}_{F}} )}^{2} ( 1 - {μ}_{F} ) - {μ}_{F} \qquad ( 47 )$$

$${β}_{F} = \frac{1}{{μ}_{F}} {α}_{F} ( 1 - {μ}_{F} ) \qquad ( 48 )$$

Typical values observed in NIPT samples are μ_{F}=0.10 and σ_{F}=0.04, giving the following values for shape parameters:

α_{F}=5.525 (49)

β_{F}=49.725 (50)

Genotype Scenarios

Assuming that both maternal and fetal loci are diploid and that no de novo mutations take place in fetal DNA, there are seven possible combinations of maternal and fetal genotypes. Table 0.2 lists all possible scenarios, as well as their prior probabilities (based on the alternate allele population frequency p_{A}) and the corresponding expectation values for allele counts.

The locus-specific expected total read count N is not necessarily the same as the observed total read count at that locus (n_{R}+n_{A}). Rather than the actual per-locus count, N represents a fraction of the total read counts across the entire SNP panel, with the proportionality constant reflecting the characteristics of the locus. Various biases (allele drop-in, allele drop-out, locus drop-out, GC bias, mappability bias) can be modeled by introducing multiplicative or additive terms modulating the expected total read count N in Tbl. 0.3.

TABLE 0.2
Genotype Scenarios.

| Scenario | M | F | Weight w_{M} (M, p_{A}) | Weight w_{F} (M, F, p_{A}) |
|---|---|---|---|---|
| 1 | RR | rr | (1 − p_{A})² | (1 − p_{A})² + p_{A} (1 − p_{A}) |
| 2 | RR | ra | (1 − p_{A})² | p_{A}² + p_{A} (1 − p_{A}) |
| 3 | RA | rr | 2p_{A} (1 − p_{A}) | ½[(1 − p_{A})² + p_{A} (1 − p_{A})] |
| 4 | RA | ra | 2p_{A} (1 − p_{A} ) | ½ {[p_{A}² + p_{A} (1 − p_{A})] + |
| | | | | [(1 − p_{A})² + p_{A} (1 − p_{A})]} |
| 5 | RA | aa | 2p_{A} (1 − p_{A}) | ½ [p_{A}² + p_{A} (1 − p_{A}) |
| 6 | AA | ra | p_{A}² | (1 − p_{A})² + p_{A} (1 − p_{A}) |
| 7 | AA | aa | p_{A}² | p_{A}² + p_{A} (1 − p_{A}) |

The second column lists maternal genotypes M (uppercase; possible values: RR, RA, and AA). Third column: fetal genotypes F (lowercase; possible values: rr, ra, and aa). Fourth and fifth columns list prior maternal and fetal genotype probabilities w_{M} (M, p_{A}) and w_{F} (M, F, p_{A}) per scenario, based on population alternate allele frequency p_{A}

TABLE 0.3
Allele Count Expectations.

| Scenario | M | F | λ_{R} (M, F, f, N) | λ_{A} (M, F, f, N) |
|---|---|---|---|---|
| 1 | RR | rr | N | 0 |
| 2 | RR | ra | N (1 − f/2) | N f/2 |
| 3 | RA | rr | N (1 + f)/2 | N (1 − f)/2 |
| 4 | RA | ra | N/2 | N/2 |
| 5 | RA | aa | N (1 − f)/2 | N (1 + f)/2 |
| 6 | AA | ra | N f/2 | N (1 − f/2) |
| 7 | AA | aa | 0 | N |

The second and third columns list maternal and fetal genotypes in upper-and lowercase letters, respectively. Fourth and fifth columns show relationships between fetal fraction f and mathematical expectations λ_{R} = [symbol] (n_{R}) and λ_{A} = [symbol] (n_{A}) for reference and alternate allele counts, respectively. All scenarios where allele counts do not depend on fetal fraction f are uninformative. The symbol N stands for the expected total read count at the given locus. Locus-specific N is obtained from the total observed read count across the SNP Panel (sum over all loci), multiplied by a locus-specific factor m.

Likelihood

The joint conditional probability of observed reference and alternate allele counts n_{R} and n_{A}, given a fetal fraction value f, is a sum of terms corresponding to different scenarios 1-7. Each term in the sum is a product of the scenario's weights [symbol] _{M}(M,p_{A}) and [symbol] _{F}(M,F,p_{A}) (listed in [symbol] . 0.2) and two Poisson distributions—one for n_{R} with the mean λ_{R}(M,F,f,N)= [symbol] (n_{R}), and the other for n_{A} with the mean λ_{A}(M,F,f,N)= [symbol] (n_{A}) (Tbl. 0.3):

$$ℙ ( {n}_{R} , {n}_{A} \vert f ) = {\sum }_{( M , F )} {w}_{M} ( M , {p}_{A} ) {w}_{F} ( M , F , {p}_{A} ) 𝒫 ( {n}_{R} , {λ}_{R} ( M , F , f , N ) ) 𝒫 ( {n}_{A} , {λ}_{A} ( M , F , f , N ) ) \qquad ( 51 )$$

The symbol [symbol] (n, λ) in Eq. 51 denotes the Poisson distribution of integer random variable n with mean λ. Likelihood [symbol] (n_{R},n_{A}|f) is a function of the population frequency p_{A} and the expected total read count at the given locus N. In the interest of space, this dependence is not explicitly spelled out. The sum in Eq. 51 runs over allowed genotype pairs (M,F) tabulated in Tbls. 0.2-0.3.

In some embodiments, the summation in Eq. 51 omits uninformative terms. In some embodiments, additional non-zero positive weights are assigned to selected scenarios.

Allele Count Prior

The joint prior probability for observed allele counts n_{R} and n_{A} is obtained by integrating the product of the likelihood [symbol] (n_{R},n_{A}|f) and the fetal fraction prior [symbol] (f) over the entire domain of fetal fraction values:

[symbol]

(n_{R},n_{A})=∫₀¹ [symbol] (n_{R},n_{A}|f) [symbol] (f)df (52)

The actual value of the integral in Eq. 52 depends on the choice for the fetal fraction prior [symbol] (f) (uniform or beta-distribution). In either case, the integrals are evaluated straightforwardly using standard tabulated values. The count prior [symbol] (n_{R},n_{A}) is a function of the population frequency p_{A} and the expected total read count N. If the informative fetal fraction prior is used, the count prior is also a function of the shape parameters α_{F} and β_{F}.

Per-Locus and Overall Fetal Fraction Posterior Distributions

The desired posterior distribution of fetal fraction values f, conditioned on the observed allele counts n_{R} and n_{A} for a given locus, is obtained from the likelihood [symbol] (n_{R},n_{A}|f) and from the two priors [symbol] (f) and [symbol] (n_{R},n_{A}) by applying Bayes Theorem:

$$ℙ ( f \vert {n}_{R} , {n}_{A} ) = \frac{ℙ ( {n}_{R} , {n}_{A} \vert f ) ℙ ( f )}{ℙ ( {n}_{R} , {n}_{A} )} \qquad ( 53 )$$

The overall posterior distribution of fetal fraction values f, conditioned on all observed allele counts {n_{R},n_{A}} across the entire SNP panel, is obtained by multiplying per-locus posterior distributions [symbol] (f|n_{R},n_{A}):

$$ℙ ( f \vert \{ {n}_{R} , {n}_{A} \} ) = {\prod }_{i}^{K} ℙ ( f \vert {n}_{R} ( i ) , {n}_{A} ( i ) ) \qquad ( 54 )$$

The product index i in Eq. 54 runs over all K SNPs in the panel, assuming that the individual loci are independent. In practice, rather than multiplying per-locus posterior probabilities, the final result is obtained by summing up their logarithms, to improve numerical stability as known to those skilled in the art.

$$log ℙ ( f \vert \{ {n}_{R} , {n}_{A} \} ) = {\sum }_{i}^{K} log ℙ ( f \vert {n}_{R} ( i ) , {n}_{A} ( i ) ) \qquad ( 55 )$$

Fetal Fraction Estimate and Error

The sample's fetal fraction is estimated as the central tendency of the overall posterior distribution [symbol] (f|{n_{R},n_{A}}). In some embodiments, the estimate is obtained as the mean of [symbol] (f|{n_{R},n_{A}}). In some embodiments, the estimate is obtained as the median of [symbol] (f|{n_{R},n_{A}}). In some embodiments, the estimate is obtained as the mode of [symbol] (f|{n_{R},n_{A}}). The associated error bar is obtained from the spread of [symbol] (f|{n_{R},n_{A}}). In some embodiments, the error bar is obtained from the standard deviation of [symbol] (f|{n_{R},n_{A}}). In some embodiments, the error bar is obtained from the MAD of [symbol] (f|{n_{R},n_{A}}). In some embodiments, the error bar is obtained as the half-width of [symbol] (f|{n_{R},n_{A}}).

Quantification of contaminants, described in previous section, can be reformulated in terms of the approach described here. The main differences are that additional genotype scenarios are possible and no restrictions are imposed on minor genotype contribution. Otherwise, the same framework described here for measurement of fetal fractions also applies to measurements of contaminant levels.

I. Detection of Fetal Copy-Number Variants in cfDNA Using Minor Allele Frequencies

Notation

| | |
|---|---|
| f: | Fetal fraction. |
| N: | Expected total read count at a given SNP locus, |
| | in the absence of deletions or duplicatios |
| | in either maternal or fetal genomes. |
| R, A: | Maternal alleles (reference and alternate, |
| | respectively). |
| r, a: | Fetal alleles (reference, and alternate, respectively). |
| M: | Maternal genotype. Can take values RR, RA, or AA. |
| F: | Fetal genotype. In case of heterozygous fetal deletion, |
| | F can take values r or a. In the case of a heterozygous |
| | fetal duplication, F can take values rrr, rra, raa, or aaa. |
| w_{M}(M, p_{A}): | Probabiity of the maternal genotype M, given the |
| | population frequency p_{A} of the minor allele A. |
| w_{F}(M, F, p_{A}): | Probability of the fetal genotype F, given the |
| | population frequency p_{A}. A function of the maternal |
| | genotype M. Also depends on the origin of the |
| | duplicated or deleted allele (maternal or paternal). |
| [symbol] (f\|{n_{R}, n_{A}}): | Posterior distribution of fetal fraction values, given |
| | the observed set of reference and alternate allele |
| | counts across the entire SNP panel. |

Table 0.4 lists all possible combinations of maternal and fetal genotypes, assuming diploid mother and heterozygous fetal deletion at the locus in question. The probabilities of maternal genotypes [symbol] _{M}(M,p_{A}) and the probabilities of fetal genotypes [symbol] _{F}(M,F,p_{A}) are also listed. Both maternal and paternal allele losses are included, along with the corresponding weight factors.

Table 0.5 lists the expectation values for reference and alternate allele counts (n_{R} and n_{A}, respectively), assuming the maternal genotype is diploid and the fetus has a heterozygous deletion.

Table 0.6 lists all possible combinations of maternal and fetal genotypes, assuming diploid mother and heterozygous fetal duplication at the locus in question. The probabilities of maternal genotypes [symbol] _{M}(M,p_{A}) and the probabilities of fetal genotypes [symbol] w_{F}(M,F,p_{A}) are also listed. Both maternal and paternal allele gains are included, along with the corresponding weight factors.

TABLE 0.4
Fetal Deletion Scenarios.

| | | | Deleted | Weight w_{M} | Weight w_{F} |
|---|---|---|---|---|---|
| Scenario | M | F | Allele | (M, p_{A}) | (M, F, p_{A}) |
| 1 | RR | r | Maternal | (1 − p_{A})² | 1 − p_{A} |
| 2 | RR | a | Maternal | (1 − p_{A})² | p_{A} |
| 3 | RA | r | Maternal | 2p_{A} (1 − p_{A}) | 1 − p_{A} |
| 4 | RA | a | Maternal | 2p_{A} (1 − p_{A}) | p_{A} |
| 5 | AA | r | Maternal | p_{A}² | 1 − p_{A} |
| 6 | AA | a | Maternal | p_{A}² | p_{A} |
| 7 | RR | r | Paternal | (1 − p_{A})² | 1 |
| 8 | RR | a | Paternal | (1 − p_{A})² | 0 |
| 9 | RA | r | Paternal | 2p_{A} (1 − p_{A}) | ½ |
| 10 | RA | a | Paternal | 2p_{A} (1 − p_{A}) | ½ |
| 11 | AA | r | Paternal | p_{A}² | 0 |
| 12 | AA | a | Paternal | p_{A}² | 1 |

The first column enumerates fetal deletion scenarios. The second column lists maternal genotypes M (uppercase; possible values: RR, RA, and AA). Third column: fetal genotypes F affected by deletions (lowercase; possible values: r, a). Fourth column indicates the parental origin of the deleted allele. Fifth and sixth columns list prior maternal and fetal genotype probabilities w_{M} (M, p_{A}) and w_{F} (M, F, p_{A}) per scenario, based on population alternate allele frequency p_{A}

Table 0.7 lists the expectation values for reference and alternate allele counts (n_{R} and n_{A}, respectively), assuming the maternal genotype is diploid and the fetus has a heterozygous duplication.

Detection of fetal copy number variation at a given locus or genomic segment based on measured reference and alternate allele counts consists of several steps. The first step estimates the posterior distribution [symbol] (f|{n_{R},n_{A}}) of fetal fraction values, given observed reference and alternate allele counts at a plurality of genomic loci, as described on page 63. In some embodiments, the loci used to estimate the posterior distribution of fetal fraction values belong to genomic regions that are expected to be diploid in both the mother and the fetus. In some embodiments, the set of genomic loci used to estimate [symbol] (f|{n_{R},n_{A}}) excludes the SNP loci used to detect copy

TABLE 0.5
Fetal Deletions: Allele Count Expectations.

| Scenario | M | F | λ_{R} (M, F, f, N) | λ_{A} (M, F, f, N) |
|---|---|---|---|---|
| 1, 7 | RR | r | N (1 − f/2) | 0 |
| 2, 8 | RR | a | N (1 − f) | N f/2 |
| 3, 9 | RA | r | N/2 | N (1 − f)/2 |
| 4, 10 | RA | a | N (1 − f)/2 | N/2 |
| 5, 11 | AA | r | N f/2 | N (1 − f) |
| 6, 12 | AA | a | 0 | N (1 − f/2) |

The second and third columns list maternal and fetal genotypes in upper-and lowercase letters, respectively. Fourth and fifth columns show relationships between fetal fraction f and mathematical expectations λ_{R} = [symbol] (n_{R}) and λ_{A} = [symbol] (n_{A}) for reference and alternate allele counts, respectively. All scenarios where allele counts do not depend on fetal fraction f are uninformative. The symbol N stands for the expected total read count at the given locus. Locus-specific N is obtained from the total observed read count across the SNP panel (sum over all loci), multiplied by alocus-specific factor m.

number variants. In some embodiments, the set of genomic loci used to estimate [symbol] (f|{n_{R},n_{A}}) includes the SNP loci used to detect copy number variants. In some embodiments, fetal fraction estimation is performed assuming only diploid genotype scenarios ( [symbol] . ??). In some embodiments, fetal fraction estimation includes scenarios that account for fetal deletions and/or duplications (Tables 0.4 and 0.6). In some embodiments, the posterior distribution of fetal fractions is estimated by means other than measured allele counts. In some embodiments, the posterior distribution of fetal fraction values is obtained from whole genome resequencing data, including but not limited to chrX representation, chrY representation, fetal aneuploidy (trisomy or monosomy) of autosomal chromosomes, fetal subchromosomal CNVs, maternal subchromosomal CNVs, read count non-uniformity, or observed read lengths. In some embodiments, the posterior distribution [symbol] (f|{n_{R},n_{A}}) is replaced with the prior [symbol] (f) (see page 63).

TABLE 0.6
Fetal Duplication Scenarios.

| | | | Duplicated | Weight w_{M} | Weight w_{F} |
|---|---|---|---|---|---|
| Scenario | M | F | Allele | (M, p_{A}) | (M, F, p_{A}) |
| 1 | RR | rrr | Maternal | (1 − p_{A})² | 1 − p_{A} |
| 2 | RR | rra | Maternal | (1 − p_{A})² | p_{A} |
| 3 | RR | raa | Maternal | (1 − p_{A})² | 0 |
| 4 | RR | aaa | Maternal | (1 − p_{A})² | 0 |
| 5 | RA | rrr | Maternal | 2p_{A} (1 − p_{A}) | (1 − p_{A})/2 |
| 6 | RA | rra | Maternal | 2p_{A} (1 − p_{A}) | p_{A}/2 |
| 7 | RA | raa | Maternal | 2p_{A} (1 − p_{A}) | (1 − p_{A})/2 |
| 8 | RA | aaa | Maternal | 2p_{A} (1 − p_{A}) | p_{A}/2 |
| 9 | AA | rrr | Maternal | p_{A}² | 0 |
| 10 | AA | rra | Maternal | p_{A}² | 0 |
| 11 | AA | raa | Maternal | p_{A}² | 1 − p_{A} |
| 12 | AA | aaa | Maternal | p_{A}² | p_{A} |
| 13 | RR | rrr | Paternal | (1 − p_{A})² | 1 − p_{A} |
| 14 | RR | rra | Paternal | (1 − p_{A})² | 0 |
| 15 | RR | raa | Paternal | (1 − p_{A})² | p_{A} |
| 16 | RR | aaa | Paternal | (1 − p_{A})² | 0 |
| 17 | RA | rrr | Paternal | 2p_{A} (1 − p_{A}) | (1 − p_{A})/2 |
| 18 | RA | rra | Paternal | 2p_{A} (1 − p_{A}) | (1 − p_{A})/2 |
| 19 | RA | raa | Paternal | 2p_{A} (1 − p_{A}) | p_{A}/2 |
| 20 | RA | aaa | Paternal | 2p_{A} (1 − p_{A}) | p_{A}/2 |
| 21 | AA | rrr | Paternal | p_{A}² | 0 |
| 22 | AA | rra | Paternal | p_{A}² | 1 − p_{A} |
| 23 | AA | raa | Paternal | p_{A}² | 0 |
| 24 | AA | aaa | Paternal | p_{A}² | p_{A} |

The second column lists maternal genotypes M (uppercase; possible values: RR, RA, and AA). Third column: fetal genotypes F affected by duplications (lowercase; possible values: rrr, rra, raa, aaa). Fourth column indicates the parental origin of the deleted allele. Fifth and sixth columns list prior probabilities w_{M} (M, p_{A}) and w_{F} (M, F, p_{A}) of maternal and fetal genotypes per scenario, based on population alternate allele frequency p_{A}

The model operates with predefined (or user-defined) prior probabilities of duplications and deletions, p_{I} and p_{D}, respectively. The prior probability of diploid scenario is obtained as 1−p_{D}−p_{I}.

The next step evaluates likelihood values [symbol] (n_{R},n_{A}|f,M,F) per scenario,

TABLE 0.7
Fetal Duplications: Allele Count Expectations.

| Scenario | M | F | λ_{R} (M, F, f, N) | λ_{A} (M, F, f, N) |
|---|---|---|---|---|
| 1, 13 | RR | rrr | N (1 + f/2) | 0 |
| 2, 14 | RR | rra | N | N f /2 |
| 3, 15 | RR | raa | N (1 − f/2) | N f |
| 4, 16 | RR | aaa | N (1 − f) | 3N f/2 |
| 5, 17 | RA | rrr | N (½ + f) | N (1 − f)/2 |
| 6, 18 | RA | rra | N (1 + f)/2 | N/2 |
| 7, 19 | RA | raa | N/2 | N (1 + f)/2 |
| 8, 20 | RA | aaa | N (1 − f)/2 | N (½ + f) |
| 9, 21 | AA | rrr | 3N f/2 | N (1 − f) |
| 10, 22 | AA | rra | N f | N (1 − f/2) |
| 11, 23 | AA | raa | N f/2 | N |
| 12, 24 | AA | aaa | 0 | N (1 + f/2) |

The first column lists fetal duplication scenarios, as defined in Table 0.6. The second and third columns list maternal and fetal genotypes in upper- and lowercase letters, respectively. Fourth and fifth columns show relationships between fetal fraction f and mathematical expectations λ_{R} = [symbol] (n_{R}) and λ_{A} = [symbol] (n_{A}) for reference and alternate allele counts, respectively. All scenarios where allele counts do not depend on fetal fraction f are uninformative. The symbol N stands for the expected total read count at the given locus. Locus-specific N is obtained from the total observed read count across the SNP panel (sum over all loci), multiplied by a locus-specific factor m.

including all euploid scenarios ( [symbol] . ??), all fetal deletion scenarios (Tbl. 0.4), and all fetal duplication scenarios (Tbl. 0.6). The likelihoods are obtained as products of two Poisson distributions per locus (one for reference counts and one for alternate allele counts). The Poisson expectation values are listed in Tbl. ?? for diploid scenarios, Tbl. 0.5 for fetal deletions, and Tbl. 0.7 for fetal duplications. The expressions adopt the same form as previously used to estimate fetal fraction (cf. Eq. ??). These likelihoods are only evaluated for the loci that are being tested for copy number variations.

The scenario specific likelihoods are next multiplied with prior probabilities of deletions (p_{D}), duplications (p_{I}), or diploid genotypes, as appropriate.

Next, likelihoods are also multiplied with weights [symbol] _{M}(M,p_{A}) and [symbol] _{F} (M,F,p_{A}) listed in tables ??, 0.4, and 0.6. For brevity, future references to likelihoods [symbol] (n_{R},n_{A}|f,M,F) assume that the prior probabilities p_{D}, p_{I} and weights [symbol] _{M}(M,p_{A}), [symbol] _{F}(M,F,p_{A}) are absorbed into the corresponding products.

The likelihoods [symbol] (n_{R},n_{A}|f,M,F) are functions of the fetal fraction f. The next step multiplies these likelihoods with the posterior distribution of fetal fraction values [symbol] (f|{n_{R},n_{A}}) obtained in the first step (not necessarily on the same set of SNP loci as the ones that are tested for copy-number variations). The resulting products are then integrated over the entire fetal fraction range, yielding prior distribution of allele counts [symbol] (n_{R},n_{A}|M,F) marginalized over fetal fractions and only dependent on maternal and fetal genotypes M and F, respectively.

The next step sums up the values [symbol] (n_{R},n_{A}|M,F) over all scenarios M, F, yielding prior count probabilities [symbol] (n_{R},n_{A}).

In the next step, scenario likelihoods [symbol] (n_{R},n_{A}|f,M,F) (including priors p_{D}, p_{I} and weights [symbol] _{M}, [symbol] _{F}) are multiplied by posterior distribution of fetal fractions [symbol] (f|{n_{R},n_{A}}) and divided by the count prior [symbol] (n_{R},n_{A}). Some embodiments omit division by the count prior. The resulting function of fetal fraction f is then integrated over the entire range of f. This integration yields the posterior probabilities of different genotype combinations M, F, one value per scenario.

Finally, the genotype scenario with highest posterior probability is selected as the option with most data support. If the most likely scenario has single fetal allele, the fetus has a deletion at that locus. If the fetal genotype F in the most likely scenario has three alleles, the procedure detected a duplication. Otherwise, the genomic region is diploid both in maternal and fetal genomes.

### J. Single Cell and Circulating Cell-Free Fetal DNA Haplotyping Using Minor Allele Frequencies

Notation

| | |
|---|---|
| 1. | R: Reference allele. |
| 2. | A: Alternate allele. |
| 3. | PH1: Phased paternal allele at a given locus, arbitrarily assigned index one. |
| | PH1 can take values R or A. |
| 4. | PH2: Phased paternal allele at a given locus, assigned index two to distinguish |
| | it from PH2. PH1 can take values R or A. |
| 5. | MH1: Phased maternal allele at a given locus, arbitrarily assigned index one. |
| | MH1 can take values R or A. |
| 6. | MH2: Phased maternal allele at a given locus, assigned index two to distinguish |
| | it from MH1. MH2 can take values R or A. |
| 7. | P: Paternal homologue transmitted to the offspring. Possible values: P ϵ |
| | {None, PH1, PH2, PH1 × 2, PH2 × 2 PH1 + PH2}. The suffix “x2” denotes |
| | duplication of a given homologue. The sign “+” indicates that both paternal |
| | homologues are passed to the offspring. Value None indicates absence of the |
| | paternal homologue in the offspring at the given locus. |
| 8. | M: Maternal homologue transmitted to the offspring. Possible values: M ϵ |
| | {None, MH1, MH2, MH1 × 2, MH2 × 2 MH1 + MH2}. The suffix “x2” |
| | denotes duplication of a given homologue. The sign “+” indicates that both |
| | maternal homologues are passed to the offspring. Value None indicates absence |
| | of the maternal homologue in the offspring at the given locus. |
| 9. | F: Transmission pattern—a combination of phased paternal and maternal |
| | homologues found in the offspring. Possible values in diploid offspring include |
| | PH1.MH1, PH1.MH2, PH2.MH1, and PH2.MH2 (the period being used to |
| | separate paternally and maternally inherited homologues). Deletions, uniparental |
| | disomy scenarios, and duplications involve additional combinations of parental |
| | alleles, such as PH1. None in the case of paternally inherited PH1 and deleted |
| | maternal allele, or PH1 × 2.MH1 in the case of paternal duplication of PH1 |
| | in the presence of maternal allele MH1. |
| 10. | scDNA: Single-cell DNA, also including DNA harvested from a pool of multiple |
| | genetically identical cells. |
| 11. | [symbol] _{R}(F): Multiplier used to evaluate the mathematical expectation for the num- |
| | ber of scDNA reads containing the reference allele and originating from a given |
| | transmission pattern F. |
| 12. | [symbol] _{A}(F): Multiplier used to evaluate the mathematical expectation for the num- |
| | ber of scDNA reads containing the alternate allele and originating from a given |
| | transmission pattern F. |
| 13. | ccfDNA: Circulating cell-free DNA. |
| 14. | ƒ: Fetal fraction: the fractional contribution of fetal DNA in circulating cell-free |
| | DNA extracted from maternal plasma. |
| 15. | θ_{R}(F, ƒ): Multiplier used to evaluate the mathematical expectation for the |
| | number of ccfDNA reads containing the reference allele and originating from a |
| | given transmission pattern F. [symbol] _{R}(F, ƒ) is a function of the transmission pattern |
| | F and the fetal fraction ƒ. |
| 16. | θ_{R}(F, ƒ): Multiplier used to evaluate the mathematical expectation for the |
| | number of ccfDNA reads containing the alternate allele and originating from a |
| | given transmission pattern F. [symbol] _{A}(F, ƒ) is a function of the transmission pattern |
| | F and the fetal fraction ƒ. |
| 17. | n_{R}: Number of reads aligned to the given locus and containing the reference |
| | allele R. |
| 18. | n_{A}: Number of reads aligned to the given locus and containing the alternate |
| | allele A |
| 19. | N: Total expected number of reads aligned to a given locus. This is the expec- |
| | tation value, not the actually observed total read count n_{R} + n_{A}. |
| 20. | λ_{R}(N, F): Expected number of scDNA reference allele counts at a given locus, |
| | given the expected total read count N and the transmission pattern F. |
| 21. | λ_{A}(N, F): Expected number of scDNA alternate allele counts at a given locus, |
| | given the expected total read count N and the transmission pattern F. |
| 22. | μ_{R}(N, F, ƒ): Expected number of ccfDNA reference allele counts at a given |
| | locus, given the expected total read count N, the transmission pattern F, and |
| | the fetal fraction ƒ. |
| 23. | μ_{A}(N, F, ƒ): Expected number of ccfDNA alternate allele counts at a given |
| | locus, given the expected total read count N, the transmission pattern F, and |
| | the fetal fraction ƒ. |
| 24. | Φ: Set of all values F included in the model. |
| 25. | [symbol] (n_{R}, n_{A}\|F, P, F): Joint conditional probability of observing allele frequencies |
| | n_{R} and n_{A} at a given locus in a scDNA or ccfDNA experiment, given the trans- |
| | mission pattern F and a certain set of maternal and paternal allele realizations |
| | P and M. |
| 26. | Z: Normalization constant for [symbol] (n_{R}, n_{A}\|F, P, F). |
| 27. | Ξ: Normalization constant for [symbol] (n_{R}, n_{A}\|F, ƒ). |
| 28. | [symbol] (F): Likelihood for a region covering one or more SNP loci. |
| 29. | F₀: The most likely haplotype transmission pattern for a given region. |

Background

In addition to whole chromosome aneuploidies and subchromosomal copy-number variation, single nucleotide polymorphisms can also predispose the offspring to inherited diseases. In particular, there are more than 10,000 known monogenic diseases, with the incidence at birth estimated to approach 1%. While medically important, prenatal detection of fetal monogenic diseases from ccfDNA is hampered by the overwhelming background of maternal DNA. In particular, targeting multiple disease loci proves prohibitively expensive and difficult to scale for routine testing and/or screening purposes. A particularly promising approach takes advantage of parental carrier and phasing information, when available. Rather than targeting disease loci, the phasing approach targets a well-defined set of SNP loci with significant alternate allele frequency in the population. For example, Agilent's SureSelect Target Enrichment platform for Illumina Paired-End sequencing allows targeting ≈250,000 SNPs, with average distance between two neighboring SNPs on the order of 12.4 kb. At minor allele frequency close to 0.5, the number of paternally informative loci in a typical human genome exceeds 62,000. The average distance between two neighboring paterally informative loci is therefore close to 50 kb. With a similar number of maternally informative loci, the resolution of identifying disease-carrying haplotypes is close to 25 kb. If on average a typical human chromosome contains 1.6 recombination events J. Wang, et al., Cell 150:402, 2012, Milo Phillips (2016): Cell biology by the numbers, Garland Science, Taylor & Francis Group, LLC, New York), this resolution is sufficient to reliably detect transmission of parental monogenic diseases to the offspring without actually targeting the disease loci, thus greatly simplifying the workflow and reducing its cost. Measuring allele counts at well characterized, not necessarily disease-related, SNP loci flanking the monogenic disease hotspots is sufficient to ascertain whether the offspring received the variant homologue from the carrier parent.

The current disclosure proposes a novel approach to establishing haplotype blocks inherited paternally and/or maternally when parental genomes are phased and offspring's allele frequencies are measured. Offspring allele frequencies can be quantified either from pure prenatal or neonatal sample (for example, single cell DNA from non-invasively harvested trophoblasts, obtained through cervical swab, Moser G, Drewlo S, Huppertz B, Armant DR (2018): Trophoblast retrieval and isolation from the cervix: origins of cervical trophoblasts and their potential value for risk assessment of ongoing pregnancies. Hum Reprod Update. 2018 Jul. 1; 24(4):484-496. doi: 10.1093/humupd/dmy008) or from a mixture of maternal and fetal DNA (as in ccfDNA). If parental carrier information is available, this novel approach enables accurate determination of whether the disease-carrying parental haplotype block(s) have been transmitted to the offspring. The same protocol is also applicable to in-vitro fertilization (IVF) quality control, based on sequencing of single cells biopsied from blastocysts (Kuznyetsov et al. (2018): Evaluation of a novel non-invasive preimplantation genetic screening approach. PLoS One. 2018; 13(5): e0197262. Published online 2018 May 10. doi: 10.1371/journal.pone.0197262).

Overview of the Proposed Algorithm

The proposed approach to haplotyping the offspring assumes that a plurality of genomic loci, such as biallelic single nucleotide polymorphisms (SNPs), can be targeted and their allele frequencies can be measured. These loci constitute an SNP panel. The approach also assumes that parental genotypes are known across the entire SNP panel. Further, the approach assumes that parental alleles have been phased and that haplotype block structure is available at least for the parent who is the disease carrier. In some embodiments, only one parent's haplotypes are known. In some embodiments, both parents' haplotypes are known. Finally, the approach assumes that offspring's allele frequencies can be quantified either by NGS or by microarray measurements, such as aCGH.

In summary, the proposed approach haplotypes the offspring by evaluating posterior probabilities [symbol] (F|n_{R},n_{A},P,M) of parental haplotype transmission patterns F, given the measured fetal allele frequencies n_{R}, n_{A} and the known parental haplotypes P, M. The transmission patterns F represent a combination of offspring's genotypes and their possible parental origins. For example, in a diploid fetus (excluding uniparetal disomy), the transmission pattern F can take four possible values: PH1.MH1, PH1.MH2, PH2.MH1, or PH2.MH2, where PHn and MHn are phased paternal and maternal alleles, respectively. Each transmitted parental allele can take values R or A (reference and alternate allele), depending on haplotypes found in the parents. The posterior probabilities [symbol] (F|n_{R},n_{A},P,M) are evaluated per locus across the entire SNP panel. Next, a sequence of per-locus posterior probabilities corresponding to adjacent genomic loci is segmented into homogeneous fetal regions representing inherited haplotype blocks. Segmentation edges represent recombination sites. The haplotyping procedure infers transmission of disease based on whether the affected haplotype is passed from the carrier parent to the offspring. The procedure is also capable of detecting whole chromosome aneuploidies, subchromosomal copy number variations, as well as parental origin of the duplicated or missing fetal genomic segments. Combined with the procedures to measure fetal fraction (described in Section 206), this approach offers non-invasive prenatal detection of the full spectrum of monogenic diseases as well as copy number variations in a single sequencing run.

Transmission Patterns

The fetal transmission pattern F is a categorical variable that can take a value from a countably infinite discrete set. The number of practically relevant scenarios is small. In the case of the heterozygous deletion of the maternally inherited allele, the possible F values include PH1.None and PH2.None. If, in addition to the loss of the maternal allele, the paternally inherited haplotype is duplicated, the variable F takes the value PH1×2.None or PH2×2.None. When both paternal alleles are present, but the maternal allele is missing, F is assigned the value PH1+PH2.None. Similarly, missing paternal allele allows the following F values: None, MH1, None, MH2, None, MH1×2, None, MH2×2, or None, MH1+MH2. Higher paternal copy numbers combined with missing maternal alleles, as well as the symmetrically configured scenarios, can also be modeled in the same fashion.

A euploid locus inherits one allele from the father and one from the mother, allowing the following four values for F: PH1.MH1, PH1.MH2, PH2.MH1, and PH2.MH2.

As the copy number increases, the number of possible transmission patterns F grows as well. For example, a subset of possible duplications of paternally inherited allele(s) include PH1×2.MH1, PH1×2.MH2, PH2×2.MH1, PH2×2.MH2, PH1+PH2.MH1, PH1+PH2.MH2. Symmetrical configurations PH1.MH1×2, PH1.MH2×2, PH2.MH1×2, PH2.MH2×2, PH1.MH1+MH2, and PH2.MH1+MH2 account for maternal duplications. Additional scenarios are presumably less likely as their complexity increases, but the model can include them when appropriate.

Poisson Expectations

In a biallelic SNP, any one of the four parental alleles (PH1, PH2, MH1, and MH2) can either match the reference allele R or the alternate allele A, leading to a number of combinatorial possibilities for allelic realization of the transmission pattern F. These possible realizations then determine the possible expectation values for measured allele counts n_{R} and n_{A} in a single-cell experiment or in a circulating cell-free DNA experiment. The expectations λ_{R} and λ_{A} in the prenatal single-cell measurements (such as those obtained on DNA from trophoblasts non-invasively obtained by cervical swab) are proportional to the total expected read count N at the given locus and to the multiplier [symbol] _{R}(F) (for reference allele) or [symbol] _{A}(F) (for alternate allele):

λ_{R}(F,N)= [symbol] _{R}(F)N (56)

λ_{A}(F,N)= [symbol] _{A}(F)N (57)

The multipliers [symbol] _{R}(F) and [symbol] _{A}(F) absorb the dependence of λ_{R}, λ_{A} on the fetal transmission pattern F. The model can also include additional factors as either multiplicative or additive modifiers, such as mappability bias m, GC bias G(g,L,Q,g₀), drop-out rate, drop-out allele rate, and drop-in allele rate. N is a product of the total number of reads across all loci in the SNP panel, a factor representing a fraction of all reads expected to map to the locus in question, and a factor combining the above mentioned biases.

In circulating cell-free DNA measurements, the expected allele frequencies μ_{R} and μ_{A} must also account for the fetal fraction f. The proportionality factors θ_{R}(F,f) and θ_{A}(F,f) reflect the relative amount of fetal DNA in the sample, as well as the allelic realization of the fetal transmission pattern F.

μ_{R}(F,f,N)=θ_{R}(F,f)N (58)

μ_{A}(F,f,N)=θ_{A}(F,f)N (59)

Tables of Multiplier Values

Tables 0.8-0.19 list a subset of possible parental allele combinations, fetal inheritance patterns, scDNA multipliers [symbol] _{R} and [symbol] _{A}, and ccfDNA multipliers θ_{R} and θ_{A}. In the interest of space, these tables cover some of the more frequently encountered scenarios, while omitting the less likely ones, but can be straightforwardly extended to include additional possibilities.

Both scDNA multipliers [symbol] _{R}, [symbol] _{A} and ccfDNA multipliers θ_{R}, θ_{A} depend on actual allele assignments P and M to parental haplotypes PH1, PH2, MH1, and MH2 (or parental allele realizations). These dependencies are tabulated Tables 0.8-0.19. However, to simplify equations and save space, the notation does not stress this dependence. For example, rather than explicitly writing out [symbol] _{R}(F,P,M), the tables and equations below simply refer to [symbol] _{R}(F).

TABLE 0.8
Fetal Monosomy: Paternal Allele Transmission Scenarios

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 1 | R | R | R | R | PH1.None | 1/2 | 0 | 1 − f/2 | 0 |
| 2 | R | R | R | R | PH2.None | 1/2 | 0 | 1 − f/2 | 0 |
| 3 | R | R | R | A | PH1.None | 1/2 | 0 | 1/2 | (1 − f)/2 |
| 4 | R | R | R | A | PH2.None | 1/2 | 0 | 1/2 | (1 − f)/2 |
| 5 | R | R | A | R | PH1.None | 1/2 | 0 | 1/2 | (1 − f)/2 |
| 6 | R | R | A | R | PH2.None | 1/2 | 0 | 1/2 | (1 − f)/2 |
| 7 | R | R | A | A | PH1.None | 1/2 | 0 | f/2 | 1 − f |
| 8 | R | R | A | A | PH2.None | 1/2 | 0 | f/2 | 1 − f |
| 9 | R | A | R | R | PH1.None | 1/2 | 0 | 1 − f/2 | 0 |
| 10 | R | A | R | R | PH2.None | 0 | 1/2 | 1 − f | f/2 |
| 11 | R | A | R | A | PH1.None | 1/2 | 0 | 1/2 | (1 − f)/2 |
| 12 | R | A | R | A | PH2.None | 0 | 1/2 | (1 − f)/2 | 1/2 |
| 13 | R | A | A | R | PH1.None | 1/2 | 0 | 1/2 | (1 − f)/2 |
| 14 | R | A | A | R | PH2.None | 0 | 1/2 | (1 − f)/2 | 1/2 |
| 15 | R | A | A | A | PH1.None | 1/2 | 0 | f/2 | 1 − f |
| 16 | R | A | A | A | PH2.None | 0 | 1/2 | 0 | 1 − f/2 |
| 17 | A | R | R | R | PH1.None | 0 | 1/2 | 1 − f | f/2 |
| 18 | A | R | R | R | PH2.None | 1/2 | 0 | 1 − f/2 | 0 |
| 19 | A | R | R | A | PH1.None | 0 | 1/2 | (1 − f)/2 | 1/2 |
| 20 | A | R | R | A | PH2.None | 1/2 | 0 | 1/2 | (1 − f)/2 |
| 21 | A | R | A | R | PH1.None | 0 | 1/2 | (1 − f)/2 | 1/2 |
| 22 | A | R | A | R | PH2.None | 1/2 | 0 | 1/2 | (1 − f)/2 |
| 23 | A | R | A | A | PH1.None | 0 | 1/2 | 0 | 1 − f/2 |
| 24 | A | R | A | A | PH2.None | 1/2 | 0 | f/2 | 1 − f |
| 25 | A | A | R | R | PH1.None | 0 | 1/2 | 1 − f | f/2 |
| 26 | A | A | R | R | PH2.None | 0 | 1/2 | 1 − f | f/2 |
| 27 | A | A | R | A | PH1.None | 0 | 1/2 | (1 − f)/2 | 1/2 |
| 28 | A | A | R | A | PH2.None | 0 | 1/2 | (1 − f)/2 | 1/2 |
| 29 | A | A | A | R | PH1.None | 0 | 1/2 | (1 − f)/2 | 1/2 |
| 30 | A | A | A | R | PH2.None | 0 | 1/2 | (1 − f)/2 | 1/2 |
| 31 | A | A | A | A | PH1.None | 0 | 1/2 | 0 | 1 − f/2 |
| 32 | A | A | A | A | PH2.None | 0 | 1/2 | 0 | 1 − f/2 |

Likelihoods

In single-cell experiments, the joint conditional probability of observing allele counts n_{R} and n_{A} given fetal transmission pattern F is modeled using a product

TABLE 0.9
Fetal Uniparental Disomy: Paternal Allele Transmission Scenarios

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 1 | R | R | R | R | PH1x2.None | 1 | 0 | 1 | 0 |
| 2 | R | R | R | R | PH2x2.None | 1 | 0 | 1 | 0 |
| 3 | R | R | R | R | PH1 + PH2.None | 1 | 0 | 1 | 0 |
| 4 | R | R | R | A | PH1x2.None | 1 | 0 | (1 + f)/2 | (1 − f)/2 |
| 5 | R | R | R | A | PH2x2.None | 1 | 0 | (1 + f)/2 | (1 − f)/2 |
| 6 | R | R | R | A | PH1 + PH2.None | 1 | 0 | (1 + f)/2 | (1 − f)/2 |
| 7 | R | R | A | R | PH1x2.None | 1 | 0 | (1 + f)/2 | (1 − f)/2 |
| 8 | R | R | A | R | PH2x2.None | 1 | 0 | (1 + f)/2 | (1 − f)/2 |
| 9 | R | R | A | R | PH1 + PH2.None | 1 | 0 | (1 + f)/2 | (1 − f)/2 |
| 10 | R | R | A | A | PH1x2.None | 1 | 0 | f | 1 − f |
| 11 | R | R | A | A | PH2x2.None | 1 | 0 | f | 1 − f |
| 12 | R | R | A | A | PH1 + PH2.None | 1 | 0 | f | 1 − f |
| 13 | R | A | R | R | PH1x2.None | 1 | 0 | 1 | 0 |
| 14 | R | A | R | R | PH2x2.None | 0 | 1 | 1 − f | f |
| 15 | R | A | R | R | PH1 + PH2.None | 1/2 | 1/2 | 1 − f/2 | f/2 |
| 16 | R | A | R | A | PH1x2.None | 1 | 0 | (1 + f)/2 | (1 − f)/2 |
| 17 | R | A | R | A | PH2x2.None | 0 | 1 | (1 − f)/2 | (1 + f)/2 |
| 18 | R | A | R | A | PH1 + PH2.None | 1/2 | 1/2 | 1/2 | 1/2 |
| 19 | R | A | A | R | PH1x2.None | 1 | 0 | (1 + f)/2 | (1 − f)/2 |
| 20 | R | A | A | R | PH2x2.None | 0 | 1 | (1 − f)/2 | (1 + f)/2 |
| 21 | R | A | A | R | PH1 + PH2.None | 1/2 | 1/2 | 1/2 | 1/2 |
| 22 | R | A | A | A | PH1x2.None | 1 | 0 | f | 1 − f |
| 23 | R | A | A | A | PH2x2.None | 0 | 1 | 0 | 1 |
| 24 | R | A | A | A | PH1 + PH2.None | 1/2 | 1/2 | f/2 | (1 − f)/2 |

of two Poisson distributions, assuming independence of data generated by the two alleles:

$$ℙ ( {n}_{R} , {n}_{A} \vert F , P , M ) = \frac{1}{Z} 𝒫 ( {n}_{R} , {λ}_{R} ( F , N ) ) 𝒫 ( {n}_{A} , {λ}_{A} ( F , N ) ) = \frac{1}{Z} 𝒫 ( {n}_{R} , {λ}_{R} ( F , N ) ) 𝒫 ( {n}_{A} , {λ}_{A} ( F , N ) ) \qquad ( 60 )$$

The joint conditional probability [symbol] (n_{R},n_{A}|F,P,F) is also referred to as likelihood. P and M are particular allele assignments to transmitted parental alleles PH1, PH2, MH1, and MH2. The mathematical expectations λ_{R}(F) and λ_{A}(F) are

TABLE 0.10
Fetal Uniparental Disomy: Paternal Allele
Transmission Scenarios (Continued)

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 25 | A | R | R | R | PH1x2.None | 0 | 1 | 1 − f | f |
| 26 | A | R | R | R | PH2x2.None | 1 | 0 | 1 | 0 |
| 27 | A | R | R | R | PH1 + PH2.None | 1 | 0 | 1 − f/2 | f/2 |
| 28 | A | R | R | A | PH1x2.None | 0 | 1 | (1 − f)/2 | (1 + f)/2 |
| 29 | A | R | R | A | PH2x2.None | 1 | 0 | (1 + f)/2 | (1 − f)/2 |
| 30 | A | R | R | A | PH1 + PH2.None | 1/2 | 1/2 | 1/2 | 1/2 |
| 31 | A | R | A | R | PH1x2.None | 0 | 1 | (1 − f)/2 | (1 + f)/2 |
| 32 | A | R | A | R | PH2x2.None | 1 | 0 | (1 + f)/2 | (1 − f)/2 |
| 33 | A | R | A | R | PH1 + PH2.None | 1/2 | 1/2 | 1/2 | 1/2 |
| 34 | A | R | A | A | PH1x2.None | 0 | 1 | 0 | 1 |
| 35 | A | R | A | A | PH2x2.None | 1 | 0 | f | 1 − f |
| 36 | A | R | A | A | PH1 + PH2.None | 1/2 | 1/2 | f/2 | (1 − f)/2 |
| 37 | A | A | R | R | PH1x2.None | 0 | 1 | 1 − f | f |
| 38 | A | A | R | R | PH2x2.None | 0 | 1 | 1 − f | f |
| 39 | A | A | R | R | PH1 + PH2.None | 0 | 1 | 1 − f | f |
| 40 | A | A | R | A | PH1x2.None | 0 | 1 | (1 − f)/2 | (1 + f)/2 |
| 41 | A | A | R | A | PH2x2.None | 0 | 1 | (1 − f)/2 | (1 + f)/2 |
| 42 | A | A | R | A | PH1 + PH2.None | 0 | 1 | (1 − f)/2 | (1 + f)/2 |
| 43 | A | A | A | R | PH1x2.None | 0 | 1 | (1 − f)/2 | (1 + f)/2 |
| 44 | A | A | A | R | PH2x2.None | 0 | 1 | (1 − f)/2 | (1 + f)/2 |
| 45 | A | A | A | R | PH1 + PH2.None | 0 | 1 | (1 − f)/2 | (1 + f)/2 |
| 46 | A | A | A | A | PH1x2.None | 0 | 1 | 0 | 1 |
| 47 | A | A | A | A | PH2x2.None | 0 | 1 | 0 | 1 |
| 48 | A | A | A | A | PH1 + PH2.None | 0 | 1 | 0 | 1 |

given by Eqs. 56-57. The multipliers [symbol] _{R}(F) and [symbol] _{A}(F) are listed in Tables 0.8-0.19, columns 7-8. The normalization constant Z is obtained by summation over the set Φ of all values F included in the model, with fixed paternal and maternal allele realizations P and M:

$$Z = {\sum }_{F ∈ Φ} 𝒫 ( {n}_{R} , {τ}_{R} ( F ) N ) 𝒫 ( {n}_{A} , {τ}_{A} ( F ) N ) \qquad ( 61 )$$

In a ccfDNA experiment, the likelihood [symbol] (n_{R},n_{A}|F,P,M) is evaluated differently to take into account the fetal fraction f. The scenario corresponding to fetal

TABLE 0.11
Fetal Monosomy: Maternal Allele Transmission Scenarios

| # | PH1 | PH2 | MH1 | MH2 | F | τ(F) | τ_{A}(F) | θ_{R}(F,f) | θ_{A}(F,f) |
|---|---|---|---|---|---|---|---|---|---|
| 1 | R | R | R | R | None, | 1/2 | 0 | 1 − f/2 | 0 |
| | | | | | MH1 | | | | |
| 2 | R | R | R | R | None, | 1/2 | 0 | 1 − f/2 | 0 |
| | | | | | MH2 | | | | |
| 3 | R | R | R | A | None, | 1/2 | 0 | 1/2 | (1 − f)/2 |
| | | | | | MH1 | | | | |
| 4 | R | R | R | A | None, | 0 | 1/2 | (1 − f)/2 | 1/2 |
| | | | | | MH2 | | | | |
| 5 | R | R | A | R | None, | 0 | 1/2 | (1 − f)/2 | 1/2 |
| | | | | | MH1 | | | | |
| 6 | R | R | A | R | None, | 1/2 | 0 | 1/2 | (1 − f)/2 |
| | | | | | MH2 | | | | |
| 7 | R | R | A | A | None, | 0 | 1/2 | 0 | 1 − f/2 |
| | | | | | MH1 | | | | |
| 8 | R | R | A | A | None, | 0 | 1/2 | 0 | 1 − f/2 |
| | | | | | MH2 | | | | |
| 9 | R | A | R | R | None, | 1/2 | 0 | 1 − f/2 | 0 |
| | | | | | MH1 | | | | |
| 10 | R | A | R | R | None, | 1/2 | 0 | 1 − f/2 | 0 |
| | | | | | MH2 | | | | |
| 11 | R | A | R | A | None, | 1/2 | 0 | 1/2 | (1 − f)/2 |
| | | | | | MH1 | | | | |
| 12 | R | A | R | A | None, | 0 | 1/2 | (1 − f)/2 | 1/2 |
| | | | | | MH2 | | | | |
| 13 | R | A | A | R | None, | 0 | 1/2 | (1 − f)/2 | 1/2 |
| | | | | | MH1 | | | | |
| 14 | R | A | A | R | None, | 1/2 | 0 | 1/2 | (1 − f)/2 |
| | | | | | MH2 | | | | |
| 15 | R | A | A | A | None, | 0 | 1/2 | 0 | 1 − f/2 |
| | | | | | MH1 | | | | |
| 16 | R | A | A | A | None, | 0 | 1/2 | 0 | 1 − f/2 |
| | | | | | MH2 | | | | |
| 17 | A | R | R | R | None, | 1/2 | 0 | 1 − f/2 | 0 |
| | | | | | MH1 | | | | |
| 18 | A | R | R | R | None, | 1/2 | 0 | 1 − f/2 | 0 |
| | | | | | MH2 | | | | |
| 19 | A | R | R | A | None, | 1/2 | 0 | 1/2 | (1 − f)/2 |
| | | | | | MH1 | | | | |
| 20 | A | R | R | A | None, | 0 | 1/2 | (1 − f)/2 | 1/2 |
| | | | | | MH2 | | | | |
| 21 | A | R | A | R | None, | 0 | 1/2 | (1 − f)/2 | 1/2 |
| | | | | | MH1 | | | | |
| 22 | A | R | A | R | None, | 1/2 | 0 | 1/2 | (1 − f)/2 |
| | | | | | MH2 | | | | |
| 23 | A | R | A | A | None, | 0 | 1/2 | 0 | 1 − f/2 |
| | | | | | MH1 | | | | |
| 24 | A | R | A | A | None, | 0 | 1/2 | 0 | 1 − f/2 |
| | | | | | MH2 | | | | |
| 25 | A | A | R | R | None, | 1/2 | 0 | 1 − f/2 | 0 |
| | | | | | MH1 | | | | |
| 26 | A | A | R | R | None, | 1/2 | 0 | 1 − f/2 | 0 |
| | | | | | MH2 | | | | |
| 27 | A | A | R | A | None, | 1/2 | 0 | 1/2 | (1 − f)/2 |
| | | | | | MH1 | | | | |
| 28 | A | A | R | A | None, | 0 | 1/2 | (1 − f)/2 | 1/2 |
| | | | | | MH2 | | | | |
| 29 | A | A | A | R | None, | 0 | 1/2 | (1 − f)/2 | 1/2 |
| | | | | | MH1 | | | | |
| 30 | A | A | A | R | None, | 1/2 | 0 | 1/2 | (1 − f)/2 |
| | | | | | MH2 | | | | |
| 31 | A | A | A | A | None, | 0 | 1/2 | 0 | 1 − f/2 |
| | | | | | MH1 | | | | |
| 32 | A | A | A | A | None, | 0 | 1/2 | 0 | 1 − f/2 |
| | | | | | MH2 | | | | |

transmission pattern F is modeled using a product of three terms: the distribution of fetal fractions and two Poisson probabilities with expectations μ_{R}(F,f,N) and μ_{A}(F,f,N) given by Eqs. 58-59. Finally, the three-term product is marginalized

TABLE 0.12
Fetal Uniparental Disomy: Maternal Allele Transmission Scenarios

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 1 | R | R | R | R | None, MH1x2 | 1 | 0 | 1 | 0 |
| 2 | R | R | R | R | None, MH2x2 | 1 | 0 | 1 | 0 |
| 3 | R | R | R | R | None, MH1 + MH2 | 1 | 0 | 1 | 0 |
| 4 | R | R | R | A | None, MH1x2 | 1 | 0 | (1 + f)/2 | (1 − f)/2 |
| 5 | R | R | R | A | None, MH2x2 | 0 | 1 | (1 − f)/2 | (1 + f)/2 |
| 6 | R | R | R | A | None, MH1 + MH2 | 1/2 | 1/2 | 1/2 | 1/2 |
| 7 | R | R | A | R | None, MH1x2 | 0 | 1 | (1 − f)/2 | (1 + f)/2 |
| 8 | R | R | A | R | None, MH2x2 | 1 | 0 | (1 + f)/2 | (1 − f)/2 |
| 9 | R | R | A | R | None, MH1 + MH2 | 1/2 | 1/2 | 1/2 | 1/2 |
| 10 | R | R | A | A | None, MH1x2 | 0 | 1 | 0 | 1 |
| 11 | R | R | A | A | None, MH2x2 | 0 | 1 | 0 | 1 |
| 12 | R | R | A | A | None, MH1 + MH2 | 0 | 1 | 0 | 1 |
| 13 | R | A | R | R | None, MH1x2 | 1 | 0 | 1 | 0 |
| 14 | R | A | R | R | None, MH2x2 | 1 | 0 | 1 | 0 |
| 15 | R | A | R | R | None, MH1 + MH2 | 1 | 0 | 1 | 0 |
| 16 | R | A | R | A | None, MH1x2 | 1 | 0 | (1 + f)/2 | (1 − f)/2 |
| 17 | R | A | R | A | None, MH2x2 | 0 | 1 | (1 − f)/2 | (1 + f)/2 |
| 18 | R | A | R | A | None, MH1 + MH2 | 1/2 | 1/2 | 1/2 | 1/2 |
| 19 | R | A | A | R | None, MH1x2 | 0 | 1 | (1 − f)/2 | (1 + f)/2 |
| 20 | R | A | A | R | None, MH2x2 | 1 | 0 | (1 + f)/2 | (1 − f)/2 |
| 21 | R | A | A | R | None, MH1 + MH2 | 1/2 | 1/2 | 1/2 | 1/2 |
| 22 | R | A | A | A | None, MH1x2 | 0 | 1 | 0 | 1 |
| 23 | R | A | A | A | None, MH2x2 | 0 | 1 | 0 | 1 |
| 24 | R | A | A | A | None, MH1 + MH2 | 0 | 1 | 0 | 1 |

over all possible fetal fraction values to factor out f, giving the following expression:

$$ℙ ( {n}_{R} , {n}_{A} \vert F , P , M ) = \frac{1}{Ξ} {\int }_{0}^{1} df ℙ ( f \vert \{ {n}_{R} , {n}_{A} \} ) 𝒫 ( {n}_{R} , {μ}_{R} ( F , f , N ) ) 𝒫 ( {n}_{A} , {μ}_{A} ( F , f , N ) ) = \frac{1}{Ξ} {\int }_{0}^{1} df ℙ ( f \vert \{ {n}_{R} , {n}_{A} \} ) 𝒫 ( {n}_{R} , {θ}_{R} ( F , f ) N ) 𝒫 ( {n}_{A} , {θ}_{A} ( F , f ) N ) \qquad ( 62 )$$

The multipliers θ_{R}(F,f) and θ_{A}(F,f) are listed in Tables 0.8-0.19, columns 9-10. Eq. 62 assumes that the processes of generating n_{R} and n_{A} are independent.

TABLE 0.13
Fetal Uniparental Disomy: Maternal Allele Transmission Scenarios (Continued)

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 25 | A | R | R | R | None, MH1x2 | 1 | 0 | 1 | 0 |
| 26 | A | R | R | R | None, MH2x2 | 1 | 0 | 1 | 0 |
| 27 | A | R | R | R | None, MH1 + MH2 | 1 | 0 | 1 | 0 |
| 28 | A | R | R | A | None, MH1x2 | 1 | 0 | (1 + f)/2 | (1 − f)/2 |
| 29 | A | R | R | A | None, MH2x2 | 0 | 1 | (1 − f)/2 | (1 + f)/2 |
| 30 | A | R | R | A | None, MH1 + MH2 | 1/2 | 1/2 | 1/2 | 1/2 |
| 31 | A | R | A | R | None, MH1x2 | 0 | 1 | (1 − f)/2 | (1 + f)/2 |
| 32 | A | R | A | R | None, MH2x2 | 1 | 0 | (1 + f)/2 | (1 − f)/2 |
| 33 | A | R | A | R | None, MH1 + MH2 | 1/2 | 1/2 | 1/2 | 1/2 |
| 34 | A | R | A | A | None, MH1x2 | 0 | 1 | 0 | 1 |
| 35 | A | R | A | A | None, MH2x2 | 0 | 1 | 0 | 1 |
| 36 | A | R | A | A | None, MH1 + MH2 | 0 | 1 | 0 | 1 |
| 37 | A | A | R | R | None, MH1x2 | 1 | 0 | 1 | 0 |
| 38 | A | A | R | R | None, MH2x2 | 1 | 0 | 1 | 0 |
| 39 | A | A | R | R | None, MH1 + MH2 | 1 | 0 | 1 | 0 |
| 40 | A | A | R | A | None, MH1x2 | 1 | 0 | (1 + f)/2 | (1 − f)/2 |
| 41 | A | A | R | A | None, MH2x2 | 0 | 1 | (1 − f)/2 | (1 + f)/2 |
| 42 | A | A | R | A | None, MH1 + MH2 | 1/2 | 1/2 | 1/2 | 1/2 |
| 43 | A | A | A | R | None, MH1x2 | 0 | 1 | (1 ? f)/2 | (1 + f)/2 |
| 44 | A | A | A | R | None, MH2x2 | 1 | 0 | (1 − f)/2 | (1 − f)/2 |
| 45 | A | A | A | R | None, MH1 + MH2 | 1/2 | 1/2 | 1/2 | 1/2 |
| 46 | A | A | A | A | None, MH1x2 | 0 | 1 | 0 | 1 |
| 47 | A | A | A | A | None, MH2x2 | 0 | 1 | 0 | 1 |
| 48 | A | A | A | A | None, MH1 + MH2 | 0 | 1 | 0 | 1 |

The normalization constant Ξ is obtained by summation over all values of F included in the model:

$$Ξ = {\sum }_{F ∈ Φ} df ℙ ( f \vert \{ {n}_{R} , {n}_{A} \} ) 𝒫 ( {n}_{R} , {θ}_{R} ( F , f ) N ) 𝒫 ( {n}_{A} , {θ}_{A} ( F , f ) N ) \qquad ( 63 )$$

The posterior distribution [symbol] (f|{n_{R},n_{A}}) featuring in Eqs. 62-63 can be evaluated as described in Section 206. In some embodiments, fetal fraction prior [symbol] (f) is used instead of [symbol] (f|{n_{R},n_{A}}) (see Section 206). In some embodiments, a single (most likely) point estimate of f is used, rather than integrating the distribution of

TABLE 0.14
Euploid Fetus: Parental Allele Transmission

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 1 | R | R | R | R | PH1.MH1 | 1 | 0 | 1 | 0 |
| 2 | R | R | R | R | PH1.MH2 | 1 | 0 | 1 | 0 |
| 3 | R | R | R | R | PH2.MH1 | 1 | 0 | 1 | 0 |
| 4 | R | R | R | R | PH2.MH2 | 1 | 0 | 1 | 0 |
| 5 | R | R | R | A | PH1.MH1 | 1 | 0 | (1 + f)/2 | (1 − f)/2 |
| 6 | R | R | R | A | PH1.MH2 | 1/2 | 1/2 | 1/2 | 1/2 |
| 7 | R | R | R | A | PH2.MH1 | 1 | 0 | (1 + f)/2 | (1 − f)/2 |
| 8 | R | R | R | A | PH2.MH2 | 1/2 | 1/2 | 1/2 | 1/2 |
| 9 | R | R | A | R | PH1.MH1 | 1/2 | 1/2 | 1/2 | 1/2 |
| 10 | R | R | A | R | PH1.MH2 | 1 | 0 | (1 + f)/2 | (1 − f)/2 |
| 11 | R | R | A | R | PH2.MH1 | 1/2 | 1/2 | 1/2 | 1/2 |
| 12 | R | R | A | R | PH2.MH2 | 1 | 0 | (1 + f)/2 | (1 − f)/2 |
| 13 | R | R | A | A | PH1.MH1 | 1/2 | 1/2 | f/2 | 1 − f/2 |
| 14 | R | R | A | A | PH1.MH2 | 1/2 | 1/2 | f/2 | 1 − f/2 |
| 15 | R | R | A | A | PH2.MH1 | 1/2 | 1/2 | f/2 | 1 − f/2 |
| 16 | R | R | A | A | PH2.MH2 | 1/2 | 1/2 | f/2 | 1 − f/2 |
| 17 | R | A | R | R | PH1.MH1 | 1 | 0 | 1 | 0 |
| 18 | R | A | R | R | PH1.MH2 | 1 | 0 | 1 | 0 |
| 19 | R | A | R | R | PH2.MH1 | 1/2 | 1/2 | f/2 | 1 − f/2 |
| 20 | R | A | R | R | PH2.MH2 | 1/2 | 1/2 | f/2 | 1 − f/2 |
| 21 | R | A | R | A | PH1.MH1 | 1 | 0 | (1 + f)/2 | (1 − f)/2 |
| 22 | R | A | R | A | PH1.MH2 | 1/2 | 1/2 | 1/2 | 1/2 |
| 23 | R | A | R | A | PH2.MH1 | 1/2 | 1/2 | 1/2 | 1/2 |
| 24 | R | A | R | A | PH2.MH2 | 0 | 1 | (1 − f)/2 | (1 + f)/2 |
| 25 | R | A | A | R | PH1.MH1 | 1/2 | 1/2 | 1/2 | 1/2 |
| 26 | R | A | A | R | PH1.MH2 | 1 | 0 | (1 + f)/2 | (1 − f)/2 |
| 27 | R | A | A | R | PH2.MH1 | 0 | 1 | (1 − f)/2 | (1 + f)/2 |
| 28 | R | A | A | R | PH2.MH2 | 1/2 | 1/2 | 1/2 | 1/2 |
| 29 | R | A | A | A | PH1.MH1 | 1/2 | 1/2 | f/2 | 1 − f/2 |
| 30 | R | A | A | A | PH1.MH2 | 1/2 | 1/2 | f/2 | 1 − f/2 |
| 31 | R | A | A | A | PH2.MH1 | 0 | 1 | 0 | 1 |
| 32 | R | A | A | A | PH2.MH2 | 0 | 1 | 0 | 1 |

f over its entire range.

Summations in Eqs. 61 and 63 run over F values corresponding to a fixed set of paternal P and maternal M alleles actually transmitted to the offspring.

TABLE 0.15
Euploid Fetus: Parental Allele Transmission (Continued)

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 33 | A | R | R | R | PH1.MH1 | 1/2 | 1/2 | 1 − f/2 | f/2 |
| 34 | A | R | R | R | PH1.MH2 | 1/2 | 1/2 | 1 − f/2 | f/2 |
| 35 | A | R | R | R | PH2.MH1 | 1 | 0 | 1 | 0 |
| 36 | A | R | R | R | PH2.MH2 | 1 | 0 | 1 | 0 |
| 37 | A | R | R | A | PH1.MH1 | 1/2 | 1/2 | 1/2 | 1/2 |
| 38 | A | R | R | A | PH1.MH2 | 0 | 1 | (1 − f)/2 | (1 + f)/2 |
| 39 | A | R | R | A | PH2.MH1 | 1 | 0 | (1 + f)/2 | (1 − f)/2 |
| 40 | A | R | R | A | PH2.MH2 | 1/2 | 1/2 | 1/2 | 1/2 |
| 41 | A | R | A | R | PH1.MH1 | 0 | 1 | (1 − f)/2 | (1 + f)/2 |
| 42 | A | R | A | R | PH1.MH2 | 1/2 | 1/2 | 1/2 | 1/2 |
| 43 | A | R | A | R | PH2.MH1 | 1/2 | 1/2 | 1/2 | 1/2 |
| 44 | A | R | A | R | PH2.MH2 | 1 | 0 | (1 + f)/2 | (1 − f)/2 |
| 45 | A | R | A | A | PH1.MH1 | 0 | 1 | 0 | 1 |
| 46 | A | R | A | A | PH1.MH2 | 0 | 1 | 0 | 1 |
| 47 | A | R | A | A | PH2.MH1 | 1/2 | 1/2 | f/2 | 1 − f/2 |
| 48 | A | R | A | A | PH2.MH2 | 1/2 | 1/2 | f/2 | 1 − f/2 |
| 49 | A | A | R | R | PH1.MH1 | 1/2 | 1/2 | 1 − f/2 | f/2 |
| 50 | A | A | R | R | PH1.MH2 | 1/2 | 1/2 | 1 − f/2 | f/2 |
| 51 | A | A | R | R | PH2.MH1 | 1/2 | 1/2 | 1 − f/2 | f/2 |
| 52 | A | A | R | R | PH2.MH2 | 1/2 | 1/2 | 1 − f/2 | f/2 |
| 53 | A | A | R | A | PH1.MH1 | 1/2 | 1/2 | 1/2 | 1/2 |
| 54 | A | A | R | A | PH1.MH2 | 0 | 1 | (1 − f)/2 | (1 + f)/2 |
| 55 | A | A | R | A | PH2.MH1 | 1/2 | 1/2 | 1/2 | 1/2 |
| 56 | A | A | R | A | PH2.MH2 | 0 | 1 | (1 − f)/2 | (1 + f)/2 |
| 57 | A | A | A | R | PH1.MH1 | 0 | 1 | (1 − f)/2 | (1 + f)/2 |
| 58 | A | A | A | R | PH1.MH2 | 1/2 | 1/2 | 1/2 | 1/2 |
| 59 | A | A | A | R | PH2.MH1 | 0 | 1 | (1 − f)/2 | (1 + f)/2 |
| 60 | A | A | A | R | PH2.MH2 | 1/2 | 1/2 | 1/2 | 1/2 |
| 61 | A | A | A | A | PH1.MH1 | 0 | 1 | 0 | 1 |
| 62 | A | A | A | A | PH1.MH2 | 0 | 1 | 0 | 1 |
| 63 | A | A | A | A | PH2.MH1 | 0 | 1 | 0 | 1 |
| 64 | A | A | A | A | PH2.MH2 | 0 | 1 | 0 | 1 |

In some embodiments, the set Φ includes all values of transmission patterns F listed in Tables 0.8-0.19. In some embodiments, the set Φ is a non-trivial subset of values of transmission patterns F tabulated in Tables 0.8-0.19. In some embodiments, the set Φ is a non-trivial superset of values of transmission patterns F

TABLE 0.16
Fetal Trisomy: Paternal Duplication

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 1 | R | R | R | R | PH1x2.MH1 | 3/2 | 0 | 1 + f/2 | 0 |
| 2 | R | R | R | R | PH1x2.MH2 | 3/2 | 0 | 1 + f/2 | 0 |
| 3 | R | R | R | R | PH2x2.MH1 | 3/2 | 0 | 1 + f/2 | 0 |
| 4 | R | R | R | R | PH2x2.MH2 | 3/2 | 0 | 1 + f/2 | 0 |
| 5 | R | R | R | A | PH1x2.MH1 | 3/2 | 0 | 1/2 + f | (1 − f)/2 |
| 6 | R | R | R | A | PH1x2.MH2 | 1 | 1/2 | (1 + f)/2 | 1/2 |
| 7 | R | R | R | A | PH2x2.MH1 | 3/2 | 0 | 1/2 + f | (1− f)/2 |
| 8 | R | R | R | A | PH2x2.MH2 | 1 | 1/2 | (1 + f)/2 | 1/2 |
| 9 | R | R | A | R | PH1x2.MH1 | 1 | 1/2 | (1 + f)/2 | 1/2 |
| 10 | R | R | A | R | PH1x2.MH2 | 3/2 | 0 | 1/2 + f | (1 − f)/2 |
| 11 | R | R | A | R | PH2x2.MH1 | 1 | 1/2 | (1 + f)/2 | 1/2 |
| 12 | R | R | A | R | PH2x2.MH2 | 3/2 | 0 | 1/2 + f | (1− f)/2 |
| 13 | R | R | A | A | PH1x2.MH1 | 1 | 1/2 | f | 1 − f/2 |
| 14 | R | R | A | A | PH1x2.MH2 | 1 | 1/2 | f | 1 − f/2 |
| 15 | R | R | A | A | PH2x2.MH1 | 1 | 1/2 | f | 1− f/2 |
| 16 | R | R | A | A | PH2x2.MH2 | 1 | 1/2 | f | 1 − f/2 |
| 17 | R | A | R | R | PH1x2.MH1 | 3/2 | 0 | 1 + f/2 | 0 |
| 18 | R | A | R | R | PH1x2.MH2 | 3/2 | 0 | 1 + f/2 | 0 |
| 19 | R | A | R | R | PH2x2.MH1 | 1/2 | 1 | 1 − f/2 | f |
| 20 | R | A | R | R | PH2x2.MH2 | 1/2 | 1 | 1 − f/2 | f |
| 21 | R | A | R | A | PH1x2.MH1 | 3/2 | 0 | 1/2 + f | (1 − f)/2 |
| 22 | R | A | R | A | PH1x2.MH2 | 1 | 1/2 | (1 + f)/2 | 1/2 |
| 23 | R | A | R | A | PH2x2.MH1 | 1/2 | 1 | 1/2 | (1 + f)/2 |
| 24 | R | A | R | A | PH2x2.MH2 | 0 | 3/2 | (1 − f)/2 | 1/2 + f |
| 25 | R | A | A | R | PH1x2.MH1 | 1 | 1/2 | (1 + f)/2 | 1/2 |
| 26 | R | A | A | R | PH1x2.MH2 | 3/2 | 0 | 1/2 + f | (1 − f)/2 |
| 27 | R | A | A | R | PH2x2.MH1 | 0 | 3/2 | (1 − f)/2 | 1/2 + f |
| 28 | R | A | A | R | PH2x2.MH2 | 1/2 | 1 | 1/2 | (1 + f)/2 |
| 29 | R | A | A | A | PH1x2.MH1 | 1 | 1/2 | f | 1− f/2 |
| 30 | R | A | A | A | PH1x2.MH2 | 1 | 1/2 | f | 1 − f/2 |
| 31 | R | A | A | A | PH2x2.MH1 | 0 | 3/2 | 0 | 1 + f/2 |
| 32 | R | A | A | A | PH2x2.MH2 | 0 | 3/2 | 0 | 1 + f/2 |

tabulated in Tables 0.8-0.19. In some embodiments, the set Φ only includes diploid fetal transmission patterns F listed in Tables 0.14-0.15.

Most Likely Transmission Pattern Per Genomic Region

TABLE 0.17
Fetal Trisomy: Paternal Duplication (Continued)

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 33 | A | R | R | R | PH1x2.MH1 | 1/2 | 1 | 1 − f/2 | f |
| 34 | A | R | R | R | PH1x2.MH2 | 1/2 | 1 | 1 − f/2 | f |
| 35 | A | R | R | R | PH2x2.MH1 | 3/2 | 0 | 1 + f/2 | 0 |
| 36 | A | R | R | R | PH2x2.MH2 | 3/2 | 0 | 1 + f/2 | 0 |
| 37 | A | R | R | A | PH1x2.MH1 | 1/2 | 1 | 1/2 | (1 + f)/2 |
| 38 | A | R | R | A | PH1x2.MH2 | 0 | 3/2 | (1 − f)/2 | 1/2 + f |
| 39 | A | R | R | A | PH2x2.MH1 | 3/2 | 0 | 1/2 + f | (1 − f)/2 |
| 40 | A | R | R | A | PH2x2.MH2 | 1 | 1/2 | (1 + f)/2 | 1/2 |
| 41 | A | R | A | R | PH1x2.MH1 | 0 | 3/2 | (1 − f)/2 | 1/2 + f |
| 42 | A | R | A | R | PH1x2.MH2 | 1/2 | 1 | 1/2 | (1 + f)/2 |
| 43 | A | R | A | R | PH2x2.MH1 | 1 | 1/2 | (1 + f)/2 | 1/2 |
| 44 | A | R | A | R | PH2x2.MH2 | 3/2 | 0 | 1/2 + f | (1− f)/2 |
| 45 | A | R | A | A | PH1x2.MH1 | 0 | 3/2 | 0 | 1 + f/2 |
| 46 | A | R | A | A | PH1x2.MH2 | 0 | 3/2 | 0 | 1 + f/2 |
| 47 | A | R | A | A | PH2x2.MH1 | 1 | 1/2 | f | 1− f/2 |
| 48 | A | R | A | A | PH2x2.MH2 | 1 | 1/2 | f | 1− f/2 |
| 49 | A | A | R | R | PH1x2.MH1 | 1/2 | 1 | 1 − f/2 | f |
| 50 | A | A | R | R | PH1x2.MH2 | 1/2 | 1 | 1 − f/2 | f |
| 51 | A | A | R | R | PH2x2.MH1 | 1/2 | 1 | 1 − f/2 | f |
| 52 | A | A | R | R | PH2x2.MH2 | 1/2 | 1 | 1 − f/2 | f |
| 53 | A | A | R | A | PH1x2.MH1 | 1/2 | 1 | 1/2 | (1 + f)/2 |
| 54 | A | A | R | A | PH1x2.MH2 | 0 | 3/2 | (1 − f)/2 | 1/2 + f |
| 55 | A | A | R | A | PH2x2.MH1 | 1/2 | 1 | 1/2 | (1 + f)/2 |
| 56 | A | A | R | A | PH2x2.MH2 | 0 | 3/2 | (1 − f)/2 | 1/2 + f |
| 57 | A | A | A | R | PH1x2.MH1 | 0 | 3/2 | (1 − f)/2 | 1/2 + f |
| 58 | A | A | A | R | PH1x2.MH2 | 1/2 | 1 | 1/2 | (1 + f)/2 |
| 59 | A | A | A | R | PH2x2.MH1 | 0 | 3/2 | (1 − f)/2 | 1/2 + f |
| 60 | A | A | A | R | PH2x2.MH2 | 1/2 | 1 | 1/2 | (1 + f)/2 |
| 61 | A | A | A | A | PH1x2.MH1 | 0 | 3/2 | 0 | 1 + f/2 |
| 62 | A | A | A | A | PH1x2.MH2 | 0 | 3/2 | 0 | 1 + f/2 |
| 63 | A | A | A | A | PH2x2.MH1 | 0 | 3/2 | 0 | 1 + f/2 |
| 64 | A | A | A | A | PH2x2.MH2 | 0 | 3/2 | 0 | 1 + f/2 |

To determine the transmission pattern with most support from experimental data within a given genomic region, likelihoods from Eqs. 60 and 62 obtained for individual loci are combined together by multiplication or, equivalently but more conveniently, by summation of their logarithms. A region containing loci i∈1, . . . , K

TABLE 0.18
Fetal Trisomy: Maternal Duplication

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 1 | R | R | R | R | PH1.MH1x2 | 3/2 | 0 | 1 + f/2 | 0 |
| 2 | R | R | R | R | PH1.MH2x2 | 3/2 | 0 | 1 + f/2 | 0 |
| 3 | R | R | R | R | PH2.MH1x2 | 3/2 | 0 | 1 + f/2 | 0 |
| 4 | R | R | R | R | PH2.MH2x2 | 3/2 | 0 | 1 + f/2 | 0 |
| 5 | R | R | R | A | PH1.MH1x2 | 3/2 | 0 | 1/2 + f | (1 − f)/2 |
| 6 | R | R | R | A | PH1.MH2x2 | 1/2 | 1 | 1/2 | (1 + f)/2 |
| 7 | R | R | R | A | PH2.MH1x2 | 3/2 | 0 | 1/2 + f | (1 − f)/2 |
| 8 | R | R | R | A | PH2.MH2x2 | 1/2 | 1 | 1/2 | (1 + f)/2 |
| 9 | R | R | A | R | PH1.MH1x2 | 1/2 | 1 | 1/2 | (1 + f)/2 |
| 10 | R | R | A | R | PH1.MH2x2 | 3/2 | 0 | 1/2 + f | (1 − f)/2 |
| 11 | R | R | A | R | PH2.MH1x2 | 1/2 | 1 | 1/2 | (1 + f)/2 |
| 12 | R | R | A | R | PH2.MH2x2 | 3/2 | 0 | 1/2 + f | (1 − f)/2 |
| 13 | R | R | A | A | PH1.MH1x2 | 1/2 | 1 | f/2 | 1 |
| 14 | R | R | A | A | PH1.MH2x2 | 1/2 | 1 | f/2 | 1 |
| 15 | R | R | A | A | PH2.MH1x2 | 1/2 | 1 | f/2 | 1 |
| 16 | R | R | A | A | PH2.MH2x2 | 1/2 | 1 | f/2 | 1 |
| 17 | R | A | R | R | PH1.MH1x2 | 3/2 | 0 | 1 + f/2 | 0 |
| 18 | R | A | R | R | PH1.MH2x2 | 3/2 | 0 | 1 + f/2 | 0 |
| 19 | R | A | R | R | PH2.MH1x2 | 1 | 1/2 | 1 | f/2 |
| 20 | R | A | R | R | PH2.MH2x2 | 1 | 1/2 | 1 | f/2 |
| 21 | R | A | R | A | PH1.MH1x2 | 3/2 | 0 | 1/2 + f | (1 − f)/2 |
| 22 | R | A | R | A | PH1.MH2x2 | 1/2 | 1 | 1/2 | (1 + f)/2 |
| 23 | R | A | R | A | PH2.MH1x2 | 1 | 1/2 | (1 + f)/2 | 1/2 |
| 24 | R | A | R | A | PH2.MH2x2 | 0 | 3/2 | (1 − f)/2 | 1/2 + f |
| 25 | R | A | A | R | PH1.MH1x2 | 1/2 | 1 | 1/2 | (1 + f)/2 |
| 26 | R | A | A | R | PH1.MH2x2 | 3/2 | 0 | 1/2 + f | (1 − f)/2 |
| 27 | R | A | A | R | PH2.MH1x2 | 0 | 3/2 | (1 − f)/2 | 1/2 + f |
| 28 | R | A | A | R | PH2.MH2x2 | 1 | 1/2 | (1 + f)/2 | 1/2 |
| 29 | R | A | A | A | PH1.MH1x2 | 1/2 | 1 | f/2 | 1 |
| 30 | R | A | A | A | PH1.MH2x2 | 1/2 | 1 | f/2 | 1 |
| 31 | R | A | A | A | PH2.MH1x2 | 0 | 3/2 | 0 | 1 + f/2 |
| 32 | R | A | A | A | PH2.MH2x2 | 0 | 3/2 | 0 | 1 + f/2 |

characterized by known and phased parental alleles P(i) and M(i) and by measured fetal allele frequencies n_{R}(i) and n_{A}(i) is thus assigned the following set of

TABLE 0.19
Fetal Trisomy: Maternal Duplication (Continued)

| # | PH1 | PH2 | MH1 | MH2 | F | [symbol] _{R} (F) | [symbol] _{A} (F) | θ_{R} (F, f) | θ_{A} (F, f) |
|---|---|---|---|---|---|---|---|---|---|
| 33 | A | R | R | R | PH1.MH1x2 | 1 | 1/2 | 1 | f/2 |
| 34 | A | R | R | R | PH1.MH2x2 | 1 | 1/2 | 1 | f/2 |
| 35 | A | R | R | R | PH2.MH1x2 | 3/2 | 0 | 1 + f/2 | 0 |
| 36 | A | R | R | R | PH2.MH2x2 | 3/2 | 0 | 1 + f/2 | 0 |
| 37 | A | R | R | A | PH1.MH1x2 | 1 | 1/2 | (1 + f)/2 | 1/2 |
| 38 | A | R | R | A | PH1.MH2x2 | 0 | 3/2 | (1 − f)/2 | 1/2 + f |
| 39 | A | R | R | A | PH2.MH1x2 | 3/2 | 0 | 1/2 + f | (1 − f)/2 |
| 40 | A | R | R | A | PH2.MH2x2 | 1/2 | 1 | 1/2 | (1 + f)/2 |
| 41 | A | R | A | R | PH1.MH1x2 | 0 | 3/2 | (1 − f)/2 | 1/2 + f |
| 42 | A | R | A | R | PH1.MH2x2 | 1 | 1/2 | (1 + f)/2 | 1/2 |
| 43 | A | R | A | R | PH2.MH1x2 | 1/2 | 1 | 1/2 | (1 + f)/2 |
| 44 | A | R | A | R | PH2.MH2x2 | 3/2 | 0 | 1/2 + f | (1 − f)/2 |
| 45 | A | R | A | A | PH1.MH1x2 | 0 | 3/2 | 0 | 1 + f/2 |
| 46 | A | R | A | A | PH1.MH2x2 | 0 | 3/2 | 0 | 1 + f/2 |
| 47 | A | R | A | A | PH2.MH1x2 | 1/2 | 1 | f/2 | 1 |
| 48 | A | R | A | A | PH2.MH2x2 | 1/2 | 1 | f/2 | 1 |
| 49 | A | A | R | R | PH1.MH1x2 | 1 | 1/2 | 1 | f/2 |
| 50 | A | A | R | R | PH1.MH2x2 | 1 | 1/2 | 1 | f/2 |
| 51 | A | A | R | R | PH2.MH1x2 | 1 | 1/2 | 1 | f/2 |
| 52 | A | A | R | R | PH2.MH2x2 | 1 | 1/2 | 1 | f/2 |
| 53 | A | A | R | A | PH1.MH1x2 | 1 | 1/2 | (1 + f)/2 | 1/2 |
| 54 | A | A | R | A | PH1.MH2x2 | 0 | 3/2 | (1 − f)/2 | 1/2 + f |
| 55 | A | A | R | A | PH2.MH1x2 | 1 | 1/2 | (1 + f)/2 | 1/2 |
| 56 | A | A | R | A | PH2.MH2x2 | 0 | 3/2 | (1 − f)/2 | 1/2 + f |
| 57 | A | A | A | R | PH1.MH1x2 | 0 | 3/2 | (1 − f)/2 | 1/2 + f |
| 58 | A | A | A | R | PH1.MH2x2 | 1 | 1/2 | (1 + f)/2 | 1/2 |
| 59 | A | A | A | R | PH2.MH1x2 | 0 | 3/2 | (1 − f)/2 | 1/2 + f |
| 60 | A | A | A | R | PH2.MH2x2 | 1 | 1/2 | (1 + f)/2 | 1/2 |
| 61 | A | A | A | A | PH1.MH1x2 | 0 | 3/2 | 0 | 1 + f/2 |
| 62 | A | A | A | A | PH1.MH2x2 | 0 | 3/2 | 0 | 1 + f/2 |
| 63 | A | A | A | A | PH2.MH1x2 | 0 | 3/2 | 0 | 1 + f/2 |
| 64 | A | A | A | A | PH2.MH2x2 | 0 | 3/2 | 0 | 1 + f/2 |

log-likelihood values:

$$ln ℒ ( F ) = {\sum }_{i = 1}^{K} ln ℙ ( {n}_{R} ( i ) , {n}_{A} ( i ) \vert F ( i ) , P ( i ) , M ( i ) ) , F ∈ Φ \qquad ( 64 )$$

The result is a set of likelihood (or log-likelihood) values for the entire genomic region, one value per F pattern from the set Φ. The most likely pattern F₀ is the one with the highest likelihood:

$${F}_{0} = {arg max}_{F ∈ Φ} ( ℒ ( F ) )$$

Region Homogeneity

The estimate F₀ may contradict some loci, where the locus-specific likelihood values supply more support a scenario different from F₀. If all loci in a region have likelihood distributions [symbol] (n_{R}(i),n_{A}(i)|F(i),P(i),M(i)) such that none lands more support to F≠F₀, the region is homogeneous. A homogeneous region contains a single haplotype block from each parent (in a diploid scenario).

Some loci may be uninformative, in which case [symbol] (n_{A},n_{A}|F≠F₀,P,M) may be equal to [symbol] (n_{R},n_{A}|F₀,P,M) without violating the homogeneity requirement. If at least one locus in a region lands more support to a transmission pattern F≠F₀ (meaning that [symbol] (n_{R},n_{A}|F≠F₀,P,M)> [symbol] (n_{R},n_{A}|F₀,P,M)), the region is inhomogeneous. An inhomogeneous region contains two or more haplotype blocks inherited from one or both parents.

Region Segmentation

The goal of the analysis is to partition the fetal genome into homogeneous regions. Many algorithms to effectively partition a region are known to those skilled in arts, including but not limited to bisection, golden ratio section, and partitioning at the first or the last locus contradicting the selection of most likely regional pattern F₀. Various approaches differ in terms of time and memory complexity, but all eventually converge to a set of maximally sized homogeneous subregions, representing unbroken inherited haplotype blocks.

Recombination Breakpoints

In addition to segmentation, the determination of parental haplotype inheritance patterns also includes a merging step. If, for example, the most likely transmission patterns F₀ in two neighboring homogeneous subregions are PH1.MH1 and PH1.MH2, it is likely that PH1 continues unbroken through the interval separating the two subregions and covers them both. In that case, the gap separating the two homogeneous subregions represents the maternal recombination event where M switches from MH1 to MH2.

Because of the distance between neighboring loci in the SNP panel and because of the unavoidable presence of uninformative loci, the intervals separating neighboring homogeneous subregions may be several kilobases long. The recombination breakpoints are somewhere within these intervals, but the data are insufficient to indicate where exactly. To avoid overinterpreting the data, the final haplotype edge calls coincide with the first and the last informative loci within a homogeneous block. The intervals separating neighboring haplotypes represent uncertainties in locations of recombination events. Detection of transmission of a monogenic disease associated with these uncertainty intervals requires additional information, such as a more dense set of SNPs.

### K. Validation Data: Single Cell Cancer Copy Number Profiles

To validate algorithms intended for analyses of cancer CNV profiles, a list of human cell lines available from ATCC (web page https://www.atcc.org/search?title=ATCC%20Human%20Cell%20Lines%20-%20Alphanumeric#q=%40productline%3DC021&sort=relevancy&f:contentTypeFacetATCC=[Products]) was compiled and searched for copy number profile availability at Institute for Cancer Research (ICR, cf. https://cansarblack.icr.ac.uk/).

The copy number profiles of the resulting 438 cell lines were downloaded from ICR and used as templates for simulating whole genome resequencing data at different depths and GC bias levels. The simulated profiles were then used to validate the proposed algorithms and to characterize their performance.

### L. Reduction to Practice of the System and Method to Estimate GC Bias Using Fourier Coefficient Ratio and Differential Variance Ratio as Applied to Simulated Data

The proposed method to estimate GC bias coefficients in cancerous CNV profiles using Fourier Coefficient Ratio is tested on 1,000 simulated raw count profiles based on the ICR ground truth for the cell line 22Rv-1. The performance of the Fourier Coefficient Ratio approach is compared to results obtained using Željko Džakula's earlier invention based on minimization of entropy.

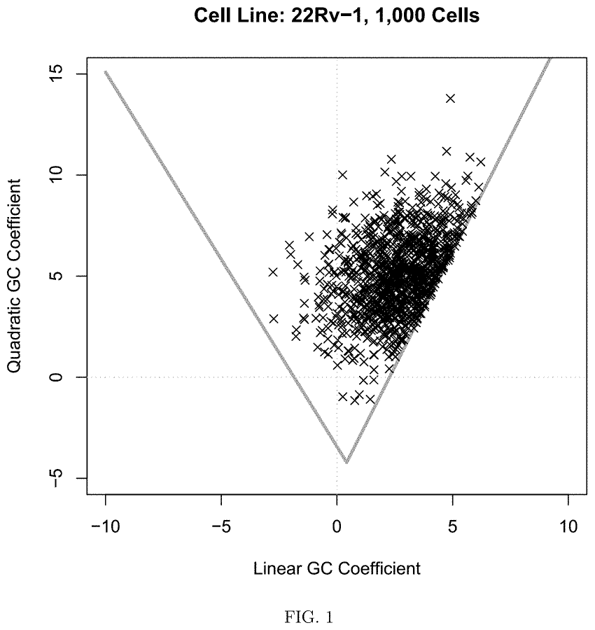

For each cell, a raw count profile was initiated by uniformly sampling the scale between 50 and 100 reads per 50 kb bin (roughly equivalent to 3-6 million reads per cell). Next, to generate linear (L) and quadratic (Q) GC bias coefficients, the center of the two dimensional (L,Q) distribution was randomly chosen. The mean linear coefficient was uniformly sampled between −4 and 4. The mean quadratic coefficient was then uniformly sampled. The lower bound for uniform sampling of the mean quadratic coefficient is imposed by the previously selected mean linear coefficient value and the non-negativity condition. The non-negativity is enforced for g_{L}=0.01, g_{U}=0.99, and g₀=0.45. The upper bound is arbitrarily set to 6. Once the center of the two dimensional distribution of linear and quadratic GC coefficients is fixed, the (L,Q) distribution is defined as the two dimensional Gaussian with standard deviations along both L and Q dimensions set to 2 and the covariance fixed at 0.5. Actual GC coefficient values for each individual cell are sampled from this two dimensional Gaussian, making sure that the non-negativity constraint is satisfied. FIG. 1 shows the resulting linear and quadratic GC coefficient values per cell.

To simulate raw bin counts, GC content per bin values were evaluated based on human reference sequence hg19 using Bioconductor. Mappability values were extracted from the file wgEncodeCrgMapabilityAlign100mer.bigWig (downloaded from UCSC Genome Browser http://hgdownload.soe.ucsc.edu/goldenPath/hg19/encodeDCC/wgEncodeMapability/) on Mar. 16, 2020).

For each cell, raw counts per bin were sampled using Poisson distribution centered at PSmG(g,L,Q,g₀). The ploidy values P are taken from the ICR ground truth profile. Scale S, linear GC coefficient L, and quadratic coefficient Q are sample-specific. Mappability m and GC content g are bin-specific and constant across samples. The constant g₀=0.45 is arbitrarily chosen and fixed for all calculations.

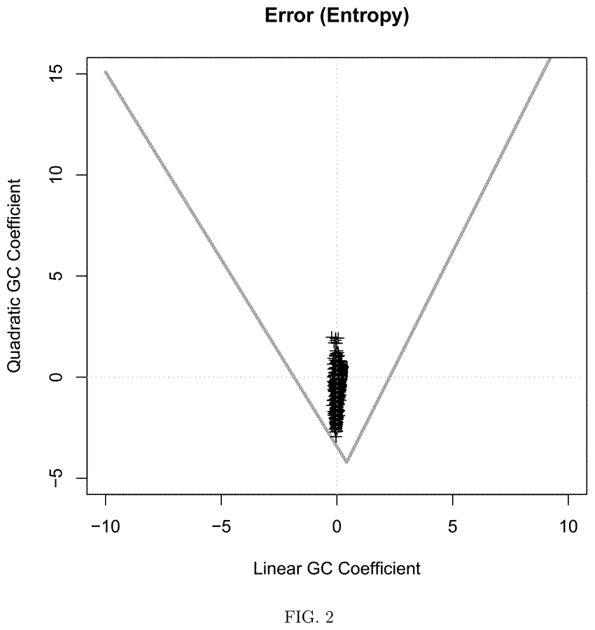

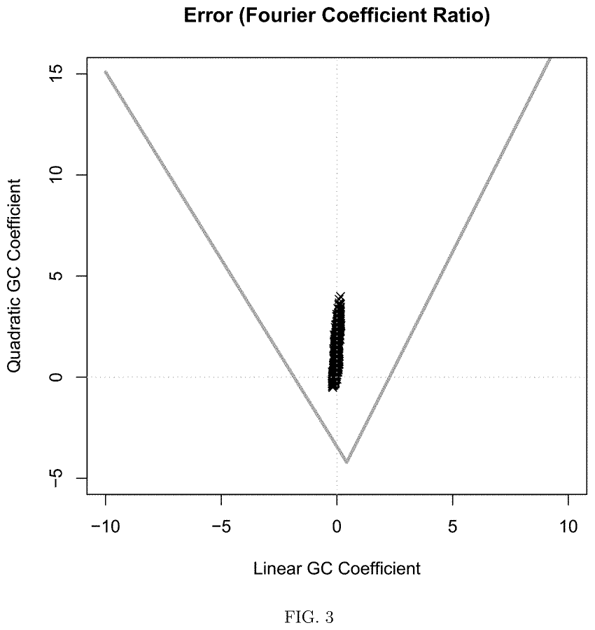

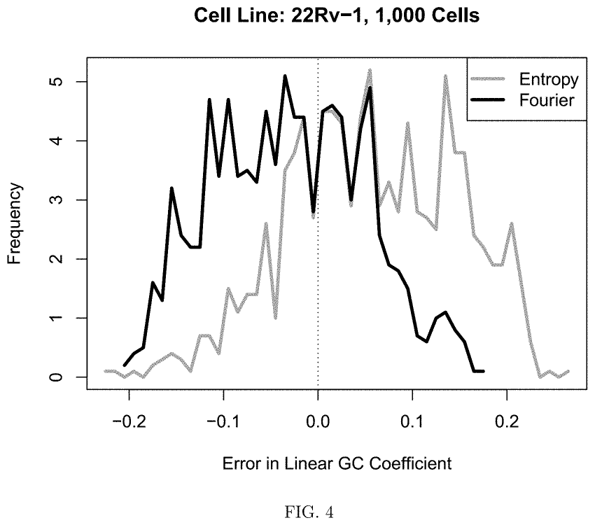

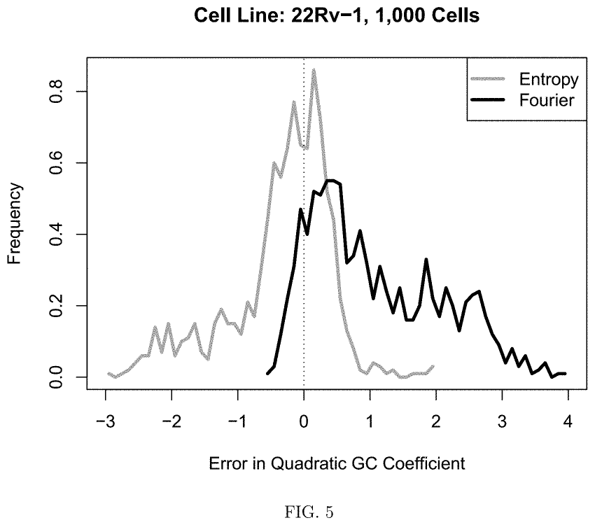

Linear and quadratic GC coefficients are estimated using entropy and Fourier Coefficient Ratio as described above. The residual errors (estimated minus true coefficient values) observed when entropy is used are illustrated in FIG. 2. FIG. 3 shows the distribution of residual errors obtained with Fourier Coefficient Ratio. FIG. 2 and FIG. 3 are plotted with the same axis ranges as FIG. 1 to illustrate the extent of improvement relative to unnormalized counts. The distributions of errors in linear GC coefficient are shown in FIG. 4 both for entropy (gray curve) and for Fourier Coefficient Ratio (black curve). The FRC distribution is centered closer to zero than the entropy curve. FIG. 5 presents distributions of observed errors in quadratic coefficients both for entropy (gray) and FRC-based estimation (black). The two algorithms display opposite behavior: while errors in entropy-based Q-values have strong left tail, the errors in FRC-derived quadratic coefficients are skewed to the right.

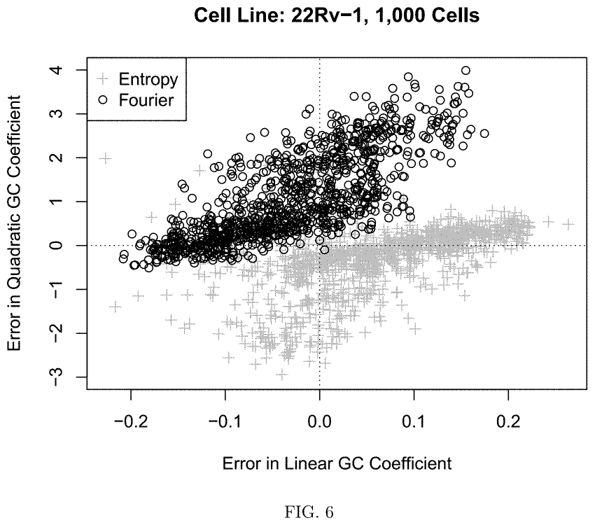

Tbl. 0.20 characterizes errors in linear and quadratic GC coefficient estimates obtained using Entropy (column 2) or Fourier Coefficient Ratio (column 3). Rows A-B list mean and median errors in linear GC coefficients. Rows C-D show standard deviation and MAD values for errors in linear coefficients. Rows E-F show mean and median errors in quadratic GC coefficients. Rows G-H list standard deviation and MAD of errors in quadratic coefficients. Finally, row I shows correlation coefficients between errors in linear and quadratic coefficients (also illustrated in FIG. 6).

As a rule, Fourier Coefficient Ratio makes smaller errors than entropy-based approach when estimating linear GC bias coefficients, and FRC's errors are more tightly distributed. On average, errors in quadratic GC bias coefficient estimates are larger when Fourier Coefficient Ratio is used, and their distribution is more widely spread. Both entropy and Fourier Coefficient Ratio show correlation between errors in linear and quadratic coefficients. The correlation is weaker when entropy is used.

Once GC coefficients L and Q are known, the unevenness in raw bin counts can be reduced by division with mappability m and with the GC bias function

TABLE 0.20
Errors in Estimated GC Bias Coefficients.

| Descriptor* | Entropy | Fourier Coefficient Ratio |
|---|---|---|
| A | 0.0579 | −0.0298 |
| B | 0.0550 | −0.0304 |
| C | 0.0880 | 0.0797 |
| D | 0.1015 | 0.0930 |
| E | −0.3360 | 1.0740 |
| F | −0.1745 | 0.8250 |
| G | 0.7705 | 0.9777 |
| H | 0.5612 | 1.0007 |
| I | 0.4971 | 0.7738 |

*Row names:
1. A: Mean Error in Linear GC Coefficient
2. B: Median Error in Linear GC Coefficient
3. C: Standard Deviation of Error in Linear GC Coefficient
4. D: MAD of Error in Linear GC Coefficient
5. E: Mean Error in Quadratic GC Coefficient
6. F: Median Error in Quadratic GC Coefficient
7. G: Standard Deviation of Error in Quadratic GC Coefficient
8. H: MAD of Error in Quadratic GC Coefficient
9. I: Correlation Coefficient (Error in Linear vs. Error in Quadratic GC Coefficient)

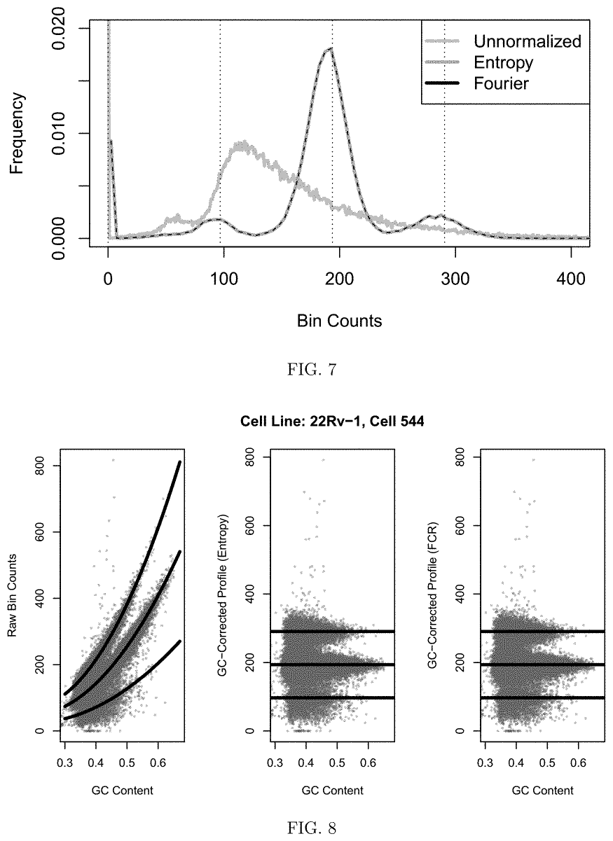

G(g,L,Q,g₀). The effects of this normalization are illustrated on one cell with extremely high GC bias (L>5, Q>10). FIG. 7 shows the distributions of bin counts in cell 544 before and after division with mG(g,L,Q,g₀). The unnormalized distribution (gray curve) is broad, with overlapping peaks that make different ploidy values indistinguishable. Both entropy-based and FCR-based normalized bin counts (black and dark-gray curves) feature much narrower peaks with minimal overlaps. Entropy and FCR yield nearly identical distributions, not discernible by visual inspection.

FIG. 8 shows bin counts for the same cell 544, plotted against GC content per bin before (left inset) and after normalization (center and right insets). While unnormalized counts show strong dependence on GC content, the count-vs-GC trend is practically eliminated when entropy (center) or FCR (right inset) is used to estimate the coefficients and to normalize the profile.

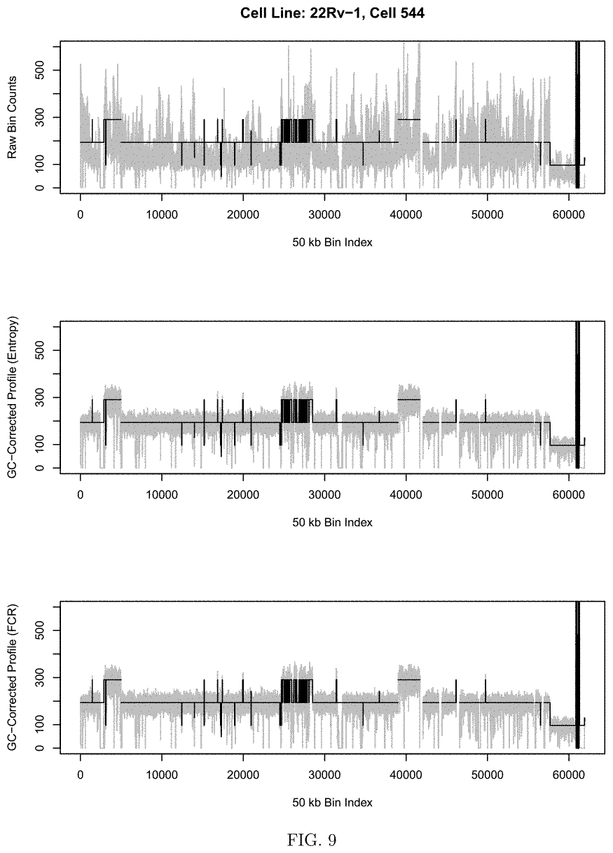

Finally, FIG. 9 shows unnormalized profile of 22Rv-1 cell 544 before normalization (top inset, gray curve) and after normalization (center and bottom insets, gray curves). Black curves show the copy number ground truth as downloaded from ICR and used as starting point in simulating read counts.

These results demonstrate that Fourier Coefficient Ratio is capable of removing GC bias from CNV profiles at least as successfully as entropy.

### M. Reduction to Practice of the System and Method to Estimate Posterior Ploidy Probability as Applied to Simulated Single Cell Resequencing Data

To test the CNV profile normalization algorithm based on posterior ploidy probability, ICR copy-number profiles for 231 cell lines were used as starting templates for simulated raw bin counts and as ground truth when assessing performance. The number of simulated cells per cell line varied from 75-76 (183 cell lines) to 696-699 (26 cell lines) to 1,000 (21 cell lines), also including one cell line with 742 cells. The total number of cells used for validation is 53,776.

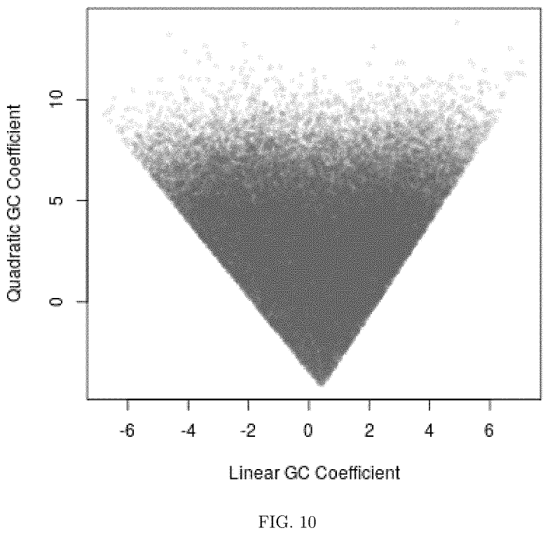

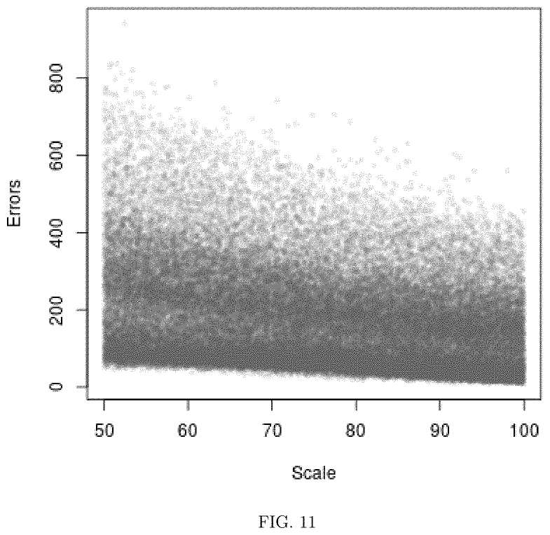

Simulated read counts used uniformly sampled scale values S that ranged between 50 and 100. GC coefficient values L and Q were randomly generated within the entire permissible V-shaped region spanning the linear GC coefficient values from −6.8 to 7.1 and quadratic coefficient values from −4.1 to 13.8 (cf. FIG. 10). (L,Q) value pairs below this V-shaped area would violate non-negativity condition and were excluded from simulations. The simulated GC bias values range from complete absence of bias to extremely biased scenarios usually not observed in measured data, ensuring coverage of all representative cases.

Raw counts per 50 kb bin were generated using Poisson distribution with the mean set to PSmG(g,L,Q,g₀), (P=bin ploidy, S=scale, m=mappability, g=GC content per bin, L=linear GC coefficient, Q=quadratic GC coefficient, g₀=0.45, and G is a parabola describing the GC bias). Bins with mappability below 0.90, GC content lower than 0.3, or GC content above 0.7 were filtered out, leaving 52,437 useful bins (down from 61,927 total). Posterior probability for bin ploidies used uniform count prior. The ploidy prior was set to a mixture of uniform (0.1%) and ploidy posterior for the previous bin (99.9%), except for the first bin, where the ploidy prior was uniform. Final ploidy values were estimated from the locations of maxima of the posterior ploidy distributions per bin.

To separate inaccuracies in GC coefficient estimation from performance inherent to posterior ploidy probability algorithm, the validation used true L and Q values, rather than estimates based on entropy or Fourier Coefficient Ratio. A separate study focuses on the impact of errors in L and Q on normalized profile accuracy.

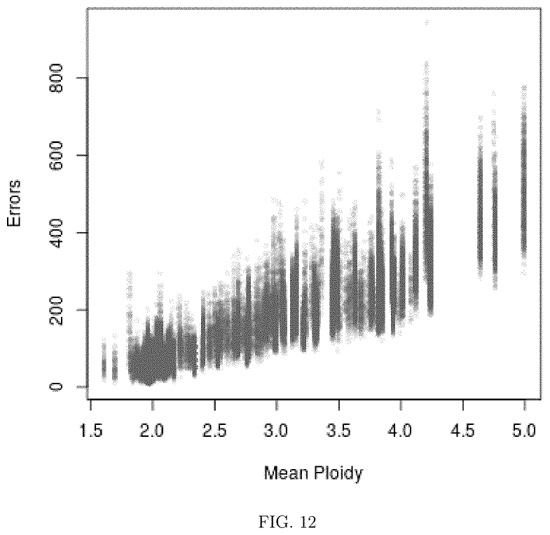

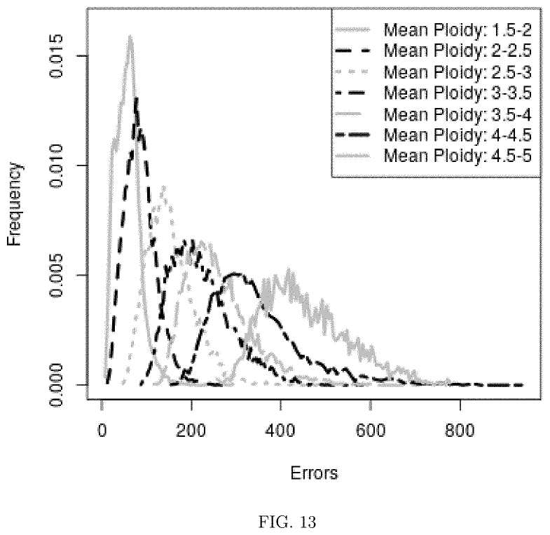

Performance of the posterior ploidy probability mode estimates was quantified by counting bins where the location of the mode differs from ground truth ploidy by more than 0.5. The number of errors per cell decreases with increasing scale S (FIG. 11) and with increasing mean cell ploidy (FIG. 12). FIG. 13 shows the distributions of errors per cell after classifying cells into seven groups according to their mean ploidies. Even at lowest scale S and highest mean ploidy, error count per cell does not exceed 1,000. Since the number of useful bins (mappability above 0.9, GC content between 0.3 and 0.7) is 52,437, the relative error remains below 2% in all 53,776 cells. Increased number of errors for cells with higher mean ploidies is to be expected for two reasons: 1) higher mean ploidy means fewer reads per ploidy unit, and 2) at higher elevations, Poisson variance linearly increases with ploidy, such that stochastic error exceeds the gap between two successive ploid y values.

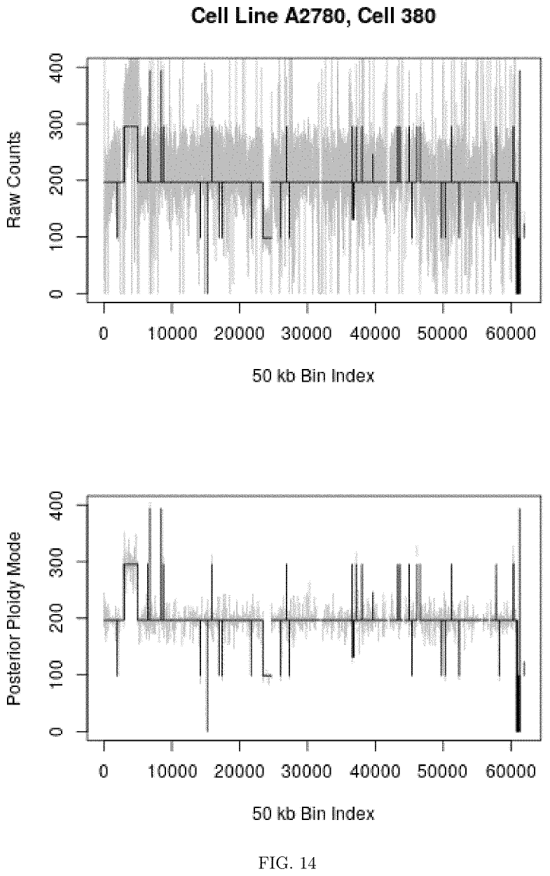

FIG. 14 demonstrates the reduction in noise after applying posterior ploidy probability-based normalization (cell A2780, cell line 380)

### N. Reduction to Practice of the System and Method to Estimate Scale as Applied to Simulated Single Cell Resequencing Data

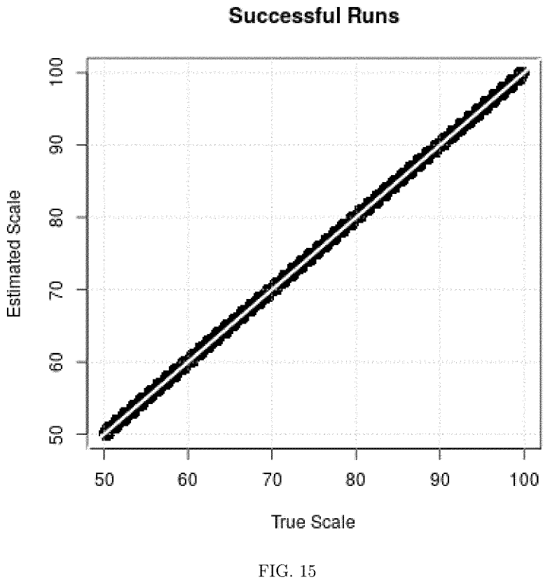

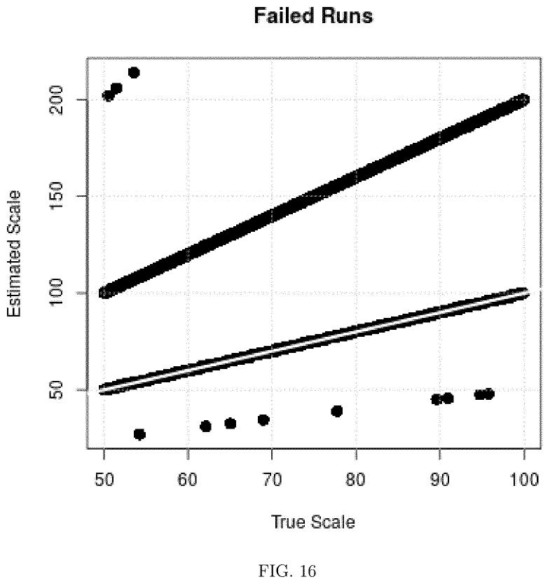

The scaling procedure is validated using the same 231 cell lines (55,260 cells) previously used to validate normalization by posterior ploidy probability. The procedure was successful in 213 cell lines (45,879 cells, FIG. 15). Scaling failed at least once in 18 cell lines (see FIG. 16).

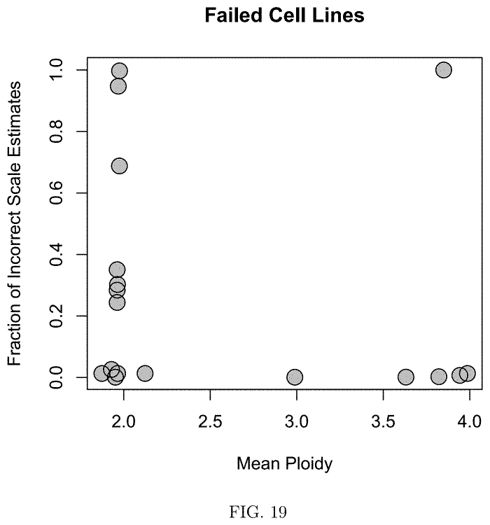

Among the 18 failed cell lines, only a fraction of cells gives incorrect results (2,898 out of 9,381 cells total). Most failed cell lines are characterized by mean ploidy close to 2 or 4, as illustrated by Table 0.21, FIG., and FIG. 19. In 7 cell lines, scaling fails only in one out of at least 76 cells. In additional two cell lines, scaling fails in 2 or 5 cells, respectively.

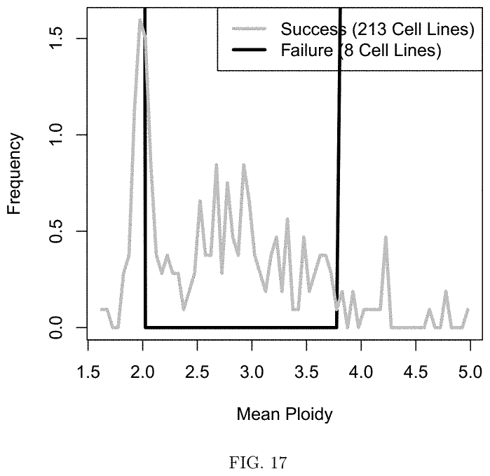

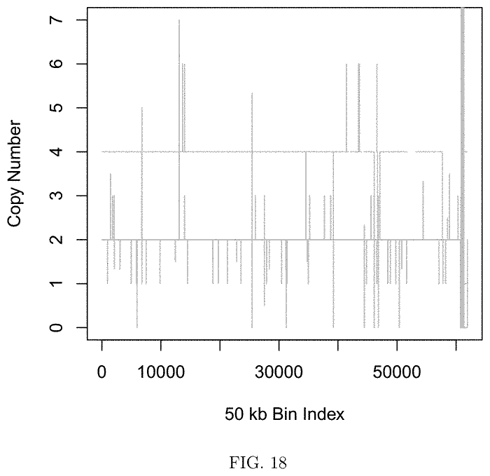

FIG. 17 shows the distributions of mean ploidies per cell line for cell lines that failed scaling (black curve) and for those that were successfully scaled. Failures on a large scale are only seen in cell lines with mean ploidy close to an even integer

TABLE 0.21
Cell Lines with Failed Scaling.

| Index | CellLine | Errors | Total | MeanPloidy |
|---|---|---|---|---|
| 1 | 5637 | 1 | 1,000 | 2.9886 |
| 2 | A-673 | 1 | 1,000 | 1.9516 |
| 3 | CML-T1 | 1 | 76 | 1.9644 |
| 4 | IGROV-1 | 1 | 76 | 3.9866 |
| 5 | J82 | 1 | 746 | 3.6311 |
| 6 | LOVO | 1 | 76 | 2.1230 |
| 7 | MC-IXC | 1 | 76 | 1.8734 |
| 8 | HOP-92 | 2 | 742 | 3.8213 |
| 9 | KASUMI-1 | 2 | 76 | 1.9288 |
| 10 | CCF-STTG1 | 5 | 720 | 3.9426 |
| 11 | MOLT-16 | 23 | 76 | 1.9635 |
| 12 | IM-9 | 72 | 76 | 1.9682 |
| 13 | ARPE-19 | 175 | 717 | 1.9616 |
| 14 | EMPTY | 208 | 732 | 1.9616 |
| 15 | EUPLOID_FEMALE | 256 | 730 | 1.9616 |
| 16 | A-204 | 688 | 1,000 | 1.9748 |
| 17 | DAUDI | 729 | 729 | 3.8495 |
| 18 | FARAGE | 731 | 733 | 1.9754 |

Summary of cell lines with failed scaling. Column “Errors” counts cells where scale estimate significantly differs from true value. Column “Total” shows total number of cells per cell line.

(2 or 4). Among cell lines with mean ploidy close to 2, most were scaled correctly, and only a minority failed. The cell line DAUDI has mean ploidy close to 3.85, yet all its cells failed the scaling; the reason is that this cell line's copy number profile mostly consists of segments with even ploidy values, with negligible number of bins at odd elevations.

As a rule, cell lines where scaling failed have very few bins that deviate from ploidy 2, as illustrated in FIG. 18. Prior knowledge that these cell lines are nearly diploid would allow division by 2, resulting in correct scale estimates.

O. Reduction to Practice of the System and Method to Determine Posterior Probability of Fetal Ploidy and Fetal Fraction in ccfDNA

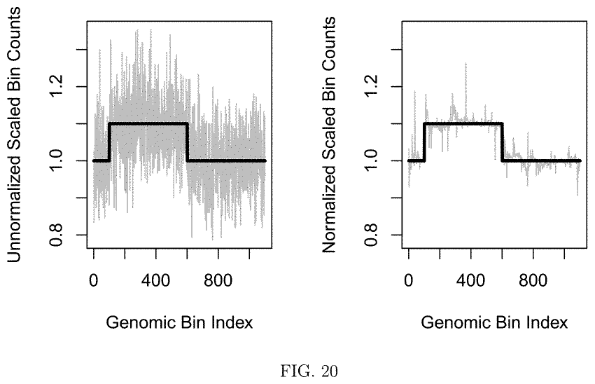

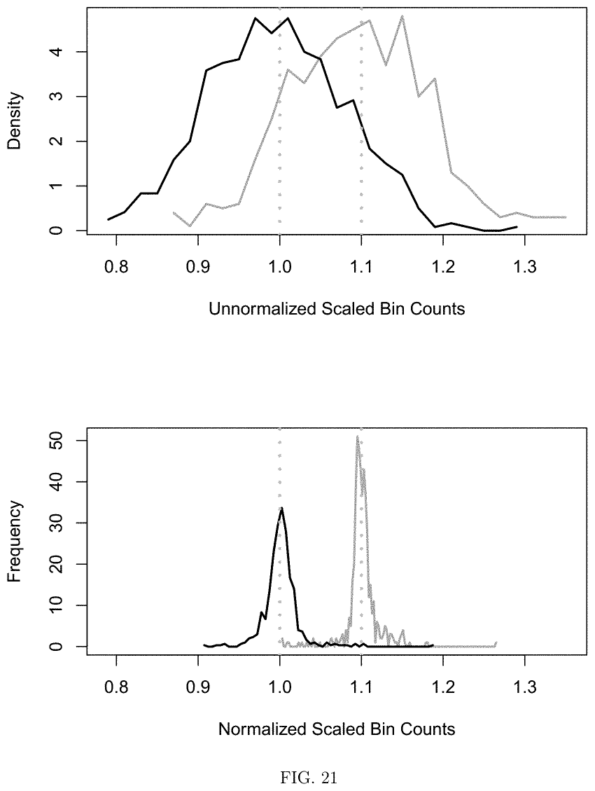

The proposed method to extract posterior probability of combined maternal and fetal ploidy from ccfDNA whole genome resequencing data was tested on simulated ccfDNA copy number profiles. FIG. 20 shows a segment of one such simulated profile containing a fetal duplication. The right inset shows that the proposed normalization based on posterior probabilities greatly reduces the amount of noise relative to the raw counts (left inset). FIG. 21 shows the distributions of bin counts in the same segment, before and after applying posterior probability-based normalization. Distributions of unnormalized counts (top inset) originating from euploid and trisomy regions show significant overlap. After normalization (bottom inset) most of the overlap is eliminated, enabling clear discrimination between affected and unaffected segments.

### P. Reduction to Practice of the System and Method to Segment Single Cell CNV Profiles Using Fourier Coefficient Ratio and Differential Variance as Applied to Simulated Single Cell Resequencing Data

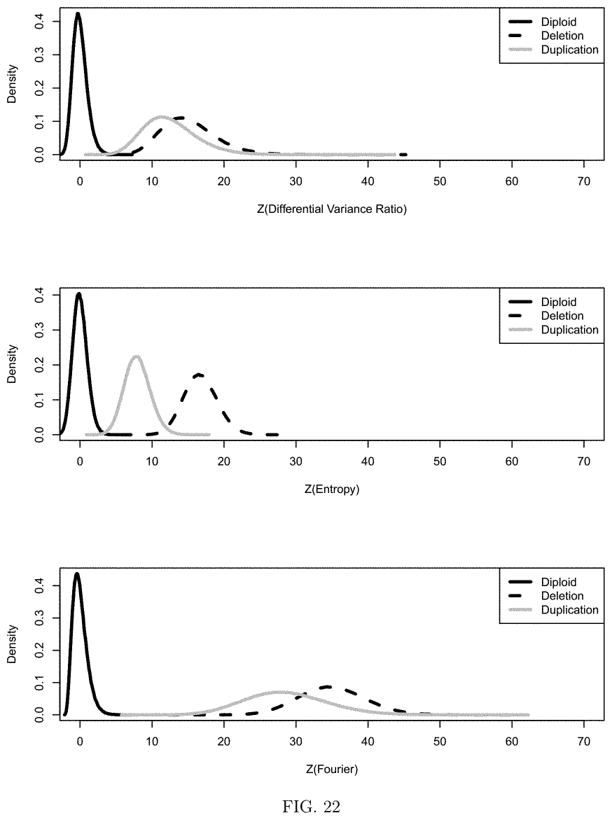

FIG. 22 demonstrates the advantages of using Fourier Coefficient Ratio (FCR) as the metric for profile homogeneity. The three insets show the performance of Differential Variance Ratio (DVR, top inset), Entropy (center), and Fourier Coefficient Ratio (bottom) in distinguishing deletions and duplications from diploid profiles. To facilitate comparisons between the three metrics, all three are standardized with respect to their corresponding diploid distributions by subtracting the observed diploid mean and then dividing the difference with the observed diploid standard deviation.

Since the dashed curve in the middle inset in FIG. 22 is shifted to the right relative to the dashed curve on the top, entropy discriminates deletions from euploid profiles better than Differential Variance Ratio. On the other hand, gray curves suggest that duplications are easier to detect with DVR than with entropy. In general, heterozygous duplications are more difficult to detect than deletions. DVR therefore has slight advantage over entropy when CNV detection is concerned. However, Fourier Coefficient Ratio (bottom inset) shifts both duplications and deletions far to the right relative to DVR and entropy, while maintaining the same diploid distribution. The significantly increased gap between affected and euploid distributions implies higher sensitivity and higher specificity when using FCR, relative to both DVR and entropy.

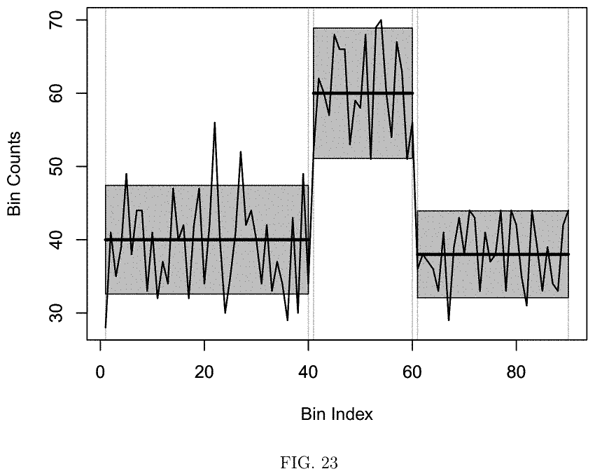

Any profile segmentation technique known to those skilled in the art, including but not limited to bisection, golden ratio section, partitioning at spikes of the differential profile, and simple bin-by-bin crawling can make use of DVR, entropy, or FCR as the homogeneity metric when detecting CNVs. FIG. 23 demonstrates the use of FCR combined with a bisection-based segmentation algorithm to partition a profile containing a heterozygous duplication.

### Q. Reduction to Practice of the System and Method to Cluster Single Cell CNV Profiles Using Fourier Coefficient Ratio and Differential Variance as Applied to Simulated Single Cell Resequencing Data

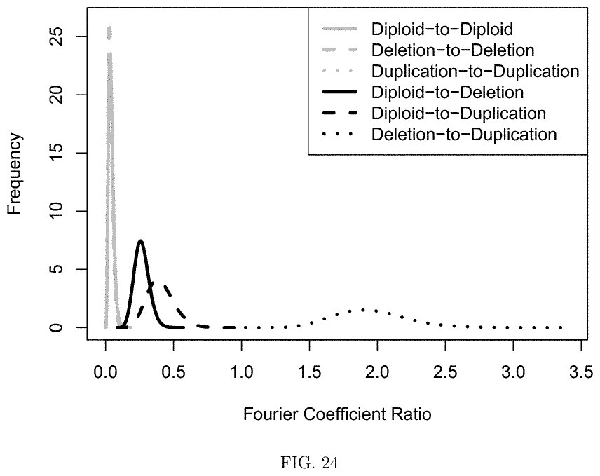

Fourier Coefficient Ratio values obtained from pairwise differences of similar copy-number profiles follow a tight distribution centered near origin. Distributions of FCR values evaluated on pairwise differences of dissimilar profiles are shifted to the right from the origin. The extent of the shift depends on the amount of dissimilarity, as illustrated by FIG. 24. To avoid division by small numbers, each pairwise profile difference is translated upward by one. The gap between the distributions obtained for comparisons of similar profiles (gray curves) and those obtained by comparing dissimilar profiles (black curves) demonstrates the benefits of using FCR as the dissimilarity measure when clustering cells according to their CNV profiles.

### R. Reduction to Practice of the System and Method to Estimate Contamination as Applied to Simulated Allele Counts

The proposed algorithms that use allele frequencies as inputs are validated using simulated data based on a set of 65 human biallelic SNP loci with high minor allele frequency. Table 0.22 shows the SNP panel used to generate allele counts. Genomic coordinates correspond to human reference GRCh38. To validate the algorithm that quantifies contamination in a mixture of sample and impurity contributions, 1,000 data sets were simulated. The total number of reads per sample uniformly varies from 30,000 to 80,000. The amount of contaminating DNA uniformly varies from 0 to 0.25. In any sample, a subset of loci fails (“drop out”). For different samples, the fraction of failed loci follows a uniform distribution from 0 to 0.04. A given locus is assigned a capacity to absorb certain fraction of the total number of reads in the sample (last column in Table 0.22, labeled “Depth Fraction”). Actual number of reads per locus is generated as a Poisson random variable with mean equal to the product of total reads per sample and the depth fraction characteristic for the locus in question. Read counts are apportioned to the two alleles according to the relationships listed in [symbol] . ??.

TABLE 0.22
SNP Panel.

| Index | MAF | Depth Fraction |
|---|---|---|
| 1 | 0.4706 | 0.0036 |
| 2 | 0.3674 | 0.0048 |
| 3 | 0.3165 | 0.0055 |
| 4 | 0.4748 | 0.0060 |
| 5 | 0.3730 | 0.0062 |
| 6 | 0.4545 | 0.0064 |
| 7 | 0.2772 | 0.0067 |
| 8 | 0.3686 | 0.0070 |
| 9 | 0.4880 | 0.0074 |
| 10 | 0.4099 | 0.0080 |
| 11 | 0.4547 | 0.0086 |
| 12 | 0.4956 | 0.0086 |
| 13 | 0.4229 | 0.0094 |
| 14 | 0.4557 | 0.0095 |
| 15 | 0.2800 | 0.0096 |
| 16 | 0.3464 | 0.0097 |
| 17 | 0.3181 | 0.0098 |
| 18 | 0.4659 | 0.0106 |
| 19 | 0.4960 | 0.0109 |
| 20 | 0.4127 | 0.0110 |
| 21 | 0.4615 | 0.0112 |
| 22 | 0.4237 | 0.0114 |
| 23 | 0.4926 | 0.0116 |
| 24 | 0.3081 | 0.0125 |
| 25 | 0.4573 | 0.0125 |
| 26 | 0.4491 | 0.0127 |
| 27 | 0.3429 | 0.0127 |
| 28 | 0.2841 | 0.0128 |
| 29 | 0.4064 | 0.0129 |
| 30 | 0.4008 | 0.0138 |
| 31 | 0.3866 | 0.0139 |
| 32 | 0.3538 | 0.0140 |
| 33 | 0.4333 | 0.0141 |
| 34 | 0.4828 | 0.0142 |
| 35 | 0.3253 | 0.0145 |

Contamination level per sample was predicted from allele counts as the location of the maximum of the posterior probability [symbol] (f|{n_{A},n_{R}}) (cf. Eq. 40).

TABLE 0.23
SNP Panel (Continued)

| Index | MAF | Depth Fraction |
|---|---|---|
| 36 | 0.3998 | 0.0146 |
| 37 | 0.3363 | 0.0150 |
| 38 | 0.2800 | 0.0151 |
| 39 | 0.3273 | 0.0152 |
| 40 | 0.4597 | 0.0159 |
| 41 | 0.4960 | 0.0160 |
| 42 | 0.4505 | 0.0161 |
| 43 | 0.4443 | 0.0162 |
| 44 | 0.4449 | 0.0164 |
| 45 | 0.4782 | 0.0164 |
| 46 | 0.3640 | 0.0179 |
| 47 | 0.4321 | 0.0183 |
| 48 | 0.4475 | 0.0192 |
| 49 | 0.2137 | 0.0192 |
| 50 | 0.3758 | 0.0196 |
| 51 | 0.4766 | 0.0202 |
| 52 | 0.3898 | 0.0222 |
| 53 | 0.2670 | 0.0223 |
| 54 | 0.4451 | 0.0236 |
| 55 | 0.3886 | 0.0236 |
| 56 | 0.2662 | 0.0252 |
| 57 | 0.4533 | 0.0272 |
| 58 | 0.3984 | 0.0275 |
| 59 | 0.4964 | 0.0299 |
| 60 | 0.3007 | 0.0315 |
| 61 | 0.4784 | 0.0348 |
| 62 | 0.3223 | 0.0353 |
| 63 | 0.2678 | 0.0412 |
| 64 | 0.4726 | 0.0422 |
| 65 | 0.3878 | 0.0423 |

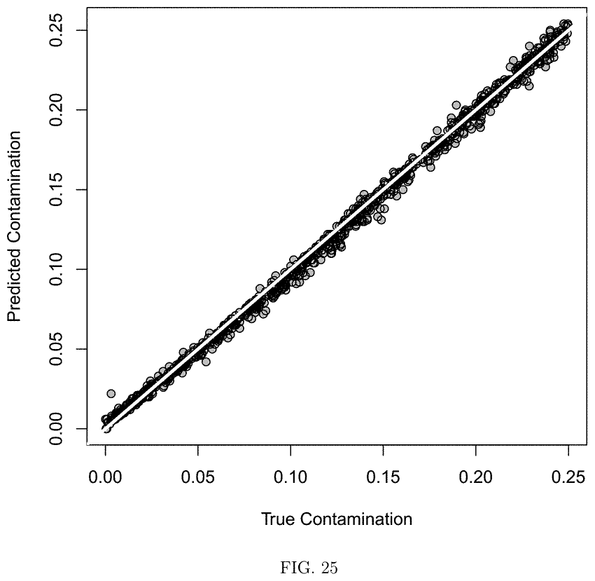

FIG. 25 shows the agreement between true and estimated contaminated levels in 1,000 simulated data sets. Mean, median, and standard deviation of the differences between true and predicted values are 0.15%, 0.12%, and 0.39%, respectively. In 96.7% of samples, both estimated and observed errors are lower than 1%.
S. Reduction to Practice of the System and Method to Measure Fetal Fraction from Minor Allele Counts

The human SNP panel tabulated in [symbol] . ?? was used to simulate 1,000 NIPT ccfDNA samples with fetal fraction uniformly sampled between 0 and 0.25. Total number of reads per sample was uniformly sampled between 30,000 and 80,000. Up to 4% of the 65 SNPs in the panel were randomly dropped out, with the fraction of failing loci uniformly varying from sample to sample between 0 and 4%. Actual read counts were generated using Poisson distribution with the mean given by the product of the locus-specific “Depth Fraction” value ( [symbol] . ??) and the total read count per sample. Reads were apportioned to reference and alternate alleles according to [symbol] . 0.3. Fetal fraction values f were estimated from simulated allele counts as the mean values of the posterior distributions [symbol] (f|{n_{R} n_{A}}) (cf. Eq. 54).

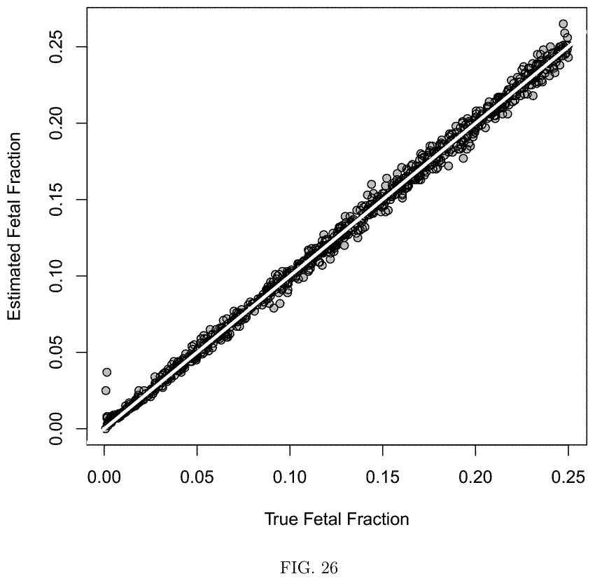

FIG. 26 illustrates the high degree of agreement between estimated and true fetal fraction values. Mean, median, and standard deviation of the differences between true and predicted values are 0.01%, 0.02%, and 0.42%, respectively.

T. Reduction to Practice of the System and Method to Detect Fetal Copy Number Variation from Minor Allele Counts

The human SNP panel tabulated in [symbol] . ?? was used to simulate 300 ccfDNA samples with fetal fraction values uniformly sampled between 0 and 0.25. The total numbers of reads per sample were uniformly sampled from 30,000 to 80,000. The fraction of failed loci per sample was uniformly sampled from 0 to 4%. In each sample, a different chromosome was randomly selected and the fetus was randomly assigned maternal deletion, paternal deletion, maternal duplication, or paternal duplication of the entire selected chromosome. All other fetal and maternal chromosomes are diploid. Total read counts per locus were generated using Poisson distribution with the mean given by the product of the locus-specific “Depth Fraction” value ( [symbol] . ??). Reference and alternate allele counts were simulated as Poisson random variables with mean values given by [symbol] . 0.7.

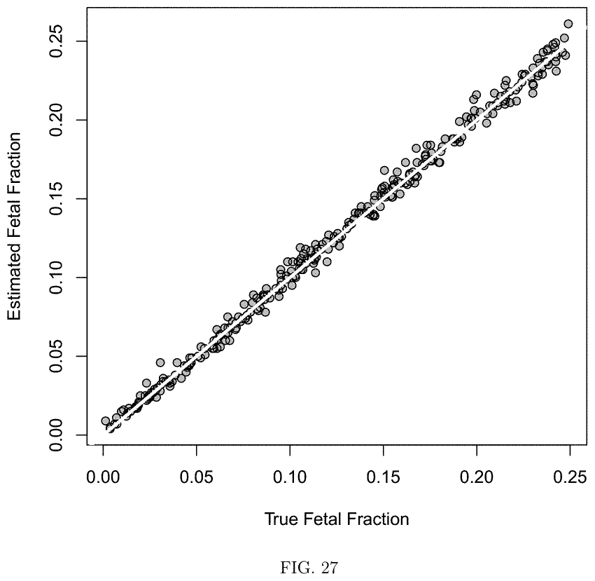

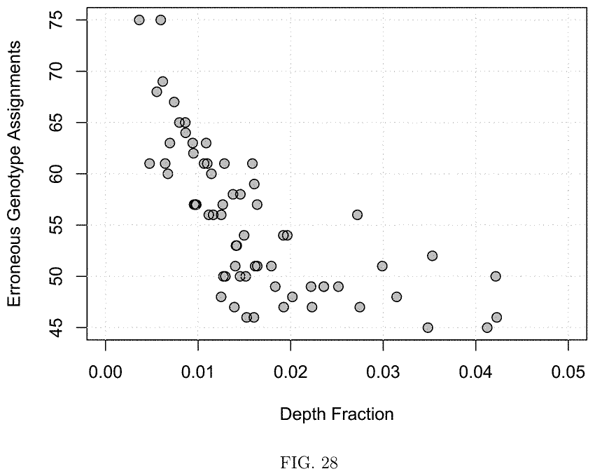

FIG. 27 shows the agreement between true fetal fractions and fetal fraction estimates obtained using the extended set of states, including non-diploid copy numbers. FIG. 28 shows that SNP loci that receive lower coverage depth have an increased genotype error rate.

U. Reduction to Practice of the System and Method to Haplotype Fetal Genome from scDNA or ccfDNA Minor Allele Counts and Phased Parental Genomes

The haplotyping scDNA algorithm was validated using two tests. First, a single homogeneous fetal region was generated containing 10,000 SNP loci. A set of phased parental alleles was simulated for each locus, with reference and alternate alleles treated as equally likely. A euploid parental transmission pattern was randomly chosen and assigned to the fetal region. Fetal scDNA SNP allele counts were generated using Poisson distribution as appropriate for the chosen parental transmission pattern (cf. Tbl. 0.14). Total number of reads per locus was uniformly sampled between 100 and 1,000. These simulated allele counts were then used to predict the phase and the parental transmission pattern. Next, the prediction was compared with the ground truth. Also, outliers (if any) were counted and recorded. This procedure was repeated 100 times. None of the 100 simulated data sets showed any discrepancies: predicted parental transmission patterns matched the ground truth in all informative loci. The uninformative loci (such as those where both parents are homozygous for the same allele) did not contradict predictions in any of the 100 simulated sets.

The second test generates a fetal genomic segment consisting of three homogeneous subregions. Each homogeneous subregion contains a variable number of SNPs. The actual number of SNP loci per homogeneous subregion is uniformly sampled between 40 and 100. Parental alleles are randomly assigned, again assuming equally probable reference and alternate alleles. Each homogeneous fetal subregion is randomly assigned a parental transmission pattern. Two neighboring subregions are prevented from having the same transmission pattern. Fetal allele counts are simulated using Poisson distribution with the total number of reads per locus randomly sampled between 100 and 1,000. Reference and alternate allele counts have expectation values as specified in [symbol] . 0.14 for the scDNA case. Finally, based on the simulated allele counts, fetal genome is segmented into homogeneous subregions and each subregion is assigned a predicted parental transmission pattern. Predictions are then compared with the ground truth and any discrepancies recorded. The procedure was repeated 100 times. Discrepancies were only observed in uninformative loci adjacent to recombination sites, sandwiched between two informative loci closest to the recombination site. Otherwise, all predicted phasing blocks and their parental transmission patterns matched the ground truth, and all predicted recombination sites were located within the uncertainty regions due to uninformative loci.

### W. Combinations of the Aspects of the Invention

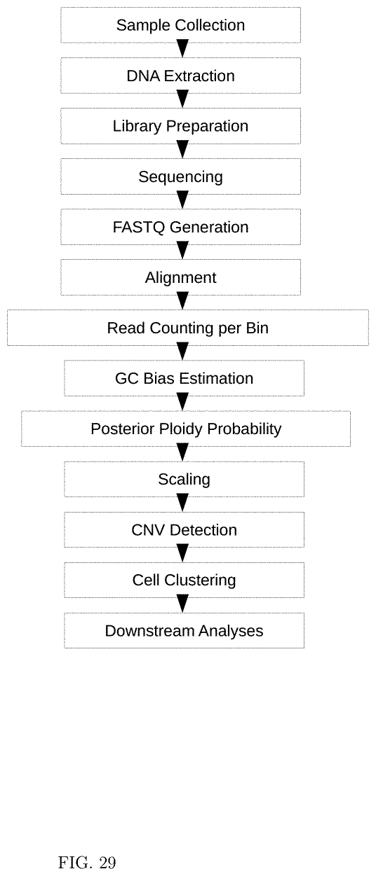

FIG. 29 shows an illustrative embodiment of a workflow combining some aspects of the invention related to single cell sequencing, copy number detection, and clustering.

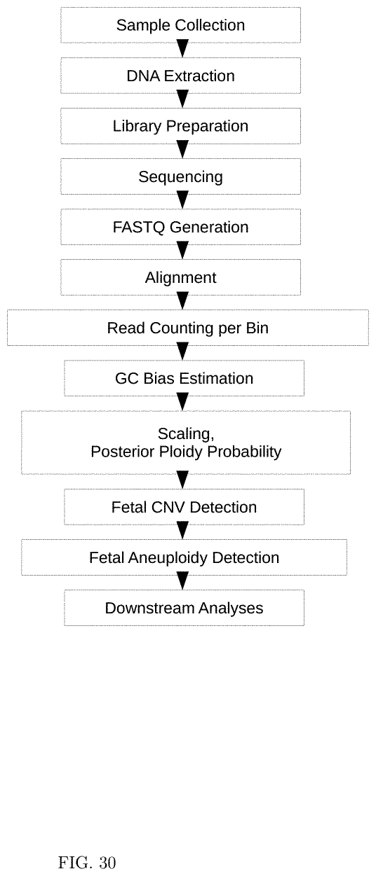

FIG. 30 shows an illustrative embodiment of a workflow combining some aspects of the invention related to sequencing of circulating cell-free DNA to detect fetal whole chromosome aneuploidies and/or fetal subchromosomal deletions or duplications.

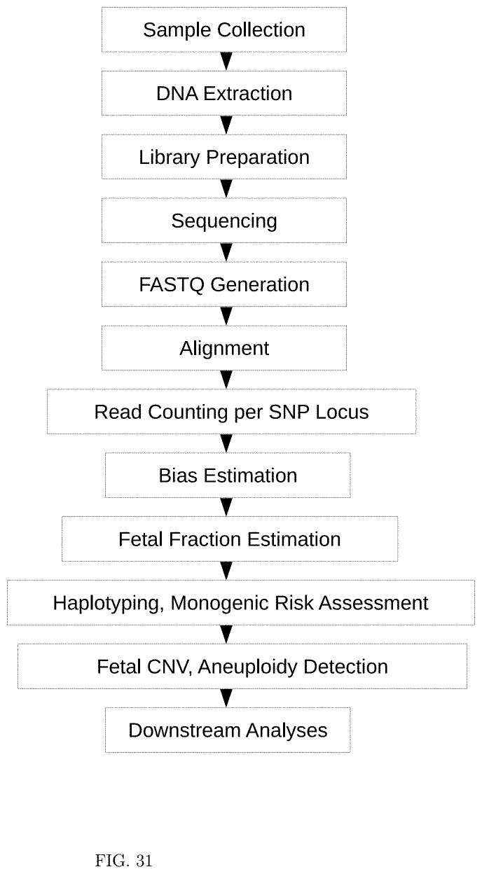

FIG. 31 shows an illustrative embodiment of a workflow combining some aspects of the invention related to fetal haplotyping from single cell DNA or circulating cell-free DNA, given known parental haplotype blocks and carrier status, with the goal of detecting parental transmission of monogenic diseases, as well as detection of fetal subchromosomal CNVs and/or whole chromosome aneuploidies.

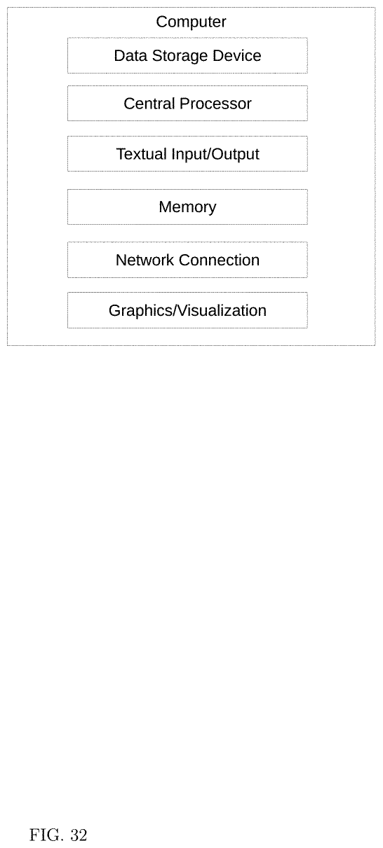

FIG. 32 shows an illustrative embodiment of a device in which certain embodiments of the invention may be implemented.

What is claimed:

1. System and method for analyses of nucleic acid sequencing data, comprising:
(a) acquisition of a biological specimen from a subject, (b) extraction of nucleic acids from the said specimen,
(c) physicochemical processing of the said extracted nucleic acids to create a sequencing library,
(d) sequencing of the said sequencing library to obtain sequencing data, and
(e) computational processing of the said sequencing data to detect presence or absence of genomic abnormalities in the said subject, wherein the said genomic abnormalities are reasonably expected to be relevant for the said subject's health, or for the health of an offspring of the said subject, or the health of a plurality of offsprings of the said subject, wherein the said offspring or the said plurality of offsprings may have already been born, or may have been conceived but not yet born, or may be conceived in the future.

2. The system and method of claim 1, wherein the said biological specimen is blood plasma containing circulating cell-free DNA (ccfDNA).

3. The system and method of claim 1, wherein the said biological specimen is a biological tissue comprising single cells or nuclei.

4. The system and method of claim 1, wherein the said biological specimen is urine containing circulating cell-free DNA.

5. The system and method of claim 2, characterized in that the said sequencing data from the said ccfDNA is computationally processed as a means to detect and, optionally, quantify presence or absence of contaminating DNA from an unrelated subject substantially as described with reference to Table 0.1 and Equations 36-43.

6. The system and method of claim 2, wherein:
(a) the said subject is a pregnant female, and
(b) the said ccfDNA comprises a mixture of maternal and fetal ccfDNA.

7. The system and method of claim 6, wherein the contribution of the said fetal ccfDNA to total said ccfDNA is quantified from measured SNP allele counts substantially as described with reference to Tables 0.2-0.3 and Equations 44-55.

8. The system and method of claim 2, wherein the amount of GC bias in the said sequencing data is quantified substantially as described with reference to Equations 1-12.

9. The system and method of claim 2, wherein the said sequencing data are obtained by whole-genome sequencing (WGS) procedure, and the said subject's ploidy across the subjects genome is quantified substantially as described with reference to Equations 13-14.

10. The system and method of claim 3, wherein the said sequencing data are obtained by whole-genome sequencing (WGS) procedure, and the resulting binned read count profile is scaled substantially as described with reference to Equations 16-21.

11. The system and method of claim 10, wherein the said scaled single cells' copy number profiles are normalized substantially as described with reference to Equations 13-14.

12. The system and method of claim 6, wherein the said sequencing data are obtained by whole-genome sequencing (WGS) procedure, and the fetal ploidy in the said maternal/fetal mixture across the fetal genome is quantified substantially as described with reference to Equations 22-23 as a means to detect fetal copy number variants.

13. The system and method of claim 3, wherein the copy-number variants in the said single cells or nuclei are detected substantially as described with reference to Equations 24-35.

14. The system and method of claim 11, wherein a plurality of said single cells copy number profiles are clustered into groups of similar single cell profiles substantially as described with reference to paragraphs 180-183.

15. The system and method of claim 3, wherein the maternally and paternally inherited haplotypes in the said subject's genome are determined from the measured SNP allele counts derived from the said single cell sequencing data and from the phased parental genomes substantially as described with reference to Tables 0.8-0.19 and Equations 56-65 as a means to non-invasively detect parentally transmitted monogenic diseases.

16. The system and method of claim 2, wherein the maternally and paternally inherited haplotypes in the neonatal subject's genome are determined from measured SNP allele counts derived from the said ccfDNA sequencing data substantially as described with reference to Tables 0.8-0.19 and Equations 56-65 as a means to non-invasively detect parentally transmitted monogenic diseases.

17. The system and method of claim 6, wherein the maternally and paternally inherited haplotypes in the fetal genome are determined from measured SNP allele counts derived from the said ccfDNA sequencing data substantially as described with reference to Tables 0.8-0.19 and Equations 56-65 as a means to non-invasively prenatally detect parentally transmitted monogenic diseases.

18. The system and method of claim 6, wherein the fetal CNVs are detected from measured SNP allele counts from said ccfDNA sequencing data substantially as described with reference to Tables to paragraphs 224-237 and Tables 0.4-0.7.

19. The system and method of claim 1 wherein the said subject is human.

20. The system and method of claim 1 wherein the said subject is non-human, comprising a domesticated animal (livestock, pet), a laboratory animal, or a wild animal.
